(19) United States
(12) Patent Application Publication
(10) Pub. No.: US 2025/0191680 A1
(43) Pub. Date: Jun. 12, 2025

(54) ANALYZING CELL PHENOTYPES

(71) Applicant: DEEPCELL, INC., Menlo Park, CA (US)

(72) Inventors: Stéphane C. BOUTET, Burlingame, CA (US); Andreja JOVIC, Sunnyvale, CA (US); Ryan C. CARELLI, Jersey City, NJ (US)

(73) Assignee: DEEPCELL, INC., Menlo Park, CA (US)

(21) Appl. No.: 18/649,826

(22) Filed: Apr. 29, 2024

Related U.S. Application Data
(60) Provisional application No. 63/608,106, filed on Dec. 8, 2023.

(51) Int. Cl. G16B 20/20; G06T 7/00

(52) U.S. Cl. CPC G16B 20/20; G06T 7/0012; G06T 2207/20036; G06T 2207/20081; G06T 2207/20084; G06T 2207/30024

(57) ABSTRACT

In some examples, a method of processing includes using a machine learning encoder to extract respective sets of machine learning (ML)-based features from respective images of viable, unstained cells. Cells of a first subset of the cells have a first genetic edit, and cells of a second subset of the cells lack the first genetic edit. The method may include using a computer vision encoder to extract respective sets of cell morphometric features from the respective images. The method may include using the respective sets of ML-based features and the respective sets of cell morphometric features to generate respective multi-dimensional feature vectors that represent respective cell phenotypes. The method may include using the respective multi-dimensional feature vectors to correlate, to the first genetic edit, a phenotypic difference between the cells of the first subset and the cells of the second subset.

### BACKGROUND

Clustered Regularly Interspaced Short Palindromic Repeats (CRISPR) and CRISPR-associated (Cas) proteins have revolutionized functional genomics by allowing for knockout (KO) or expression modulation of hundreds or thousands of genes in parallel. Links between gene KOs and corresponding cellular phenotypes have been characterized using phenotypic assays, single-cell RNA sequencing (scRNA-seq), and the like. However, these assays may be expensive and may not correlate strongly (or at all) with cell phenotype.

### SUMMARY

Provided herein is the use of deep learning and computer vision to analyze phenotypes of cells with genetic edits. Provided below are several examples that may be employed in any combination to achieve the benefits as described herein.

1. A method of processing, the method comprising:
using a machine learning encoder to extract respective sets of machine learning (ML)-based features from respective images of viable, unstained cells, wherein cells of a first subset of the cells have a first genetic edit, and wherein cells of a second subset of the cells lack the first genetic edit;
using a computer vision encoder to extract respective sets of cell morphometric features from the respective images;
using the respective sets of ML-based features and the respective sets of cell morphometric features to generate respective multi-dimensional feature vectors that represent respective cell phenotypes; and
using the respective multi-dimensional feature vectors to correlate, to the first genetic edit, a phenotypic difference between the cells of the first subset and the cells of the second subset.

2. The method of example 1, wherein the first genetic edit comprises a first gene knockout.

3. The method of example 2, wherein the first gene knockout is generated using Clustered Regularly Interspaced Short Palindromic Repeats (CRISPR) and a CRISPR-associated (Cas) protein.

5. The method of any one of examples 1 to 3, wherein the cells comprise a plurality of additional subsets having different genetic edits than one another.

6. The method of example 5, wherein the genetic edits are genome-wide.

7. The method of example 5, wherein the cells comprise at least 100 additional subsets having different genetic edits than one another.

8. The method of example 5, wherein the cells comprise at least 1,000 additional subsets having different genetic edits than one another.

9. The method of example 5, wherein the cells comprise at least 10,000 additional subsets having different genetic edits than one another.

10. The method of any one of examples 1 to 9, wherein the cells of the second subset of the cells have a second genetic edit that is different from the first genetic edit.

11. The method of any one of examples 1 to 10, wherein the cells of the second subset of the cells comprise a control.

12. The method of any one of examples 1 to 11, further comprising:
inputting the cells of the first subset of the cells to an inlet of a fluidic channel;
flowing the cells of the first subset of the cells from the inlet through the fluidic channel; and
generating the respective images of the cells of the first subset of the cells within the fluidic channel.

13. The method of example 12, wherein the cells of the first subset of cells are pooled with the second subset of cells, such that inputting the cells of the first subset of the cells to the inlet of the fluidic channel further comprises inputting the cells of the second subset of the cells to the inlet of the fluidic channel.

14. The method of example 12, wherein the cells of the second subset of the cells are input to the inlet of the fluidic channel separately from the cells of the first subset of the cells.

15. The method of any one of examples 12 to 14, further comprising collecting the cells of the first subset of the cells at an outlet of the fluidic channel.

16. The method of example 15, wherein the outlet comprises a first and second reservoirs, the method further comprising physically sorting the cells into the first reservoir or into the second reservoir using the phenotypic difference between the cells of the first subset and the cells of the second subset.

17. The method of example 16, further comprising performing a functional characterization of the cells sorted into the first reservoir.

18. The method of example 17, wherein the functional characterization is selected from the group consisting of: a cell migration assay or cell invasion assay.

19. The method of any one of examples 16 to 18, further comprising performing a molecular characterization of the cells sorted into the first reservoir.

20. The method of example 19, wherein the molecular characterization is selected from the group consisting of: single-cell RNA sequencing (scRNA-seq), single cell gene expression, single cell Assay for Transposase Accessible Chromatin (ATAC), combined single cell ATAC and gene expression, combined single cell gene expression and cell surface markers or intracellular proteins, single cell examination, bulk gene expression, bulk ATAC, bulk cell examination, or immunofluorescence.

21. The method of any one of examples 1 to 20, wherein the correlating comprises informatically linking the first genetic edit to a feature in the multi-dimensional feature vectors that is present in the cells of the first subset and is not present in the cells of the second subset.

22. The method of any one of examples 1 to 21, wherein the images are brightfield cell images.

23. The method of any one of examples 1 to 22, wherein the machine learning encoder uses a convolutional neural network or a vision transformer.

24. The method of any one of examples 1 to 23, wherein the computer-vision encoder uses a human-constructed algorithm.

25. The method of any one of examples 1 to 24, wherein the machine learning encoder extracts n ML-based features, the computer-vision encoder extracts m cell morphometric features, wherein the feature vectors have n+m dimensions, and wherein n and m are positive integers.

26. The method of example 25, wherein within each of the feature vectors, each dimension of the n+m dimensions is an element of that feature vector.

27. The method of example 26, wherein the element is a numeric value.

28. The method of any one of examples 1 to 27, wherein the ML-based features are orthogonal to one another.

29. The method of any one of examples 1 to 28, wherein the ML-based features are orthogonal to the cell morphometric features.

30. The method of any one of examples 1 to 29, wherein the cell morphometric features are selected from the group consisting of position features, cell shape features, pixel intensity features, texture features, and focus features.

31. The method of any one of examples 1 to 30, further comprising reducing dimensionalities of the multi-dimensional feature vectors to generate lower-dimensional vectors, wherein the lower-dimensional vectors are used to correlate, to the first genetic edit, the phenotypic difference between the cells of the first subset and the cells of the second subset.

32. The method of example 31, wherein the correlating comprises informatically linking the first genetic edit to a feature cluster in a space defined by the lower-dimensional vectors that is present in the cells of the first subset and is not present in the cells of the second subset.

Additional aspects and advantages of the present disclosure will become readily apparent to those skilled in this art from the following detailed description, wherein only illustrative examples of the present disclosure are shown and described. As will be realized, the present disclosure is capable of other and different examples, and its several details are capable of modifications in various obvious respects, all without departing from the disclosure. Accordingly, the drawings and description are to be regarded as illustrative in nature, and not as restrictive.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

Certain features of the examples described herein are set forth with particularity in the appended claims. A better understanding of the features and advantages of the present examples will be obtained by reference to the following detailed description that sets forth illustrative examples, in which the principles of the present disclosure are utilized, and the accompanying drawings (“FIG” herein), of which:

FIG. 1 schematically illustrates example operations and system components using deep learning and computer vision to analyze phenotypes of cells with genetic edits.

FIG. 2 schematically illustrates example operations and system components using deep learning and computer vision to analyze and sort phenotypes of cells with genetic edits.

FIG. 3 schematically illustrates an example method for classifying a cell.

FIG. 4 schematically illustrates, in one example, different ways of representing analysis data of image data of cells.

FIG. 5 schematically illustrates, in one example, different representations of analysis of image data of a population of cells.

FIG. 6 schematically illustrates, in one example, a method for a user to interact with a method for analyzing image data of cells.

FIG. 7 schematically illustrates, in one example, a cell analysis platform for analyzing image data of one or more cells.

FIGS. 8A-8B schematically illustrate, in one example, an example microfluidic system for sorting one or more cells.

FIG. 9 illustrates an example training architecture of the human foundation model.

FIG. 10 illustrates another example of the training architecture of the human foundation model shown in FIG. 9.

FIGS. 11A and 11B show examples of morphometric features of cellular images.

FIG. 12A illustrates cell classes, numbers of images used as training dataset to train the human foundation model, numbers of images processed by the human foundation model as test dataset, and corresponding representative cell images, in accordance with some examples of the present disclosure.

FIG. 12B illustrates an example confusion matrix between predicted cell classes classified by the human foundation model and actual cell classes, in accordance with some examples of the present disclosure.

FIG. 13 with views (a) to (f) schematically illustrate components of an example system for classifying and sorting one or more cells.

FIG. 14 with views (a) to (e) schematically illustrate operations that may be performed in an example method.

FIG. 15 shows an example computer system that is programmed or otherwise configured to implement methods provided herein.

FIG. 16 illustrates example reduced-dimensionality plots of features extracted from images of cells with different genetic edits than one another, and example images of cells having certain features.

FIG. 17 illustrates example reduced-dimensionality plots of features extracted from images of cells with different genetic edits than one another.

FIG. 18 illustrates example reduced-dimensionality plots of features extracted from images of cells with different genetic edits than one another, and example images of cells having certain features.

FIG. 19 illustrates example reduced-dimensionality plots of features extracted from images of cells with different genetic edits than one another.

FIG. 20 illustrates example reduced-dimensionality plots of features extracted from images of cells with different genetic edits than one another, and example images of cells having certain features.

FIG. 21 illustrates example reduced-dimensionality plots of features extracted from images of cells with different genetic edits than one another.

FIG. 22 illustrates example images of cells with different genetic edits than one another.

### INCORPORATION BY REFERENCE

All publications, patents, and patent applications mentioned in this specification are herein incorporated by reference in their entirety, to the same extent as if each individual publication, patent, or patent application is specifically and individually indicated to be incorporated by reference. To the extent publications and patents or patent applications incorporated by reference contradict the disclosure contained in the specification, the specification is intended to supersede and/or take precedence over any such contradictory material.

### DETAILED DESCRIPTION

Provided herein is the use of deep learning and computer vision to analyze phenotypes of cells with genetic edits.

More particularly, examples provided herein relate to orthogonal and high-resolution methods for characterizing cellular phenotypes and sorting cells which receive meaningful and phenotypically relevant perturbations. To this end, high-dimensional morphology profiling is performed on multiple genetically edited cell lines, e.g., CRISPR KO cell lines, to determine phenotypic differences arising from genetic perturbations, e.g., single gene perturbations. In some examples, the REM-I platform is used, which combines deep learning, computer vision, microfluidics, and high-resolution imaging to characterize and sort cells of potential interest. At least some of the cells (if not substantially all of the cells) may remain viable throughout the processing, and need not be stained in order to be imaged. Accordingly, the phenotypes of the cells may be accurately analyzed. Moreover, the sorted cells readily may be subjected to further analysis, such as functional characterization or molecular characterization, e.g., in a manner such as described herein. In comparison, previously known imaging methods may require cell staining and fixation, which may adversely perturb cells' phenotype and inhibit further functional or molecular characterization of the cells.

First, some example terms will be defined. Then, some example uses of deep learning and computer vision to analyze phenotypes of cells with genetic edits will be described. Then, further details of selected operations and system components, such as for deep learning and computer vision, microfluidics, and imaging, will be described.

### Terms

Unless defined otherwise, all technical and scientific terms used herein have the same meaning as commonly understood by one of ordinary skill in the art to which the present disclosure belongs. In case of conflict, the present application including the definitions will control. Also, unless otherwise required by context, singular terms shall include pluralities and plural terms shall include the singular.

Relative terms, such as “about,” “substantially,” or “approximately” are used to include small variations with specific numerical values (e.g., +/−x %), as well as including the situation of no variation (+/−0%). In some examples, the numerical value x is less than or equal to 10—e.g., less than or equal to 5, to 2, to 1, or smaller.

As used herein, when referring to a cell, the term “phenotype” is intended to refer to the observable properties of the cell. A cell's phenotype includes, among other things, the cell's genome, epigenome, transcriptome, proteome, and morphology, as well as the mechanisms of regulation affecting such properties.

As used herein, the terms “morphology” and “morphological feature” are intended to refer to the form, structure, or configuration of the cell. The morphology of a cell may include one or more aspects of a cell's appearance, such as shape, size, arrangement, form, structure, pattern(s) of one or more internal and/or external parts of the cell, or shade (e.g., color, greyscale, texture, etc.). Non-limiting examples of a shape of a cell may include, but are not limited to, circular, elliptic, shmoo-like, dumbbell, star-like, flat, scale-like, columnar, invaginated, having one or more concavely formed walls, having one or more convexly formed walls, prolongated, having appendices, having cilia, having angle(s), having corner(s), etc. Certain morphological features of a cell need not be stained in order to be visible in an image of the cell. Certain morphological feature(s) of a cell which would not have otherwise been visible in an image of the cell may be rendered visible, in that images, using a stain (e.g., small molecule or antibody staining).

The term “morphometric feature” is intended to refer to a quantitative representation of a morphological feature of a cell. In some cases, the terms “morphological feature” and “morphometric feature” are used interchangeably herein, for example where an image is being processed that includes a morphological feature.

As used herein, when referring to a cell, the terms “unstained” and “tag-free” refers to a cell that has not been treated in such a manner as render visible any morphological feature, in an image of the cell, that would not have otherwise been visible in that image without the treatment.

As used herein, the term “viable cell” refers to a cell that is not undergoing necrosis or a cell that is not in an early or late apoptotic state. Assays for determining cell viability may include, e.g., as using propidium iodide (PI) staining which may be detected by flow cytometry. A “viable cell” as disclosed herein may be characterized by exhibiting one or more characteristics (e.g., morphology, one or more gene expression profiles, etc.) that is substantially unaltered (or that is not substantially impacted by) by certain operations or processes disclosed herein (e.g., partitioning, imaging, or sorting). In some examples, a characteristic of a viable cell may be a gene transcript accumulation rate, which may be characterized by a change in transcript levels of a same gene (e.g., a same endogenous gene) between mother and daughter cells over the time between cell divisions, as ascertained by single cell sequencing, polymerase chain reaction (PCR), etc. However, it will be understood that a genetic edit may affect the morphology, gene expression profile(s), or gene transcript accumulation rate of a cell, and the cell nonetheless may remain viable. Some genetic edits may render the cell non-viable.

As used herein, a “genetic edit” or “perturbation” refers to a change to the DNA of an organism relative to the native DNA of the organism. In some examples, genetic edits may be permanent, while in other examples, genetic edits may be transient. In some examples, a genetic edit may be or include a knockout (e.g., CRISPR, siRNA, or shRNA, including transient or permanent modifications), a knockin, or an overexpression of a specific gene. A genetic edit may include adding one or more nucleotides to the organism's DNA, removing one or more nucleotides from the organism's DNA, or substituting one or more nucleotides in the organism's DNA with one or more different nucleotides. In nonlimiting examples in which one or more nucleotides are removed from the organism's DNA, the genetic edit may be referred to as a “knockout” or “KO.” A variety of tools may be used to make a genetic edit, such as Clustered Regularly Interspaced Short Palindromic Repeats (CRISPR), which may be used with a CRISPR-associated (Cas) protein such as Cas9; or an engineered zinc finger nuclease. A cell the genome of which is edited using CRISPR may be referred to as having a “CRISPR perturbation.” In nonlimiting examples in which one or more nucleotides are removed from the organism's DNA using CRISPR, the genetic edit may be referred to as a “CRISPR KO.”

As used herein, the term “real-time” generally refers to operations that are performed relatively close in time to one another. Non-limiting examples of real-time pairs of operations may include: flowing a cell through an imaging area of a fluidic channel and generating an image of that cell; generating an image of a cell and extracting features from that image; extracting features from an image and generating a multi-dimensional feature vector; generating a multi-dimensional feature vector and reducing dimensionality of the multi-dimensional feature vector to generate a lower-dimensional vector; generating a lower-dimensional vector and using the lower-dimensional vector to analyze a cellular phenotype; or analyzing a cellular phenotype and sorting the cell into a reservoir using the cellular phenotype. In some examples, real-time operations may be performed almost immediately or within a short enough time span, such as within at most about 1 second, for example at most about 0.1 seconds, at most about 0.01 seconds, at most about 1 ms, at most about 0.1 ms, at most about 0.01 ms, at most about 0.001 ms, at most about 0.0001 ms, or less, relative to one another. In some examples, any of the operations of a computer processor as provided herein may be performed (e.g., automatically performed) in real-time.

As used herein, an “encoder” refers to a type of model that transforms or “encodes” an image into a vector. Nonlimiting examples of encoders include machine learning-based models, and computer vision models.

Using Deep Learning and Computer Vision to Analyze Phenotypes of Cells with Genetic Edits

Morphology is an important cell property associated with identity, state, and function, but in some instances it is characterized crudely in a few standard dimensions such as diameter, perimeter, or area, or with subjective qualitative descriptions. Cell morphology information has historically been used for cell and disease characterization but has been difficult to objectively and reproducibly quantify. Cell morphology in many instances is studied qualitatively through a microscope, which may be inherently slow, difficult to scale, and relies on human interpretation.

Additionally, cells are sometimes no longer amenable to additional downstream studies, such as flow cytometry or single cell sequencing, after being subjected to antibody staining or destructive analytical processes such as cell lysis. Current sorting methods such as fluorescence-activated cell sorting (FACS) rely on a limited set of biomarkers, which cannot cover the full extent or be readily available for all distinct cell properties. Additionally, dependence on antibodies, dyes/stains, and biomarkers to denote cell identity may inadvertently create sampling bias by depleting biomarker-negative but potentially biologically interesting cell populations.

In comparison, the present disclosure provides multi-dimensional morphology analysis (e.g., profiling) enabled by machine learning and computer vision morphometrics. The present disclosure has the benefit of enabling higher resolution and biological insight while reducing labor-intensive cell processing manipulations. The multi-dimensional morphology profiling and sorting of unlabeled single cells using machine learning, advanced imaging, and microfluidics may be used to assess population heterogeneity beyond biomarkers. In particular, a deep learning model may be used provide quantitative descriptions of cell features using one or more neural network, and a computer vision model may provide a quantitative assessment of cell and biological features using discrete image analysis algorithms. Some examples of the present disclosure provide a method of processing that includes using a machine learning encoder to extract a set of ML-based features from a cell images, using a computer vision encoder to extract a set of cell morphometric features from the cell image, and using the set of ML-based features and the set of cell morphometric features to generate a feature vector that represents morphology of the cell. The feature vector may be used to analyze phenotypes of cells with genetic edits. The method as described herein may allow for extracting and interpreting cell morphology features with a multidimensional, unbounded, and quantitative assessment. At least some of the genetically edited cells may be viable, and may be unstained (label-free), thus facilitating further functional or molecular analysis of the cells.

In some examples, the present disclosure provides a system for cell morphology analysis. In some examples, the system may comprise a benchtop single-cell imaging and sorting system for high-dimensional morphology analysis. The system may combine label-free imaging, deep learning, computer vision morphometrics, and gentle cell sorting to leverage multidimensional single cell morphology as a quantitative readout. The system may capture high-resolution brightfield cell images, from which features (e.g., dimensional embedding vectors) may be extracted representing the morphology of the genetically edited cells.

The system and method may provide a relatively fast workflow for cell morphology analysis. For example, it may only take a few hours from preparing cell samples to generating publishable figures representing cell morphology. In some examples, the systems and methods as described herein may be used in Clustered Regularly Interspaced Short Palindromic Repeats (CRISPR) perturbation screening, using cell morphology as a novel biomarker for the screening. The systems and methods as described herein may be used in sample-level profiling, including but not limited to heterogeneous sample evaluation and characterization, phenotype (e.g., disease) detection and enrichment, and sample clean-up. In other examples, the systems and methods as described herein may be used in cell-level phenotyping, including cell health status, cell state characterization, and multi-omic integration.

The systems and methods disclosed herein have a variety of potential uses. For example, the combination of deep learning and computer vision morphometrics may allow cell characterization and sorting based on multi-dimensional morphometric and deep learning derived features, which may be used to identify and enrich genetically edited cells in heterogeneous populations (e.g., populations of cells at least some of which cells have different genetic edits than one another). Quantitative multi-dimensional morphology information at single cell level may provide additional information to assess the respective effects of different genetic edits. Cell populations may be characterized with specific morphological profiles have distinct functional and molecular profiles, and morphologically distinct cells (e.g., control versus genetically edited, or one genetic edit versus a different genetic edit) may be distinguished over each other.

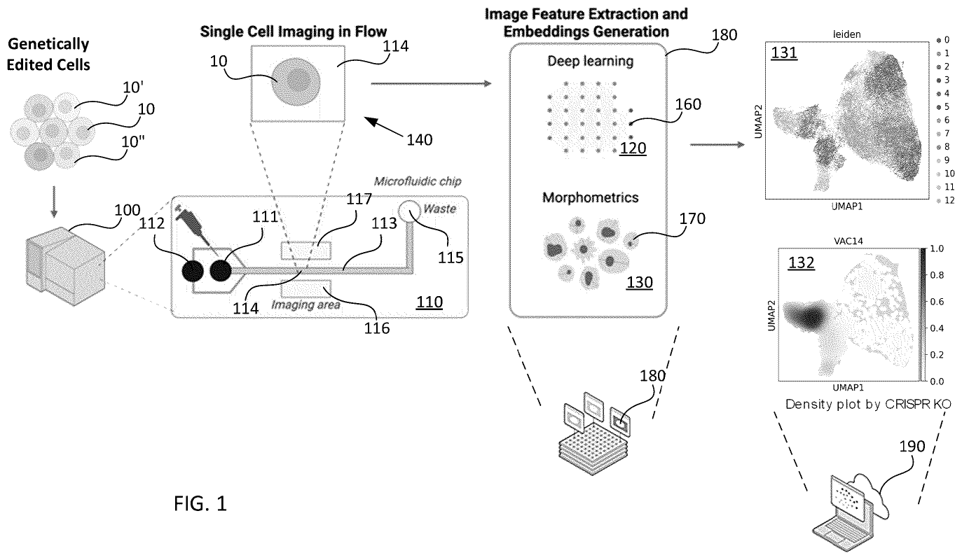

FIG. 1 schematically illustrates example operations and system components using deep learning and computer vision to analyze phenotypes of cells with genetic edits. In the nonlimiting example illustrated in FIG. 1, genetically edited cells may be input into system 100. In this example, the genetically edited cells include a first subset of cells 10, a second subset of cells 10′, and a third subset of cells 10″. Cells 10 of the first subset of cells may have a first genetic edit, for example a first CRISPR perturbation. In some examples, the first genetic edit is or includes a first gene KO. In nonlimiting examples in which the first gene KO is generated using CRISPR and a Cas protein (such as Cas9), the first gene KO may be referred to as a first CRISPR KO. Cells 10′ of a second subset of cells may lack the first genetic edit. Cells 10′ may have a second genetic edit that is different from the first genetic edit, for example a second CRISPR perturbation that is different from the first CRISPR perturbation. In some examples, the second genetic edit is or includes a second gene KO that is different from the KO of the cells 10. In nonlimiting examples in which the second gene KO is generated using CRISPR and a Cas protein (such as Cas9), the second gene KO may be referred to as a second CRISPR KO. In some examples, cells 10″ may include a third genetic edit that is different from those of cells 10 and cells 10′. In other examples, cells 10″ may be control cells, which may lack any genetic edit. In some examples, cells 10″ may otherwise be treated similarly as cells 10 and cells 10′, e.g., may be exposed to Cas protein but without guide RNA that would otherwise enable the Cas to genetically edit cells 10. At least some of the cells 10, 10′. 10″ may be viable, although it will be appreciated that certain genetic edits may render the respective cells non-viable.

Although three subsets of cells are illustrated in FIG. 1, it will be appreciated that any suitable number of subsets of cells may be input into system 100. In one nonlimiting example, cells having genome-wide edits may be input into system 100. The cells may include at least 100 additional subsets having different edits than one another, or may include at least 1,000 additional subsets having different edits than one another, or may include at least 10,000 additional subsets having different edits than one another.

The cells 10, 10′, and 10″ may be flowed through a microfluidic platform, and respective images of the cells may be generated within the microfluidic platform. For example, as illustrated in FIG. 1, system 100 may include a microfluidic chip 110 that may be used to obtain single cell images of cells in flow. Microfluidic chip 110 may include first inlet 111, second inlet 112, fluidic channel 113 including imaging area 114, and output 115 (e.g., waste receptacle). Cells 10 of the first subset of cells 10 may be input into first inlet 111, may be flowed from first inlet 111 and through fluidic channel 113. Second inlet 112 may be used to introduce a flow of a fluid (such as a buffer) that is used to focus the cells within fluidic channel 113. Respective images of cells 10 of the first subset of cells 10 may be generated within fluidic channel 113. For example, system 100 may include any suitable number of imaging devices for generating respective images 140 of single cells as they flow through imaging area 114 of fluidic channel 113. Boxes 116, 117 illustrated in FIG. 1 are markers on the chips to enable automated chip alignment; in some examples, these boxes include a series of “micro posts” that have different shapes based on location that guides the algorithm to the same imaging area of the chip in 113. Further details of imaging devices, and of microfluidic chips (cartridges), are described elsewhere herein.

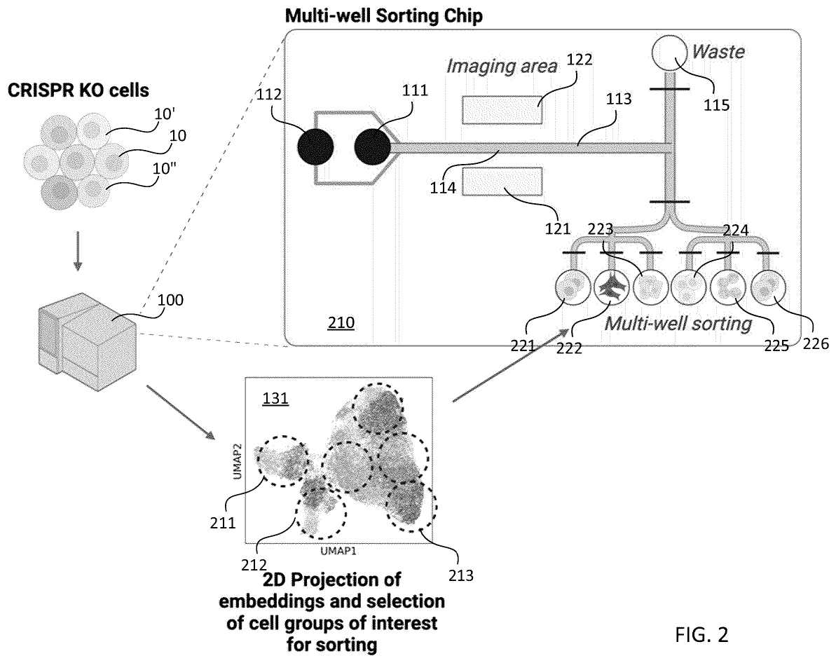

In some examples, different subsets of cells (which may have different genetic edits than one another) may be pooled with one another prior to imaging. For example, as illustrated in FIG. 1, the cells 10 of the first subset of cells 10 are pooled with the second subset of cells 10′ (and optionally also the third subset of cells 10″). As such, inputting the cells 10 of the first subset of the cells 10 to the inlet 111 of the fluidic channel 113 further may include inputting the cells 10′ of the second subset of the cells” (and optionally also the third subset of cells 10″) to the inlet of the fluidic channel. Alternatively, different subsets of cells (which may have different genetic edits than one another) may be processed, and imaged, separately. For example, the cells 10′ of the second subset of the cells 10′ may be input to the inlet of the fluidic channel separately from the cells 10 of the first subset of the cells 10 (as well as separately from the third subset of cells 10″). Regardless of whether the different subsets of cells are pooled or not pooled, the cells may be collected after the images of the cells are generated. In the example shown in FIG. 1 the cells may be collected at outlet 115 of the fluidic channel 113. In other examples such as will be described with reference to FIG. 2, the cells may be sorted into a plurality of collection wells using morphologies of the cells. For example, the outlet may include multiple reservoirs, and the cells may be sorted into respective reservoirs using phenotypic differences between the cells.

System 100 also may include a machine learning encoder to extract respective sets of machine learning (ML)-based features from respective images of cells having genetic edits, such as CRISPR perturbations; and a computer vision encoder to extract respective sets of cell morphometric features from the respective images. System 100 also may be configured to use the respective sets of ML-based features and the respective sets of cell morphometric features to generate respective multi-dimensional feature vectors that represent morphologies of the cells. System 100 also may be configured to use the respective feature vectors to screen the CRISPR perturbations.

For example, system 100 may use a human foundation model (HFM) for cell morphology analysis (e.g., profiling). The human foundation model may combine a deep learning model and a computer vision model and extract cell features from cell images. In some examples, the deep learning model may process cell images as input and provide quantitative descriptions of cell features. The deep learning model may extract deep learning features that are information-rich metrics of cell morphology with powerful discriminative capabilities. The deep learning features may not be human-interpretable. The computer vision model may process cell images as input and provide morphometric features that are human-interpretable, quantitative metrics of cell morphology including cell size, shape, texture, and intensity. The morphometrics may be computationally generated using discrete computer vision algorithms. When some of the morphometrics are too computationally intensive to compute in real time, the deep learning model may overcome the limitation of the computer vision model by imputing the most computationally intensive morphometrics into the human foundation model. By combining deep learning and morphometric features, the human foundation model as described herein may provide both accuracy and interpretability in real-time feature extraction, cell classification and sorting. The human foundation model may also have strong generalization capabilities that enable hypothesis-free sample exploration and efficient generation of application-specific models.

As illustrated in FIG. 1, the human foundation model may be used to extract features associated with cell morphology from cell images, in accordance with some examples of the present disclosure. The human foundation model may process cell images 140 and generate features therefrom. In some examples, cells that are under analysis may be unstained, and the cell images 140 may be brightfield cell images.

In some examples, the human foundation model may comprise a deep learning model 120 and a computer vision model 130. The deep learning model 120 may comprise a deep learning encoder, for example, a convolutional neural network. The deep learning model 120 may process cell images 140 as input and extract artificial intelligence (AI) features therefrom. In some examples, the AI features may comprise deep learning features 160, e.g., features that are extracted using a deep learning algorithm, such as a convolutional neural network or vision transformer, with other nonlimiting examples being provided elsewhere herein. In some examples, the dimensions of the deep learning features may be in a range of between about 1 and about 10, between about 1 and about 100, between about 1 and about 1,000, between about 1 and about 10,000, or between about 1 and about 100,000, or any value between any of the aforementioned numbers. Other suitable numbers are also possible. As one example, the deep learning model 120 may extract between about 5 and about 1000 deep learning features 160, e.g., between about 10 and about 500 deep learning features, e.g., between about 50 and about 100 deep learning features, from each cell image. In some examples, in a data set comprising a plurality of deep learning features of the cell(s), each feature may be referred to as a dimension (e.g., a deep learning dimension). Any range of dimensions of the deep learning features may be contemplated, for example from 1 through any number greater than about 100,000. As illustrated in FIG. 1, as one nonlimiting example, the deep learning model 120 generates about 64-dimensional deep learning features 160.

In some examples, the computer vision model 130 may comprise a computer vision encoder including human-constructed algorithm(s), which in some cases may be referred to as “rule-based morphometrics.” The computer vision model 130 may process cell images 140 as input and extract cell features therefrom. In some examples, the cell features may comprise cell position features, cell shape features, pixel intensity features, texture features, focus features, or combinations thereof. The cell features may comprise morphometric features 170. Nonlimiting examples of morphometric features 170 are provided below in Table 2. The dimensions of the morphometric features 170 may be in a range of between about 1 and about 10, between about 1 and about 100, between about 1 and about 1,000, between about 1 and about 10,000, or between about 1 and about 100,000, or any value between any of the aforementioned numbers. The cell features may include any suitable number of morphometric features 170, for example, at least about 1 feature, e.g., at least about 10 features, at least about 100 features, at least about 1,000 features, at least about 10,000 features, or at least about 100,000 features or more. In some cases, in a data set comprising a plurality of computer vision features of the cell(s), each feature may be referred to as a dimension (e.g., computer vision-based dimension). Any range of dimensions of the morphometric features may be contemplated, for example from 1 through any number greater than 100,000. As one example, the computer vision model 130 may extract between about 5 and about 1000 morphometric features, e.g., between about 10 and about 500 morphometric features, e.g., between about 50 and about 100 features, and any values in between any of the aforementioned ranges, from each cell image. As illustrated in FIG. 1, in one nonlimiting example, the computer vision model generates about 51-dimensional morphometric features 170.

In some examples, the human foundation model may encode the deep learning features 160 and morphometric features 170 into multi-dimensional numerical vectors representing the cell morphology. For example, the machine learning encoder 120 may extract n ML-based features 160, the computer-vision encoder 130 may extract m cell morphological features 170, and the multi-dimensional feature vectors may have n+m dimensions. In one nonlimiting example, 64-dimensional deep learning features 160 and 51-dimensional morphometric features 170 may be encoded into 115-dimensional embedding vectors representing the cell morphology. Within each of the multi-dimensional feature vectors, each dimension of the n+m dimensions may be an element of that multi-dimensional feature vector. The element may be a numeric value. In some examples, the ML-based features 160 may be orthogonal to one another. In some examples, the ML-based features 160 may be orthogonal to the cell morphological features 170. Further details regarding example data structures (e.g., multi-dimensional feature vectors) for encoding ML-based features 160 and morphometric features 170 are provided further below.

In some examples, the human foundation model 180 may generate one or more morphology maps based on deep learning features 160, morphometric features 170, or combinations thereof and in some examples based on a plurality of deep learning features and a plurality of morphometric features (e.g., based on a multi-dimensional feature vector that represents morphology of a cell). The generation of a morphology map may be referred to as reducing dimensionalities of multi-dimensional feature vectors to generate lower-dimensional vectors. A cell morphology map may be a visual (e.g., graphical) representation of one or more clusters of datapoints. The cell morphology map may be a 1-dimensional (1D) representation (e.g., based on one morphometric feature as one parameter or dimension) or a multi-dimensional representation, such as a 2-dimensional (2D) representation (e.g., based on two morphometric features as two parameters or dimensions), a 3-dimensional (3D) representation (e.g., based on three morphometric features as three parameters or dimensions), a 4-dimensional (4D) representation, etc. In some examples, one morphometric feature of a plurality of morphometric features used for blotting the cell morphology map may be represented as a non-axial parameter (e.g., non-x, y, or z axis), such as, distinguishable colors (e.g., heatmap), numbers, letters (e.g., texts of one or more languages), and/or symbols (e.g., a square, oval, triangle, square, etc.). For example, a heatmap may be used as colorimetric scale to represent the classifier prediction percentages for each cell against a cell class, cell type, or cell state.

The cell morphology map may be generated based on one or more morphological features (e.g., characteristics, profiles, fingerprints, etc.) from the processed image data. Non-limiting examples of one or more morphometric features of a cell, as disclosed herein, that may be extracted from one or more images of the cell may include, but are not limited to (a) shape, curvature, size (e.g., diameter, length, width, circumference), area, volume, texture, thickness, roundness, etc. of the cell or one or more components of the cell (e.g., cell membrane, nucleus, mitochondria, etc.), (ii) number or positioning of one or more contents (e.g., nucleus, mitochondria, etc.) of the cell within the cell (e.g., center, off-centered, etc.), and (iii) optical characteristics of a region of the image(s) (e.g., unique groups of pixels within the image(s)) that correspond to the cell or a portion thereof (e.g., light emission, transmission, reflectance, absorbance, fluorescence, luminescence, etc.).

One or more dimensions of the cell morphology map may be represented by various approaches (e.g., dimensionality reduction approaches), such as, for example, principal component analysis (PCA), multidimensional scaling (MDS), t-distributed stochastic neighbor embedding (t-SNE), and uniform manifold approximation and projection (UMAP). For example, UMAP may be a machine learning technique for dimension reduction. UMAP may be constructed from a theoretical framework based in Riemannian geometry and algebraic topology. UMAP may be utilized for a practical scalable algorithm that applies to real world data, such as morphometric features of one or more cells.

The respective multi-dimensional feature vectors may be used to correlate, to the first genetic edit, a phenotypic difference between the cells of the first subset and the cells of the second subset, e.g., to screen the CRISPR perturbations. For example, to identify whether a group of cells that have been genetically modified is different from a control group of cells, the distribution of cells across the different Leiden clusters may be compared. Such comparison may be used to show that when a cell is genetically modified and experiences a modification of morphology (whether an obvious phenotypic change or a change that is not apparent to the human eye), cell distribution will change in one or more Leiden clusters. This difference also may be seen by visualizing the changes of density of cell distribution in density plots in map space. In some examples, a classifier (such as a random forest classifier) may be used to demonstrate and quantify how cells with different morphotypes are different. In some examples, a defined cell and all of its genetic edits (e.g., CRISPR modifications, such as CRISPR KOs) removing protein expression, may be considered to define for this cell all the morphological space that this cell may “occupy”. In some examples, this morphological space specific to the cell may be considered to essentially define the cell morphology allowed by the expressed genome. In some examples, the correlating includes informatically linking the first genetic edit to a feature in the multi-dimensional feature vectors that is present in the cells of the first subset and is not present in the cells of the second subset. In some examples, the correlations are performed in a reduced dimensionality space. For example, the correlating may include informatically linking the first genetic edit to a feature cluster in a space defined by the lower-dimensional vectors that is present in the cells of the first subset and is not present in the cells of the second subset. In some examples, the lower-dimensional vectors obtained by reducing dimensionalities of the multi-dimensional feature vectors may be used to screen the CRISPR perturbations.

For example, as illustrated in FIG. 1, system 100 may include data suite 190 that uses the multi-dimensional feature vectors to generate and display a two-dimensional projection of embeddings 131, e.g., UMAP plot as described in greater detail below. The respective dimensions of projection 131 may be first and second UMAP dimensions that data suite 190 generates by reducing the dimensionality of the multi-dimensional feature vectors that the human foundation model 180 generates. In the example illustrated in FIG. 1, the two-dimensional projection of embeddings 131 includes two-dimensional projections of embeddings for a plurality of cells with different genetic edits than one another, in which the cells designated “0” are control cells, and wherein cells designated 1, 2, . . . 12 have different genetic edits than one another. Table 5, described further below in Example 2, lists the cell lines, and genetic edits thereof, corresponding to the designations 0, 1, 2, . . . 12 in FIG. 1. It will be appreciated that the operations, systems, and platforms herein readily may be applied to processing images of other cell lines, and other genetic edits. Data suite 190 may use clustering, e.g., Leiden clustering or other suitable clustering algorithm such as described herein, to generate the two-dimensional projection of embeddings 131 in such a manner that the projection includes the statistically most relevant features, e.g., the features that the data suite 190 determines to best distinguish cells from one another in the two-dimensional projection of embeddings 131—that is, to identify phenotypic differences between different subsets of cells. Such clustering may include using the multi-dimensional feature vectors to train a classifier in a manner such as described in greater detail below. In some examples, before using Leiden clustering, data suite 190 normalizes the embeddings, and removes data showing excessive standard deviation. In some examples, the data suite 190 then may reduce the data dimensionality, for example, using principle component analysis (PCA), which reveals the main variation(s) in the data. In some examples, the data suite then may calculate the neighborhood graphs of cells using the PCA representation, and then embed the graph in two dimensions using a UMAP algorithm. In some examples, the neighborhood graphs then may be clustered using the Leiden-clustering method (community based on optimizing modularity).

In the example illustrated in FIG. 1, data suite 190 also generates a two-dimensional density plot of embeddings 132 for a single cell type of cells (e.g., HEK293 cells with the VAC14 gene knocked out, in the nonlimiting example shown in FIG. 1 and as described in greater detail below with reference to Example 2). In some examples, the density plot 132 illustrates the distribution of cells with a particular morphotype, and illustrate in a simple manner how that particular morphotype is represented at the cell population level. While the Leiden clustering of embeddings 131 illustrate the number of cells in each cluster and how the cells change from one condition to the other, the embeddings 131 may omit the cell population distribution representation in order to reduce or avoid overcrowding of that representation. Accordingly, it may be understood that embeddings 131 and density plot 132 are complementary to one another.

In some examples, data suite 190 may generate an interface via which a user may select between (i) the two-dimensional projection of embeddings 131 for the plurality of subsets of cells with different genetic edits than one another, and (ii) the two-dimensional projection of embeddings 132 for a selected subset cells with a particular genetic edit.

In some examples, after the respective images of the cells are generated, the cells are collected. In the nonlimiting example illustrated in FIG. 1, the multi-dimensional feature vectors may be used to train a classifier to recognize the statistically most relevant features for distinguishing, from one another, cells with different genetic edits than one another, but the cells may collected in the same collection well as one another, or simply discarded as waste. FIG. 2 schematically illustrates example operations and system components using deep learning and computer vision to analyze and sort phenotypes of cells with genetic edits. In the nonlimiting example illustrated in FIG. 2, the cells are sorted into a plurality of collection wells using morphologies of the cells. For example, system 100 may use or include multi-well sorting chip 210 which may be configured, in some regards, similarly as microfluidic chip 110. For example, multi-well sorting chip 210 may include first inlet 111, second inlet 112, fluidic channel 113 including imaging area 114, and first output 115 (e.g., waste receptacle). Cells 10 of the first subset of cells may be input into first inlet 111, may be flowed from first inlet 111 and through fluidic channel 113. Respective images of cells 10 of the first subset of cells may be generated within fluidic channel 113. For example, system 100 may include any suitable number of imaging devices for generating respective images 140 of single cells as they flow through imaging area 114 of fluidic channel 113. Multi-well sorting chip 210 further may include at least first and second reservoirs for collecting sorting cells, and cells may be sorted into the first reservoir or into the second reservoir using the phenotypic difference between the cells 10 of the first subset and the cells 10′ of the second subset. In the nonlimiting example shown in FIG. 2, multi-well sorting chip 210 includes reservoirs 221, 222, 223, 224, 225, and 226 into which cells are sorted using phenotypic differences between the cells. It will be appreciated that the chip may include any suitable number of reservoirs. Nonlimiting examples of methods and mechanisms for sorting cells are described further below.

In some examples, cell-level phenotyping may be performed on any suitable one(s) of the sorted cells. Such phenotyping may include a molecular characterization of the cells sorted into a given reservoir, a functional characterization (functional screening) of the cells sorted into that reservoir, or both a molecular characterization and a functional characterization of the cells sorted into that reservoir. Non-limiting examples of cell-level phenotyping that includes molecular characterization may include a multi-omic assay, illustratively single-cell RNA sequencing (scRNA-seq), single cell gene expression, single cell Assay for Transposase Accessible Chromatin (ATAC), combined single cell ATAC and gene expression, combined single cell gene expression and cell surface markers or intracellular proteins (e.g., phosphorpho-proteins or other signaling proteins), single cell examination (e.g., targeted genomic sequencing or whole genomic sequencing of a single cell), bulk gene expression, bulk ATAC, bulk cell examination (e.g., targeted genomic sequencing or whole genomic sequencing of a group of cells), or immunofluorescence (e.g., intracellular proteins, or cell surface proteins). Non-limiting examples of cell-level phenotyping that includes functional characterization may include placing cells in different vessels for one or more different cell migration assays (such as Borden chamber assay, chemotaxis cell migration assay, or haptotaxis cell migration assay), or cell invasion assay (e.g., millicell migration assay, in which a cell's migration is recorded over time and measured).

Further details regarding example components and example operations of system 100 now will be provided.

### Training of Deep Learning Model of HFM

The deep learning model of the human foundation model may be trained using a plurality of cell images from different types of biological samples and thus, be able to detect differences in cell morphology without labeled training data. In some examples, the deep learning model 120 of the human foundation model 160 may be trained using any suitable number of images of cells, for example between about 1 and about 1.000, between about 1 and about 10,000, between about 1 and about 100,000, between about 1 and about 1,000,000, or between about 1 and about 10,000,000 images of cells. Any range of the number of cell images as training dataset may be contemplated, for example from about 1 through any number greater than about 10,000,000. As one example, the deep learning model 120 of the human foundation model is trained using a training dataset that includes at least about 10,000 images of cells—e.g., at least about 100,000 images of cells, at least about 1,000,000 images of cells, at least about 5,000,000 images of cells, at least about 10,000,000 images of cells, at least about 100,000,00 images of cells, at least about 1 billion, or more, images of cells. For example, the deep learning model 120 may be trained using between about 5,000,000 and about 1 billion images of cells. The training set may include, may consist of, or may consist essentially of (and in some examples may consist of), images of cells that are not physically stained and that are not computationally labeled in any manner. As such, in some examples the deep learning model 120 learns to recognize features from the cell images in a self-supervised manner.

In some examples, the human foundation model may comprise parameters in a range of between about 1 and about 1.000, between about 1 and about 10,000, between about 1 and about 100,000, between about 1 and about 1,000,000, between about 1 and about 10,000,000, between about 1 and about 100,000,000, or between about 1 and about 500,000,000. Any range of the number of parameters may be contemplated, for example from 1 through any number greater than 500,000,000. Some or all of the parameters may be optimized during the training process. For example, a neural network may include millions or billions of floating-point numbers connected by mathematical operations. These numbers in some instances may be called “parameters” or “weights”. In some examples, parameters are adjusted (“trained”) to transform an image of a cell into a vector (for example classification probabilities, or feature vector, depending on the use-case of the neural network). In some examples, a neural network for computer vision applications such as provided herein may have a number of parameters ranging from 1 million to upwards of 10 billion.

In some examples, the deep learning model (e.g., backbone model) of the human foundation model, which extracts image features, may be based on a convolutional neural network architecture, a vision transformer architecture, or both. The training process may apply a self-supervised learning approach that learns image features without labels and generate deep learning embeddings (vectors) that are orthogonal to each other and orthogonal to morphometric features. As used herein, embeddings that are “orthogonal” may be perpendicular to another embedding vector or set of embedding vectors. For example, vectors are considered to be orthogonal to each other if they are at right angles in n-dimensional space, where n is the size or number of elements in each vector. In some examples, “orthogonal” embeddings may have a covariance of about 0 and may be perfectly or completely orthogonal (e.g., have exactly a covariance of 0) or be substantially orthogonal with a covariance that is greater than but close to 0. In some examples, “orthogonal” embeddings include features that are “independent” of one another, meaning, that the presence or absence of one feature does not affect the presence or absence of any of the other feature. For example, a vector is orthogonal if the dot product with another vector is zero.

### Examples of Machine Learning Models for Feature Extraction

In some examples, analysis of imaging data as disclosed herein (e.g., particle imaging data, such as cell imaging data) may be performed using artificial intelligence, such as one or more machine learning algorithms.

The machine learning model (e.g., a metamodel) may be trained by using a learning model and applying learning algorithms (e.g., machine learning algorithms) on a training dataset (e.g., a dataset comprising unlabeled cell images). In some examples, given a set of training examples/cases, each marked for belonging to a specific class (e.g., specific cell type or class), a training algorithm may build a machine learning model capable of assigning features within images of cells into one category or the other, e.g., to make the model a non-probabilistic machine learning model. In some examples, the machine learning model may be used to create a new category to assign new examples/cases into the new category. In some examples, a machine learning model may be the actual trained model that is generated based on the training model.

The machine learning algorithm as disclosed herein may be configured to extract one or more morphological features of a cell from the image data of the cell. The machine learning algorithm may form a new data set based on the extracted morphological features, and the new data set need not contain the original image data of the cell. In some examples, replicas of the original images in the image data may be stored in a database disclosed herein, e.g., prior to using any of the new images for training, e.g., to keep the integrity of the images of the image data. In some examples, processed images of the original images in the image data may be stored in a database disclosed herein during or subsequent to the classifier training. In some examples, any of the newly extracted morphological features as disclosed herein may be utilized as new molecular markers for a cell or population of cells of interest to the user. A cell analysis platform as disclosed herein may be operatively coupled to one or more databases comprising non-morphological data of cells processed (e.g., genomics data, transcriptomics data, proteomics data, metabolomics data), a selected population of cells exhibiting the newly extracted morphological feature(s) may be further analyzed by their non-morphometric features to identify proteins or genes of interest that are common in the selected population of cells but not in other cells, thereby determining such proteins or genes of interest to be new molecular markers that may be used to identify such selected population of cells.

Non-limiting examples of machine learning algorithms for training a machine learning model may include supervised learning, unsupervised learning, semi-supervised learning, reinforcement learning, self-learning (also referred to as self-supervised learning), feature learning, anomaly detection, association rules, etc. In some examples, a machine learning model may be trained by using one or more learning models on such training dataset. Non-limiting examples of learning models may include artificial neural networks (e.g., convolutional neural networks, U-net architecture neural network, etc.), backpropagation, boosting, decision trees, support vector machines, regression analysis, Bayesian networks, genetic algorithms, kernel estimators, conditional random field, random forest, ensembles of machine learning models, minimum complexity machines (MCM), probably approximately correct learning (PACT), etc.

In some examples, the neural networks are designed by the modification of neural networks such as AlexNet, VGGNet, GoogLeNet, ResNet (residual networks), DenseNet, and Inception networks. In some examples, the enhanced neural networks are designed by modification of ResNet (e.g. ResNet 18, ResNet 34, ResNet 50, ResNet 101, and ResNet 152) or inception networks. In some examples, the modification comprises a series of network surgery operations that are mainly carried out to improve including inference time and/or inference accuracy.

The neural network may be used together with a Vision Transformer. Vision Transformers and their use in encoding images are described in Dosovitskiy et al., “An image is worth 16×16 words: Transformers for image recognition at scale,” International Conference on Learning Representations (ICLR) (2021) (21 pages available at arxiv.org/abs/2010.11929), the entire contents of which are incorporated by reference herein.

The machine learning algorithm as disclosed herein may utilize one or more clustering algorithms to determine that objects (e.g., features) in the same cluster may be more similar (in one or more morphological features) to each other than those in other clusters. Non-limiting examples of the clustering algorithms may include, but are not limited to, connectivity models (e.g., hierarchical clustering), centroid models (e.g. K-means algorithm), distribution models (e.g., expectation-maximization algorithm), density models (e.g., density-based spatial clustering of applications with noise (DBSCAN), ordering points to identify the clustering structure (OPTICS)), subspace models (e.g., biclustering), group models, graph-based models (e.g., highly connected subgraphs (HCS) clustering algorithms), single graph models, and neural models (e.g., using unsupervised neural network). The machine learning algorithm may utilize a plurality of models, e.g., in equal weights or in different weights. In some examples, the graph-based models may include graph-based clustering algorithms that use modularity, e.g., such as described in the following references, the entire contents of each of which are incorporated by reference herein: Blondel et al., “Fast unfolding of communities in large networks,” Journal of Statistical Mechanics: Theory and Experiment 2008: P10008 (2008); and Traag et al., “From Louvain to Leiden: guaranteeing well-connected communities,” Scientific Reports 9:5233, 12 pages, (2019).

In some examples, unsupervised and self-supervised approaches may be used to expedite labeling of image data of cells (extract features from cells). For the example of unsupervised, an embedding for a cell image may be generated. For example, the embedding may be a representation of the image in a space with reduced dimensions than the original image data. Such embeddings may be used to cluster images that are similar to one another. Thus, the labeler may be configured to batch-label the cells and increase the throughput as compared to manually labeling one or more cells.

In some examples, for the example of self-supervised learning, additional meta information (e.g., additional non-morphological information) about the sample (e.g., what disease is known or associated with the patient who provided the sample) may be used for labeling of image data of cells.

In some examples, embedding generation may use a neural net trained on predefined cell types. To generate the embeddings described herein, an intermediate layer of the neural net that is trained on predetermined image data (e.g., image data of known cell types and/or states) may be used.

In some examples, by providing enough diversity in image data or sample data to the trained model, this method may have a benefit of providing an accurate way to cluster future cells.

In some examples, embedding generation may use neural nets trained for different tasks. To generate the embeddings described herein, an intermediate layer of the neural net that is trained for a different task (e.g., a neural net that is trained on a canonical dataset such as ImageNet). Without wishing to be bound by any particular theory, this may allow the system to focus on features that matter for image classification (e.g., edges and curves) while removing a bias that may otherwise be introduced in labeling the image data.

In some examples, autoencoders may be used for embedding generation. To generate the embeddings described herein, autoencoders may be used, in which the input and the output may be substantially the same image and the squeeze layer may be used to extract the embeddings. The squeeze layer may force the model to learn a smaller representation of the image, which smaller representation may have sufficient information to recreate the image (e.g., as the output).

In some examples, for clustering-based labeling of image data or cells, as disclosed herein, an expanding training data set may be used. With the expanding training data set, one or more revisions of labeling (e.g., manual relabeling) may be needed to, for example, avoid the degradation of model performance due to the accumulated effect of mislabeled images. Such manual relabeling may be intractable on a large scale and ineffective when done on a random subset of the data. Thus, in some examples, to systematically surface images for potential relabeling, similar embedding-based clustering may be used to identify labeled images that may cluster with members of other classes. Such examples are likely to be enriched for incorrect or ambiguous labels, which may be removed (e.g., automatically or manually).

In some examples, adaptive image augmentation may be used. In order to make the models disclosed herein more robust to artifacts in the image data, (1) one or more images with artifacts may be identified, and (2) such images identified with artifacts may be added to training pipeline (e.g., for training the model). Identifying the image(s) with artifacts may comprise: (la) while imaging cells, one or more additional sections of the image frame may be cropped, which frame(s) being expected to contain just the background without any cell; (2a) the background image may be checked for any change in one or more characteristics (e.g., optical characteristics, such as brightness); and (3a) flagging/labeling one or more images that have such change in the characteristic(s). Adding the identified images to training pipeline may comprise: (2a) adding the one or more images that have been flagged/labeled as augmentation by first calculating an average feature of the changed characteristic(s) (e.g., the background median color); (2b) creating a delta image by subtracting the average feature from the image data (e.g., subtracting the median for each pixel of the image); and (3c) adding the delta image to the training pipeline.

In any of the methods or platforms disclosed herein, the model(s) may be validated (e.g., for the ability to demonstrate accurate cell classification performance). Non-limiting examples of validation metrics that may be utilized may include, but are not limited to, threshold metrics (e.g., accuracy, F-measure, Kappa, Macro-Average Accuracy, Mean-Class-Weighted Accuracy, Optimized Precision, Adjusted Geometric Mean, Balanced Accuracy, etc.), the ranking methods and metrics (e.g., receiver operating characteristics (ROC) analysis or “ROC area under the curve (ROC AUC)”), and the probabilistic metrics (e.g., root-mean-squared error). For example, the model(s) may be determined to be balanced or accurate when the ROC AUC is greater than about 0.5—e.g., greater than about 0.55, greater than about 0.6, greater than about 0.65, greater than about 0.7, greater than about 0.75, greater than about 0.8, greater than about 0.85, greater than about 0.9, greater than about 0.91, greater than about 0.92, greater than about 0.93, greater than about 0.94, greater than about 0.95, greater than about 0.96, greater than about 0.97, greater than about 0.98, greater than about 0.99, or higher.

As noted further above, the output of the machine learning encoder (model) may include, may consist of, or may consist essentially of, at least one multidimensional feature vector (which may also be referred to herein as an embedding). Elements of the vector(s) for a given image may correspond to the values of respective features that the machine learning encoder extracted from that image. Table 1 below describes example machine learning dimensions (for example, deep learning dimensions), which correspond to different features that the machine learning encoder extracts from images. In some examples, the machine learning encoder extracts n ML-based features from each image (where n is a positive integer), and outputs an array of length n, which array may be considered to be an n-dimensional vector. For the illustrative machine learning dimensions listed in Table 1, the output of the deep learning encoder may have the format:
[V₁ V₂ V₃ . . . Vₙ],
   where the subscripts 1 . . . n correspond to the respective deep learning dimension numbers, and wherein the letter V represents the value of the feature in that image that the deep learning encoder calculated. The value of n may be in any suitable range, e.g., may be between about 5 and about 1000—e.g., between about 10 and about 500, between about 50 and about 100, or range in between any of the aforementioned values. In the nonlimiting example shown in Table 1, n is equal to 64.

TABLE 1
Example Deep Learning derived features generated
using the human foundation model

| Deep learning | |
|---|---|
| dimension number | Description |
| HFMv1:DL001 | 1^{st} quantitative description of cell features |
| | derived from the HFM model |
| HFMv1:DL002 | 2^{nd} quantitative description of cell features |
| | derived from the HFM model |
| . . . | . . . |
| HFMv1:DL064 | 64^{th} quantitative description of cell features |
| | derived from the HFM model |

In one example, the ML-based features are not human-interpretable. In one example, because the ML-based features are identified using machine learning, AI, or both, the features are not human-interpretable. For example, the elements of the vector generated by the machine learning encoder may have numeric values, such as [0.1 4 2.3 . . . 10], that correspond to the quantitative “amount” of certain features that the machine learning encoder has identified as being present or not in a given image. However, in some examples it may not be possible to identify the meaning of these features, for example as they may correspond to features that are identified by neurons of a convolutional neural network and it is not possible in these examples to reconstruct what those neurons considered to be a feature.

### Computer Vision Model of HFM

The computer vision model of the human foundation model may include a set of rules to identify cell morphometric features within an image, and to encode those features into a multidimensional feature vector. In some examples, the rules may be human defined, and may correspond to features that may be understood by a human.

As noted further above, the output of the computer vision encoder (model) may include, may consist of, or may consist essentially of, at least one multidimensional feature vector (which may also be referred to herein as an embedding). Elements of the vector(s) for a given image may correspond to the values of respective features that the computer vision encoder extracted from that image. Because the features are human defined, the features may be human-interpretable. Table 2 below describes example computer vision dimensions (morphometric features), which correspond to different features that the computer vision encoder may extract from images.

From Table 2, it may be understood that morphometric features may be categorized into different groups. For example, cell morphometric features may be selected from the group consisting of position features, cell shape features, pixel intensity features, texture features, and focus features. In some examples, position features may be selected from the group consisting of: centroid X axis and centroid Y axis, where Table 2 provides respective descriptions for such features. In some examples, cell shape features may be selected from the group consisting of: area, perimeter, maximum caliper distance, minimum caliper distance, maximum radius, minimum radius, long ellipse axis, short ellipse axis, ellipse elongation, ellipse similarity, roundness, circle similarity, and convex shape, where Table 2 provides respective example descriptions for such features. In some examples, pixel intensity features are selected from the group consisting of: mean pixel intensity, standard deviation of pixel intensity, pixel intensity 25th percentile, pixel intensity 75th percentile, positive fraction, and negative fraction, where Table 2 provides respective example descriptions for such features. In some examples, texture features may be selected from the group consisting of: small set of connected bright pixels, integral; small set of connected dark pixels, integral; large set of connected bright pixels, integral; large set of connected dark pixels, integral; image moments; local binary patterns-center; local binary patterns-periphery; image sharpness; image focus; ring width; and ring intensity.

In some examples, the computer vision encoder extracts m morphometric features from each image (where m is a positive integer), and outputs an array of length m, which array may be considered to be an m-dimensional vector. For the illustrative computer vision dimensions listed in Table 2, the output of the computer vision encoder may have the format:
[W₁ W₂ W₃ . . . Wₘ],
   where the subscripts 1 . . . m correspond to the respective computer vision dimension numbers, and wherein the letter W represents the value of the feature in that image that the computer vision encoder calculated. The value of m may be in any suitable range, e.g., may be between about 5 and about 1000, e.g., between about 10 and about 500, e.g., between about 50 and about 100. In the nonlimiting example shown in Table 2, m is equal to 51.

Because the morphometric features represent features that are visible by both human and computer vision, the features may be human-interpretable. For example, the elements of the vector generated using the computer vision encoder may have numeric values, such as [5 0.8 1.4 . . . 3.7], that correspond to the quantitative “amount” of certain features that the computer vision encoder has identified as being present or not in a given image. The meanings of these features also may be understood by a human. For example, based on Table 2 it may be understood that the value of the first element (e.g., 5) is the centroid X axis in μm of the image (meaning the X axis position of the cell relative to the camera's field of view), the value of the third element (e.g., 1.4) is the cell area in μm², and so on.

The computer vision encoder may be implemented using any suitable combination of hardware and software. As one example, the system component which is implementing the HFM may include a processor and a non-volatile computer-readable medium that includes instructions for causing the processor to respectively process cell images using a computer vision encoder. The computer vision encoder may be configured to quantify the characteristics (e.g., to measure dimensions or intensities) of different features within respective cell images, and to output a vector the dimensions (elements) of which correspond to the measured values of those respective characteristics.

### Encoding ML-Based Features and Cell Morphometric Features

As noted elsewhere herein, the set of ML-based features extracted using the machine learning encoder and the set of cell morphometric features extracted using the computer vision encoder may be used to respectively encode the set of ML-based features and the set of cell morphometric features into a plurality of multi-dimensional feature vectors that represent morphology of a cell in a cell image. In examples in which the machine learning encoder extracts n ML-based features, and the computer-vision encoder extracts m cell morphological features, the multi-dimensional feature vectors may have n+m dimensions, where n and m are positive integers. Within each of the multi-dimensional feature vectors, each dimension of the n+m dimensions may be an element of that multi-dimensional feature vector, e.g., a numeric value. As one example, continuing with the example provided above, the ML-based features and the cell morphometric features may be concatenated to generate a multi-dimensional feature vector having the format:
[V₁ V₂ V₃ . . . Vₙ W₁ W₂ W₃ . . . Wₘ],
   where, similarly as above, the subscripts 1 . . . n correspond to the respective deep learning dimension numbers, the letter V represents the value of the feature in that image that the deep learning encoder calculated, the subscripts 1 . . . m correspond to the respective computer vision dimension numbers, and the letter W represents the value of the feature in that image that the computer vision encoder calculated. Note that the value of m may be the same as the value of n, in which example the plurality of multi-dimensional feature vectors extracted using the machine learning encoder and the computer vision encoder may include a same number of each of the ML-based features and the cell morphological features. In another example, the value of m may be different than the value of n, in which example the plurality of multi-dimensional feature vectors extracted using the machine learning encoder and the computer vision encoder may include a different number of each of the ML-based features and the cell morphological features. It will further be appreciated that in some examples, an array of length (n+m), or a vector of length (n+m), may be interpreted as being a plurality of multi-dimensional feature vectors, each such vector having one or more elements.

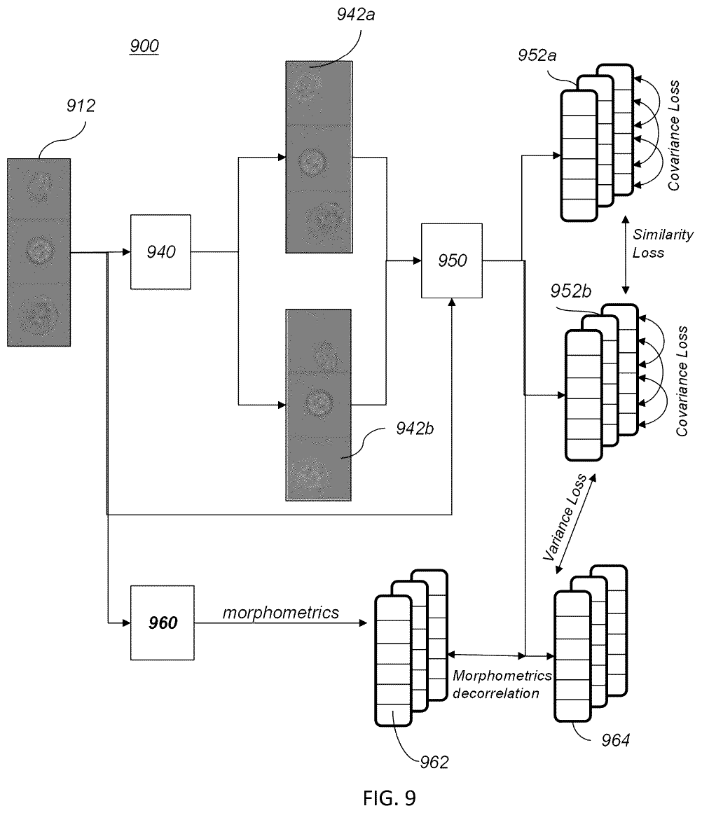

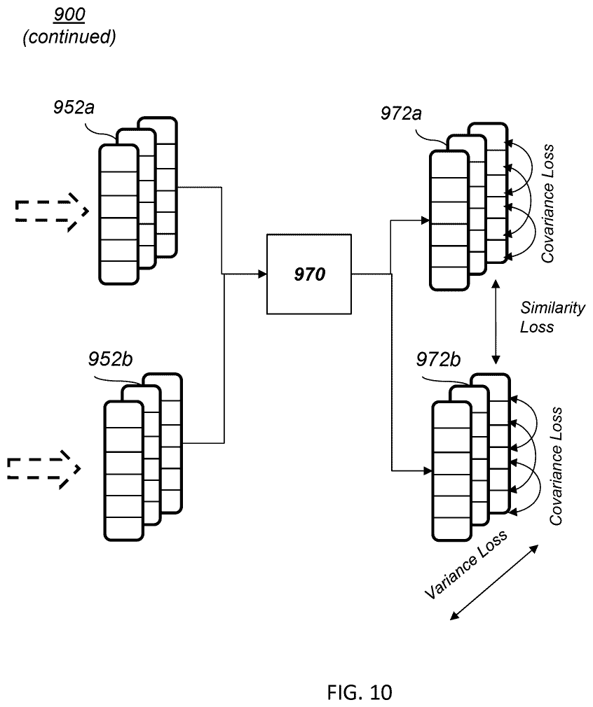

In one example, in a manner such as noted further above, the ML-based features may be orthogonal to one another as explained more particularly with reference to FIGS. 9-10. By this, it is meant that the ML-based features may all be different than one another, and may all be uncorrelated to one another. For example, ML-based feature V₁ may be different than, and uncorrelated to, each of ML-based features V₂ . . . Vₙ. In some other examples, the ML-based features may be orthogonal to the cell morphological features.

### Cell Classification

In some examples, analysis of imaging data as disclosed herein (e.g., particle imaging data, such as cell imaging data) may be performed using artificial intelligence, such as one or more machine learning algorithms. In some examples, one or more machine learning models may be used to automatically sort or categorize particles (e.g., cells) in the imaging data into one or more classes (e.g., one or more physical characteristics or morphological features, as used interchangeably herein). In some examples, cell imaging data may be analyzed using the machine learning algorithm(s) to classify (e.g., sort) a cell (e.g., a single cell) in a cell image or video. In some examples, cell imaging data may be analyzed using the machine learning algorithm(s) to determine a focus score of a cell (e.g., a single cell) in a cell image or video. In some examples, cell imaging data may be analyzed using the machine learning algorithm(s) to determine a relative distance between (i) a first plane of cells exhibiting first similar physical characteristic(s) and (ii) a second plane of cells exhibiting second similar physical characteristic(s), which first and second planes denote fluid streams flowing substantially parallel to each other in a channel. In some examples, one or more cell morphology maps as disclosed herein may be used to train one or more machine learning models (e.g., at least about 1, for example at least about 2, at least about 3, at least about 4, at least about 5, at least about 6, at least about 7, at least about 8, at least about 9, at least about 10, or more machine learning models) as disclosed herein. Each machine learning model may be trained to analyze one or more images of a cell (e.g., to extract one or more morphological features of the cell) and categorize (or classify) the cell into one or more determined class or categories of a cell (e.g., based on a type of state of the cell). In another example, the machine learning model may be trained to create a new category to categorize (or classify) the cell into the new category, e.g., when determining that the cell is morphologically distinct than any pre-existing categories of other cells.

In some examples, the entire process of cell focusing as disclosed herein (e.g., partitioning of cells into one or more planar currents flowing through the channel) may be accomplished based on de novo AI-mediated analysis of each cell (e.g., using analysis of one or more images of each cell using machine learning algorithm). This may be a complete AI or a full AI approach for cell sorting and analysis. In another example, a hybrid approach may be utilized, wherein AI-mediated analysis may analyze cells and one or more heterologous markers that are co-partitioned with the cells (e.g., into the same planar current flowing through the channel), confirm or determine the co-partitioning, after which a more conventional approach (e.g., imaging to detect presence of the heterologous markers, such as fluorescent imaging) may be utilized to sort a subsequent population of cells and the heterologous markers that are co-partitioned into the same planar current.

The machine learning model (e.g., a metamodel) may be trained by using a learning model and applying learning algorithms (e.g., machine learning algorithms) on a training dataset (e.g., a dataset comprising examples of specific classes). In some examples, given a set of training examples/cases, each marked for belonging to a specific class (e.g., specific cell type or class), a training algorithm may build a machine learning model capable of assigning new examples/cases (e.g., new datapoints of a cell or a group of cells) into one category or the other, e.g., to make the model a non-probabilistic machine learning model. In some examples, the machine learning model may be capable of creating a new category to assign new examples/cases into the new category. In some examples, a machine learning model may be the actual trained model that is generated based on the training model.

The machine learning algorithm as disclosed herein may be configured to extract one or more morphological features of a cell from the image data of the cell. The machine learning algorithm may form a new data set based on the extracted morphological features, and the new data set need not contain the original image data of the cell. In some examples, replicas of the original images in the image data may be stored in a database disclosed herein, e.g., prior to using any of the new images for training, e.g., to keep the integrity of the images of the image data. In some examples, processed images of the original images in the image data may be stored in a database disclosed herein during or subsequent to the classifier training. In some examples, any of the newly extracted morphological features as disclosed herein may be utilized as new molecular markers for a cell or population of cells of interest to the user. As cell analysis platform as disclosed herein may be operatively coupled to one or more databases comprising non-morphological data of cells processed (e.g., genomics data, transcriptomics data, proteomics data, metabolomics data), a selected population of cells exhibiting the newly extracted morphological feature(s) may be further analyzed by their non-morphometric features to identify proteins or genes of interest that are common in the selected population of cells but not in other cells, thereby determining such proteins or genes of interest to be new molecular markers that may be used to identify such selected population of cells.

The machine learning algorithm as disclosed herein may utilize one or more clustering algorithms to determine that objects (e.g., cells) in the same cluster may be more similar (in one or more morphological features) to each other than those in other clusters. Non-limiting examples of the clustering algorithms may include, but are not limited to, connectivity models (e.g., hierarchical clustering), centroid models (e.g. K-means algorithm), distribution models (e.g., expectation-maximization algorithm), density models (e.g., density-based spatial clustering of applications with noise (DBSCAN), ordering points to identify the clustering structure (OPTICS)), subspace models (e.g., biclustering), group models, graph-based models (e.g., highly connected subgraphs (HCS) clustering algorithms), single graph models, and neural models (e.g., using unsupervised neural network). The machine learning algorithm may utilize a plurality of models, e.g., in equal weights or in different weights.

In some examples, for the example of self-supervised learning, additional meta information (e.g., additional non-morphological information) about the sample (e.g., what disease is known or associated with the patient who provided the sample) may be used for labeling of image data of cells.

In some examples, embedding generation (e.g., see FIGS. 9-10 and 16-17) may use a neural net trained on predefined cell types. To generate the embeddings described herein, an intermediate layer of the neural net that is trained on predetermined image data (e.g., image data of known cell types and/or states) may be used. By providing enough diversity in image data/sample data to the trained model, this method may provide an accurate way to cluster future cells.

In some examples, embedding generation may use neural nets trained for different tasks. To generate the embeddings described herein, an intermediate layer of the neural net that is trained for a different task (e.g., a neural net that is trained on a canonical dataset such as ImageNet). Without wishing to be bound by any particular theory, this may allow to focus on features that matter for image classification (e.g., edges and curves) while removing a bias that may otherwise be introduced in labeling the image data.

In some examples, for clustering-based labeling of image data or cells, as disclosed herein, an expanding training data set may be used. With the expanding training data set, one or more revisions of labeling (e.g., manual relabeling) may be needed to, e.g., avoid the degradation of model performance due to the accumulated effect of mislabeled images. Such manual relabeling may be intractable on a large scale and ineffective when done on a random subset of the data. Thus, to systematically surface images for potential relabeling, for example, similar embedding-based clustering may be used to identify labeled images that may cluster with members of other classes. Such examples are likely to be enriched for incorrect or ambiguous labels, which may be removed (e.g., automatically or manually).

### System for Cell Morphology Analysis

Cell morphology may be highly indicative of a cell's phenotype and function, but it is also highly dynamic and complex. Traditional analysis of cell morphology by human eyes has significant limitations. Other methods of assessing and characterizing cell morphology are also limited to imaging or sorting with cell labels.

Some examples of the present disclosure provide a quantitative, high-dimensional, unbiased platform to assess cell morphology and magnify insights into a cell's phenotype and functions. In some examples, the system as described herein may provide imaging of single cells and label-free sorting in one platform. For example, the system may directly capture high-resolution brightfield images of cells in real time. The system may also enable cell sorting based on their morphology without involving any cell labels. The cells may remain viable and minimally perturbed after the sorting process. In addition, the system may allow collection of sorted cells for downstream analysis, for example, single-cell RNA sequencing.

The system may comprise, or be compatible with, the human foundation model for high-dimensional morphological feature analysis. In some examples, the system may comprise or be compatible with a data suite that may allow users to store, visualize, and analyze images and high-dimensional data. Hence, the system may enable the end-to-end process including cell imaging, morphology analysis, sorting, and classification. In some examples, the system may comprise a microfluidic platform. When cells flow through the microfluidic platform, the system may capture high-resolution brightfield images of each individual cell. The images may be processed by the human foundation model for extracting high-dimensional features corresponding to the cells. The system may sort the cells in different categories, based on the distinct morphological features. The imaging, single-cell morphology analysis, sorting, and classification may occur in real time.

As described above, FIG. 1 illustrates system 100 which includes, and illustrates the interaction between, a microfluidics platform (e.g., corresponding to any microfluidics platform of this disclosure) including microfluidic chip 110, the human foundation model 180 (e.g., a deep learning model and a computer-vision model), and a data suite 190, in accordance with some examples of the present disclosure. The microfluidics platform (e.g., REM-I) may include or be compatible with the human foundation model 180 and the data suite 190. Example interactions between the microfluidics platform, the human foundation model 180, and the data suite 190 will be described in further detail elsewhere herein. In some examples, brightfield images of single cells are analyzed in real-time by human foundation model 180 to generate quantitative AI embeddings that may include reproducible high-dimensional descriptions of cell morphology. In some examples, morphologically distinct cell groups may be sorted in real-time by system 100 for downstream analysis, including up to approximately 6 populations per run.

In some examples, system 100 may be used in a workflow starting from preparing and loading cells onto the microfluidics platform. In some examples of such an operation, samples of cells (e.g., in single cell suspension) are loaded onto a microfluidic chip 110. In some examples, the preparation of samples may comprise dissociation of cells into a single-cell suspension and loading the suspension onto the microfluidics platform. Subsequently, the system 100 may capture images of the cells, and the human foundation model 180 may characterize the cells in real time as they flow through the microfluidic chip 110. In some examples, images of single cells are captured and analyzed in real-time by the human foundation model 180 to generate multi-dimensional quantitative morphological profiles. The human foundation model 180 may process the images of the cells and generate high-dimensional features reflecting the cell morphology. The images and extracted features may be stored in the data suite 190. The data suite 190 also visualizes the cell morphology data by, for example, generating user-defined cell clusters based on cell types. The data suite 190 may also provide in-depth data analysis, including selecting cell populations of interest to sort on the microfluidics platform. The system may recover sorted cells in a plurality of collection wells, e.g., in a manner such as described with reference to FIG. 2, which may be used for downstream analyses. In addition, the collected morphology data (referred to as embeddings) may be further analyzed as a unique modality, and users may continuously train customized models for specific applications. Tables 3 and 4, provided further below, list example parameters, specifications, and components of system 100.

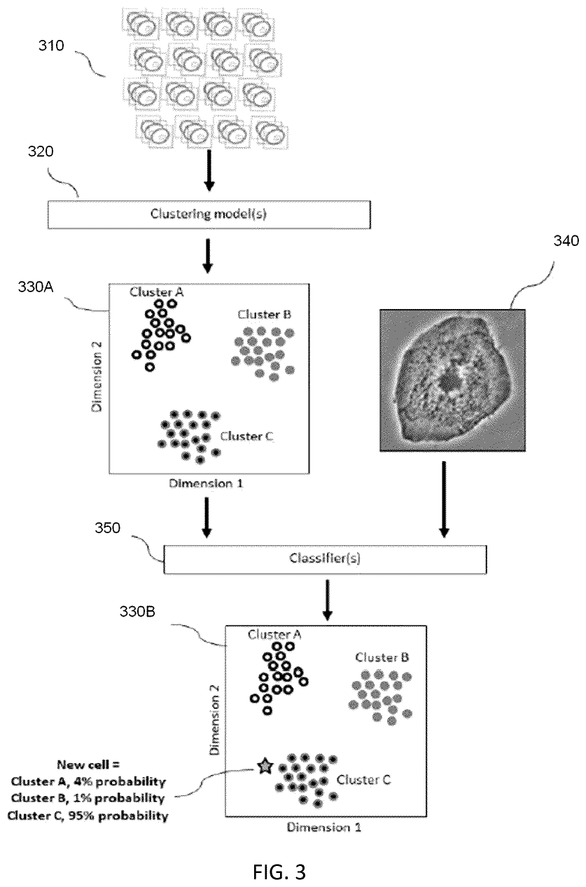

FIG. 3 schematically illustrates an example method for classifying a cell. The method may comprise processing image data 310 comprising tag-free images/videos of single cells (e.g., image data 310 consisting of tag-free images/videos of single cells). Various clustering analysis models 320 as disclosed herein may be used to process the image data 310 to extract one or more morphometric features of the cells from the image data 310, and generate a cell morphology map 330A based on the extracted one or more morphometric features. For example, the cell morphology map 330A may be generated based on two morphometric features as dimension 1 and dimension 2. The cell morphology map 330A may comprise one or more clusters (e.g., clusters A, B, and C) of datapoints, each datapoint representing an individual cell from the image data 310. The cell morphology map 330A and the clusters A-C therein may be used to train classifier(s) 350. Subsequently, a new image 340 of a new cell may be obtained and processed by the trained classifier(s) 350 to automatically extract and analyze one or more morphometric features from the cellular image 340 and plot it as a datapoint on the cell morphology map 330A. Based on its proximity, correlation, or commonality with one or more of the morphologically-distinct clusters A-C on the cell morphology map 330A, the classifier(s) 350 may automatically classify the new cell. The classifier(s) 350 may determine a probability that the cell in the new image data 340 belongs to cluster C (e.g., the likelihood for the cell in the new image data 340 to share one or more commonalities and/or characteristics with cluster C more than with other clusters A/B). For example, the classifier(s) 350 may determine and report that the cell in the new image data 340 has a 95% probability of belonging to cluster C, 1% probability of belonging to cluster B, and 4% probability of belong to cluster A, solely based on analysis of the tag-free image 340 and one or more morphological features of the cell extracted therefrom.

An image and/or video (e.g., a plurality of images and/or videos) of one or more cells as disclosed herein (e.g., that of image data 310 in FIG. 3) may be captured while the cell(s) is suspended in a fluid (e.g., an aqueous liquid, such as a buffer) and/or while the cell(s) is moving (e.g., transported across a microfluidic channel). For example, the cell need not be suspended is a gel-like or solid-like medium. The fluid may comprise a liquid that is heterologous to the cell(s)'s natural environment. For example, cells from a subject's blood may be suspended in a fluid that comprises (i) at least a portion of the blood and (ii) a buffer that is heterologous to the blood. The cell(s) may be not immobilized (e.g., embedded in a solid tissue or affixed to a microscope slide, such as a glass slide, for histology) or adhered to a substrate. The cell(s) may be isolated from the natural environment or niche (e.g., a part of the tissue the cell(s) it would be in if not retrieved from a subject by human intervention) when the image and/or video of the cell(s) is captured. For example, the image and/or video need not be from a histological imaging. The cell(s) need not be sliced or sectioned prior to obtaining the image and/or video of the cell, and, as such, the cell(s) may remain substantially intact as a whole during capturing of the image and/or video.

When the image data is processed, e.g., to extract one or more morphological features of a cell, each cell image may be annotated with the extracted one or more morphological features and/or with information that the cell image belongs to a particular cluster (e.g., a probability).

The cell morphology map may be a visual (e.g., graphical) representation of one or more clusters of datapoints. The cell morphology map may be a 1-dimensional (1D) representation (e.g., based on one morphometric feature as one parameter or dimension) or a multi-dimensional representation, such as a 2-dimensional (2D) representation (e.g., based on two morphometric features as two parameters or dimensions), a 3-dimensional (3D) representation (e.g., based on three morphometric features as three parameters or dimensions), a 4-dimensional (4D) representation, etc. In some examples, one morphometric feature of a plurality of morphometric features used for blotting the cell morphology map may be represented as a non-axial parameter (e.g., non-x, y, or z axis), such as, distinguishable colors (e.g., heatmap), numbers, letters (e.g., texts of one or more languages), and/or symbols (e.g., a square, oval, triangle, square, etc.). For example, a heatmap may be used as colorimetric scale to represent the classifier prediction percentages for each cell against a cell class, cell type, or cell state.

The cell morphology map may be generated based on one or more morphological features (e.g., characteristics, profiles, fingerprints, etc.) from the processed image data. Non-limiting examples of one or more morphometric features of a cell, as disclosed herein, that may be extracted from one or more images of the cell may include, but are not limited to (i) shape, curvature, size (e.g., diameter, length, width, circumference), area, volume, texture, thickness, roundness, etc. of the cell or one or more components of the cell (e.g., cell membrane, nucleus, mitochondria, etc.), (ii) number or positioning of one or more contents (e.g., nucleus, mitochondria, etc.) of the cell within the cell (e.g., center, off-centered, etc.), and (iii) optical characteristics of a region of the image(s) (e.g., unique groups of pixels within the image(s)) that correspond to the cell or a portion thereof (e.g., light emission, transmission, reflectance, absorbance, fluorescence, luminescence, etc.).

Non-limiting examples of clustering as disclosed herein may be hard clustering (e.g., determining whether a cell belongs to a cluster or not), soft clustering (e.g., determining a likelihood that a cell belongs to each cluster to a certain degree), strict partitioning clustering (e.g., determining whether each cell belongs to exactly one cluster), strict partitioning clustering with outliers (e.g., determining whether a cell may also belong to no cluster), overlapping clustering (e.g., determining whether a cell may belong to more than one cluster), hierarchical clustering (e.g., determining whether cells that belong to a child cluster may also belong to a parent cluster), and subspace clustering (e.g., determining whether clusters are not expected to overlap).

Cell clustering and/or generation of the cell morphology map, as disclosed herein, may be based on a single morphometric feature of the cells. In another example, cell clustering and/or generation the cell morphology map may be based on a plurality of different morphometric features of the cells. In some examples, the plurality of different morphometric features of the cells may have the same weight or different weights. A weight may be a value indicative of the importance or influence of each morphometric feature relative to one another in training the classifier or using the classifier to (i) generate one or more cell clusters, (ii) generate the cell morphology map, or (iii) analyze a new cellular image to classify the cellular image as disclosed herein. For example, cell clustering may be performed by having 50% weight on cell shape, 40% weight on cell area, and 10% weight on texture (e.g., roughness) of the cell membrane. In some examples, the classifier as disclosed herein may be configured to adjust the weights of the plurality of different morphometric features of the cells during analysis of new cellular image data, thereby to yield a most optimal cell clustering and cell morphology map. The plurality of different morphometric features with different weights may be utilized during the same analysis operation for cell clustering and/or generation of the cell morphology map.

The plurality of different morphometric features may be analyzed hierarchically. In some examples, a first morphometric feature may be used as a parameter to analyze image data of a plurality of cells to generate an initial set of clusters. Subsequently, a second and different morphometric feature may be used as a second parameter to (i) modify the initial set of clusters (e.g., optimize arrangement among the initial set of clusters, re-group some clusters of the initial set of clusters, etc.) and/or (ii) generate a plurality of sub-clusters within a cluster of the initial set of clusters. In some examples, a first morphometric feature may be used as a parameter to analyze image data of a plurality of cells to generate an initial set of clusters, to generate a 1D cell morphology map. Subsequently, a second morphometric feature may be used as a parameter to further analyze the clusters of the 1D cell morphology map, to modify the clusters and generate a 2D cell morphology map (e.g., a first axis parameter based on the first morphometric feature and a second axis parameter based on the second morphometric feature).

In some examples of the hierarchical clustering as disclosed herein, an initial set of clusters may be generated based on an initial morphological feature that is extracted from the image data, and one or more clusters of the initial set of clusters may comprise a plurality of sub-clusters based on second morphological features or sub-features of the initial morphological feature. For example, the initial morphological feature may be cell type, such as a first type of cell (or not), and the sub-features may be different sub-types of the first type of cell, or different stages of that cell.

Each datapoint may represent an individual cell or a collection of a plurality of cells (e.g., at least about 2—e.g., at least about 3, at least about 4, at least about 5, at least about 6, at least about 7, at least about 8, at least about 9, or at least about 10 cells or more). Each datapoint may represent an individual image (e.g., of a single cell or a plurality of cells) or a collection of a plurality of images (e.g., at least about 2, for example at least about 3, at least about 4, at least about 5, at least about 6, at least about 7, at least about 8, at least about 9, or at least about 10 images of the same single cell or different cells or more). The cell morphology map may comprise at least about 1, for example or at least about 5, or at least or at least about 10, or at least about 50, or at least about 100, or at least about 500 clusters, or more. Each cluster as disclosed herein may comprise a plurality of sub-clusters, e.g., at least about 2, for example or at least about 5, or at least about 10, or at least about 50, or at least about 100, or at least about 500 sub-clusters or more. A cluster (or sub-cluster) may comprise datapoints representing cells of the same type/state. In another example, a cluster (or sub-cluster) may comprise datapoints representing cells of different types/states. A cluster (or sub-cluster) may comprise at least about 1, for example at least about 2, at least about 5, at least about 10, at least about 50, at least about 100, at least about 500, at least about 1,000, at least about 5,000, at least about 10,000, at least about 50,000, or at least about 100,000 datapoints or more.

Two or more clusters may overlap in a cell morphology map. In another example, no clusters may overlap in a cell morphology map. In some examples, an allowable degree of overlapping between two or more clusters may be adjustable (e.g., manually or automatically by a machine learning algorithm) depending on the quality, condition, or size of data in the image data being processed.

A cluster (or sub-cluster) as disclosed herein may be represented with a boundary (e.g., a solid line or a dashed line). In another example, a cluster or sub-cluster need not be represented with a boundary, and may be distinguishable from other cluster(s) sub-cluster(s) based on their proximity to one another.

A cluster (or sub-cluster) or a data comprising information about the cluster may be annotated based on one or more annotation schema (e.g., predefined annotation schema). Such annotation may be manual (e.g., by a user of the method or system disclosed herein) or automatically (e.g., by any of the machine learning algorithms disclosed herein). The annotation of the clustering may be related the one or more morphometric features of the cells that have been analyzed (e.g., cell shape, cell area, optical characteristic(s), etc.) to generate the cluster or assign one or more datapoints to the cluster. In another example, the annotation of the clustering may be related to information that has not been used or analyzed to generate the cluster or assign one or more datapoints to the cluster (e.g., genomics, transcriptomics, or proteomics, etc.). In such example, the annotation may be utilized to add additional “layers” of information to each cluster.

In some examples, an interactive annotation tool may be provided that permits one or more users to modify any process of the method described herein. For example, the interactive annotation tool may allow a user to curate, verify, edit, and/or annotate the morphologically-distinct clusters. In another example, the interactive annotation tool may process the image data, extract one or more morphological features from the image data, and allow the user to select one or more of the extracted morphological features to be used as a basis to generate the clusters and/or the cell morphology map. After the generation of the clusters and/or the cell morphology map, the interactive annotation tool may allow the user to annotate each cluster and/or the cell morphology map using (i) a predefined annotation schema or (ii) a new, user-defined annotation schema. In another example, the interactive annotation tool may allow user to assign different weights to different morphological features for the clustering and/or map plotting. In another example, the interactive annotation tool may allow user to select with imaging data (or which cells) to be used and/or which imaging data (or which cells, cell clumps, artifacts, or debris) to be discarded, for the clustering and/or map plotting. A user may manually identify incorrectly clustered cells, or the human foundation model may provide probability or correlation value of cells within each cluster and identify any outlier (e.g., a datapoint that would change the outcome of the probability/correlation value of the cluster(s) by a certain percentage value). Thus, the user may choose to move the outliers using the interactive annotation tool to further tune the cell morphology map, e.g., to yield a “higher resolution” map.

One or more cell morphology maps as disclosed herein may be used to train one or more classifiers (e.g., at least about 1, for example at least about 2, at least about 3, at least about 4, at least about 5, at least about 6, at least about 7, at least about 8, at least about 9, at least about 10, or more classifiers) as disclosed herein. Each classifier may be trained to analyze one or more images of a cell (e.g., to extract one or more morphological features of the cell) and categorize (or classify) the cell into one or more determined class or categories of a cell (e.g., based on a type of state of the cell). In another example, the classifier may be trained to create a new category to categorize (or classify) the cell into the new category, e.g., when determining that the cell is morphologically distinct than any pre-existing categories of other cells.

In a manner such as described herein, the human foundation model as disclosed herein may be configured to extract morphological features of a cell from the image data of the cell. The human foundation model may form a new data set based on the extracted morphological features, and the new data set need not contain the original image data of the cell. In some examples, replicas of the original images in the image data may be stored in a database disclosed herein, e.g., prior to using any of the new images for training, e.g., to keep the integrity of the images of the image data. In some examples, processed images of the original images in the image data may be stored in a database disclosed herein during or subsequent to the classifier training. In some examples, any of the newly extracted morphological features as disclosed herein may be utilized as new molecular markers for a cell or population of cells of interest to the user. As cell analysis platform as disclosed herein may be operatively coupled to one or more databases comprising non-morphological data of cells processed (e.g., genomics data, transcriptomics data, proteomics data, metabolomics data), a selected population of cells exhibiting the newly extracted morphological feature(s) may be further analyzed by their non-morphometric features to identify proteins or genes of interest that are common in the selected population of cells but not in other cells, thereby determining such proteins or genes of interest to be new molecular markers that may be used to identify such selected population of cells.

In some examples, unsupervised and self-supervised approaches may be used to expedite labeling of image data of cells. For the example of unsupervised, an embedding for a cell image may be generated. For example, the embedding may be a representation of the image in a space with reduced dimensions than the original image data. Such embeddings may be used to cluster images that are similar to one another. Thus, the labeler may be configured to batch-label the cells and increase the throughput as compared to manually labeling one or more cells.

In some examples, for the example of self-supervised learning, additional meta information (e.g., additional non-morphological information) about the sample (e.g., what disease is known or associated with the patient who provided the sample) may be used for labeling of image data of cells.

In some examples, embedding generation may use a neural net trained on predefined cell types. To generate the embeddings described herein, an intermediate layer of the neural net that is trained on predetermined image data (e.g., image data of known cell types and/or states) may be used. By providing enough diversity in image data/sample data to the trained model/classifier, this method may provide an accurate way to cluster future cells.

The cell morphology map as disclosed herein may comprise an ontology of the one or more morphological features. The ontology may be an alternative medium to represent a relationship among various datapoints (e.g., each representing a cell) analyzed from an image data. For example, an ontology may be a data structure of information, in which nodes may be linked by edges. An edge may be used to define a relationship between two nodes. For example, a cell morphology map may comprise a cluster comprising sub-clusters, and the relationship between the cluster and the sub-clusters may be represented in an nodes/edges ontology (e.g., an edge may be used to describe the relationship as a subclass of, genus of, part of, stem cell of, differentiated from, progeny of, diseased state of, targets, recruits, interacts with, same tissue, different tissue, etc.).

In some examples, one-to-one morphology to genomics mapping may be utilized. An image of a single cell or images of multiple “similar looking” cells may be mapped to its/their molecular profile(s) (e.g., genomics, proteomics, transcriptomics, etc.). In some examples, classifier-based barcoding may be performed. Each sorting event (e.g., positive classifier) may push the sorted cell(s) into an individual well or droplet with a unique barcode (e.g., nucleic acid or small molecule barcode). The exact barcode(s) used for that individual classifier positive event may be recorded and tracked. Following, the cells may be lysed and molecularly analyzed together with the barcode(s). The result of the molecular analysis may then be mapped (e.g., one-to-one) to the image(s) of the individual (or ensemble of) sorted cell(s) captured while the cell(s) are flowing in a flow channel (e.g., fluidic channel 113 described with reference to FIG. 1, or fluidic channel 213 described with reference to FIG. 2). In some examples, class-based sorting may be utilized. Cells that are classified in the same class based at least on their morphological features may be sorted into a single well or droplet with a pre-determined barcoded material, and the cells may be lysed, molecularly analyzed, then any molecular information may be used for the one-to-one mapping as disclosed herein.

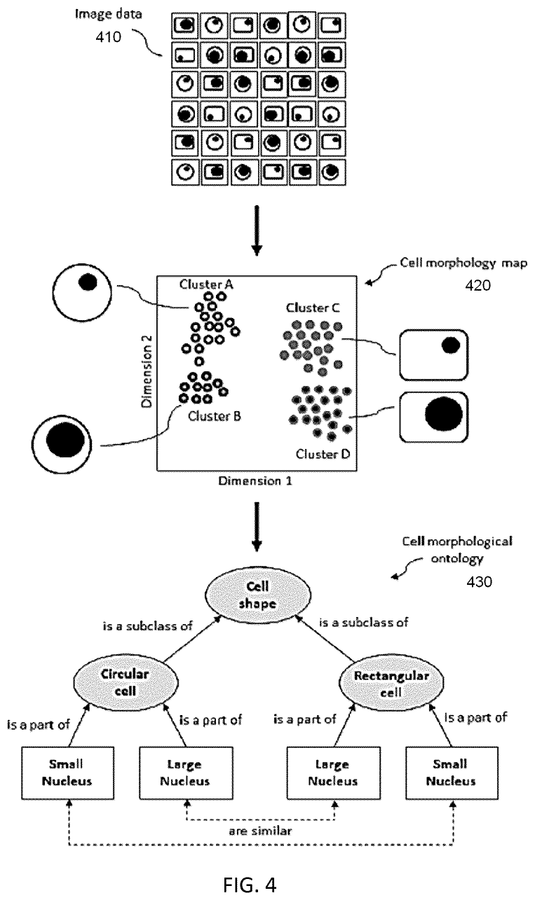

FIG. 4 schematically illustrates different ways of representing analysis data of image data of cells. Tag-free image data 410 of cells (e.g., circular cells and square cells) having different nuclei (e.g., small nucleus and large nucleus) may be analyzed by any of the methods disclosed herein (e.g., based on extraction of one or more morphological features). For example, any of the classifier(s) disclosed herein may be used to analyze and plot the image data 410 into a cell morphology map 420, comprising four distinguishable clusters: cluster A (circular cell, small nucleus), cluster B (circular cell, large nucleus), cluster C (square cell, small nucleus), and cluster D (square cell, large nucleus). The classifier(s) may also represent the analysis in a cell morphological ontology 430, in which a top node (“cell shape”) may be connected to two sub-nodes (“circular cell” and “rectangular cell”) using an edge (“is a subclass of”) to define the relationship between the nodes. Each sub-node may also connected to its own sub-nodes (“small nucleus” and “large nucleus”) using an edge (“is a part of”) to define their relationships. The sub-nodes (e.g., “small nucleus” and “large nucleus”) may also be connected using one or more edges (“are similar”) to further define their relationship.

The cell morphology map or cell morphological ontology as disclosed herein may be further annotated with one or more non-morphological data of each cell. As shown in FIG. 3, the ontology 430 from FIG. 4 may be further annotated with information about the cells that may not be extractable from the image data used to classify the cells (e.g., molecular profiles obtained using molecular barcodes, as disclosed herein). Non-limiting examples of such non-morphological data may be from additional treatment and/or analysis, including, but not limited to, cell culture (e.g., proliferation, differentiation, etc.), cell permeabilization and fixation, cell staining by a probe, mass cytometry, multiplexed ion beam imaging (MIBI), confocal imaging, nucleic acid (e.g., DNA, RNA) or protein extraction, polymerase chain reaction (PCR), target nucleic acid enrichment, sequencing, sequence mapping, etc. Examples of the probe used for cell staining (or tagging) may include, but are not limited to, a fluorescent probe (e.g., for staining chromosomes such as X, Y, 13, 18 and 21 in fetal cells), a chromogenic probe, a direct immunoagent (e.g. labeled primary antibody), an indirect immunoagent (e.g., unlabeled primary antibody coupled to a secondary enzyme), a quantum dot, a fluorescent nucleic acid stain (such as DAPI, Ethidium bromide, Sybr green, Sybr gold, Sybr blue, Ribogreen, Picogreen, YoPro-1, YoPro-2 YoPro-3, YOYo, Oligreen acridine orange, thiazole orange, propidium iodine, or Hoeste), another probe that emits a photon, or a radioactive probe. In some examples, the instrument(s) for the additional analysis may comprise a computer executable logic that performs karyotyping, in situ hybridization (ISH) (e.g., florescence in situ hybridization (FISH), chromogenic in situ hybridization (CISH), nanogold in situ hybridization (NISH)), restriction fragment length polymorphism (RFLP) analysis, polymerase chain reaction (PCR) techniques, flow cytometry, electron microscopy, quantum dot analysis, or detects single nucleotide polymorphisms (SNPs) or levels of RNA.

Analysis of the image data (e.g., extracting one or more morphological features form the image data, determining clustering and/or cell morphology map based on the image data, etc.) may be performed (e.g., automatically) within less than about 1 hour, e.g., less than about 30 minutes, or less than about 10 minutes, or less than about 5 minutes, or less than about 1 minute, or less than about 30 seconds, or less than about 10 seconds, or less than about 5 seconds, about 1 second, or less. In some examples, such analysis may be performed in real-time.

One or more morphological features utilized for generating the clusters or the cell morphology map, as disclosed herein, may be selected automatically (e.g., by one or more machine learning algorithms) or, alternatively, selected manually by a user using a user interface (e.g., graphical user interface (GUI)). The GUI may show visualization of, for example, (i) the one or more morphological parameters extracted from the image data (e.g., represented as images, words, symbols, predefined codes, etc.), (ii) the cell morphology map comprising one or more clusters, or (iii) the cell morphological ontology. The user may select, using the GUI, which morphological parameter(s) to be used to generate the clusters and the cell morphological map prior to actual generation of the clusters and the cell morphological map. The user may, upon seeing or receiving a report about the generated clusters and the cell morphological map, retroactively modify the types of morphological parameter(s) to use, thereby to (i) modify the clustering or the cell morphological mapping and/or (ii) create new cluster(s) or new cell morphological map(s). In some examples, the user may select one or more regions to be excluded or included for further analysis or further processing of the cells (e.g., sorting in the future or in real-time). For example, a microfluidic system as disclosed herein may be utilized to capture image(s) of each cell from a population of cells, and any of the methods disclosed herein may be utilized to analyze such image data to generate a cell morphology map comprising clusters representing the population of cells. The user may select one or more clusters or sub-clusters to be sorted, and the input may be provided to the microfluidic system to sort at least a portion of the cells into one or more sub-channels of the microfluidic system (e.g., in real-time) accordingly. In another example, the user may select one or more clusters or sub-clusters to be excluded during sorting (e.g., to get rid of artifacts, debris, or dead cells), and the input may be provided to the microfluidic system to sort at least a portion of the cells into one or more sub-channels of the microfluidic system (e.g., in real-time) accordingly without such artifacts, debris, or dead cells.

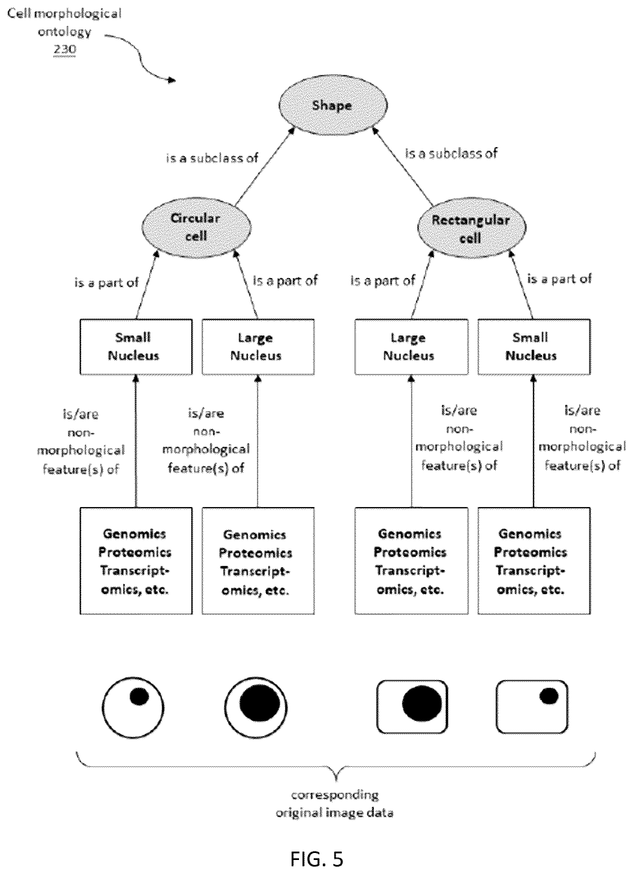

The cell morphology map or cell morphological ontology as disclosed herein may be further annotated with one or more non-morphological data of each cell. As shown in FIG. 5, the ontology 430 from FIG. 4 may be further annotated with information about the cells that may not be extractable from the image data used to classify the cells (e.g., molecular profiles obtained using molecular barcodes, as disclosed herein).

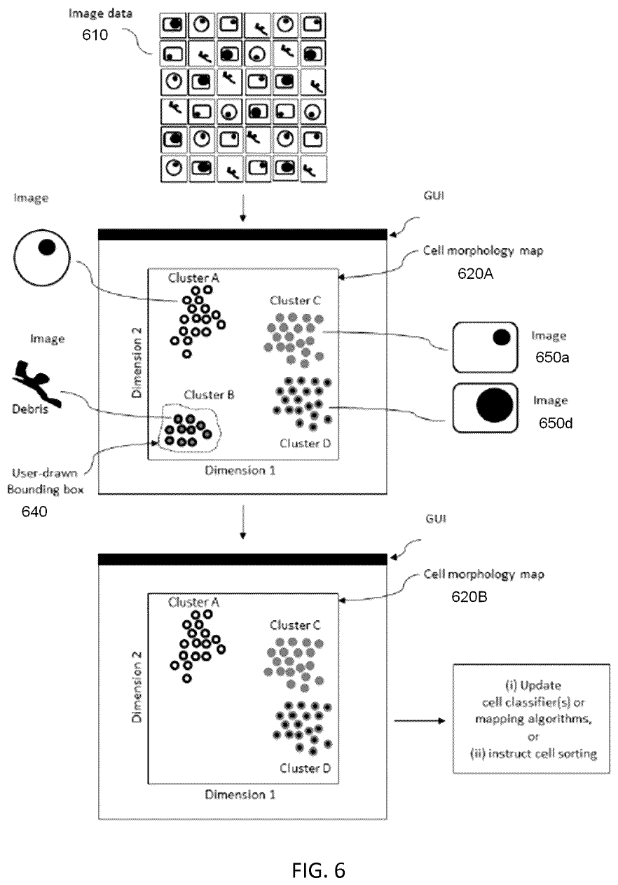

FIG. 6 schematically illustrates a method for a user to interact (e.g., using GUI) with any one of the methods disclosed herein. Image data 610 of a plurality of cells may be processed, using any one of the methods disclosed herein, to generate a cell morphology map 620A that represents the plurality of cells as datapoints in different clusters A, B, C, and D. The cell morphology map 620A may be displayed to the user using the GUI 630. The user may select each cluster or a datapoint within each cluster to visualize one or more images 650a, b, c, or d of the cells classified into the cluster. Upon visualization of the images, the user may draw a box 640 (e.g., using any user-defined shape and/or size) around one or more datapoints or around a cluster. For example, the user may draw a box 640 around a cluster of “debris” datapoints, to, e.g., remove the selected cluster and generate a new cell morphology map 620B. The user input may be used to update cell classifying algorithms, mapping algorithms, cell flowing mechanism (e.g., velocity of cells, positioning of the cells within a flow channel, adjusting imaging focal length/plane of one or more sensors/cameras of an imaging module (also referred to as an imaging device herein) that captures one or more images/videos of cells flowing through the cartridge, etc.), cell sorting mechanisms in the flow channel, cell sorting instructions in the flow channel, etc. For example, upon the user's selection, the classifier may be trained to identify one or more common morphological features within the selected datapoints (e.g., features that distinguish the selected datapoints from the unselected data). Features of the selected group may be used to further identify other cells from other samples having similar feature(s) for further analysis or discard cells having similar feature(s), e.g., for cell sorting.

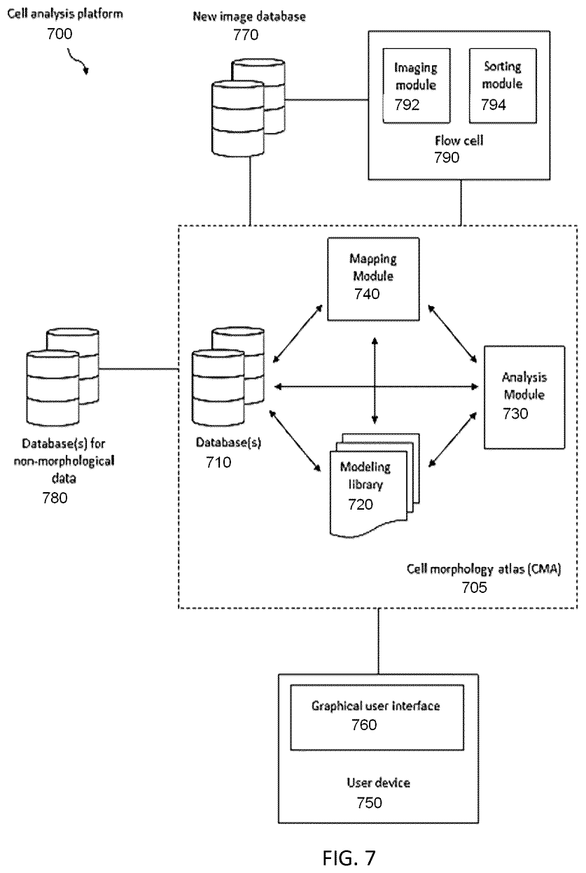

The present disclosure also describes a cell analysis platform, e.g., for analyzing or classifying a cell. The cell analysis platform may be a product of any one of the methods disclosed herein. In another example, or in addition to, the cell analysis platform may be used as a basis to execute any one of the methods disclosed herein. For example, the cell analysis platform may be used to process image data comprising tag-free images of single cells to generate a new cell morphology map of various cell clusters. In another example, the cell analysis platform may be used to process image data comprising tag-free images of single cells to compare the cell to pre-determined (e.g., pre-analyzed) images of known cells or cell morphology map(s), such that the single cells from the image data may be classified, e.g., for cell sorting. FIG. 7 illustrates an example cell analysis platform (e.g., machine learning/artificial intelligence platform) for analyzing image data of one or more cells. The cell analysis platform 700 may comprise a cell morphology atlas (CMA) 705. The CMA 705 may comprise a database 710 having a plurality of annotated single cell images that are grouped into morphologically-distinct clusters (e.g., represented a texts, as cell morphology map(s), or cell morphological ontology(ies)) corresponding to a plurality of classifications (e.g., predefined cell classes). The CMA 705 may comprise a modeling unit comprising one or more models (e.g., modeling library 720 comprising, such as, one or more machine learning algorithms disclosed herein) that are trained and validated using datasets from the CMA 705, to process image data comprising images/videos of one or more cells to identify different cell types and/or states based at least on morphological features. The CMA 705 may comprise an analysis module 730 comprising one or more classifiers as disclosed herein. The classifier(s) may use one or more of the models from the modeling library 720 to, e.g., (1) classify one or more images taken from a sample, (2) assess a quality or state of the sample based on the one or more images, (3) map one or more datapoints representing such one or more images onto a cell morphology map (or cell morphological ontology) using a mapping module 740. The CMA 705 may be operatively coupled to one or more additional database 770 to receive the image data comprising the images/videos of one or more cells. For example, the image data from the database 770 may be obtained from an imaging module 792 of a cartridge 790, which may also be operatively coupled to the CMA 705. The cartridge may direct flow of a sample comprising or suspected of comprising a target cell, and capture one or more images of contents (e.g., cells) within the sample by the imaging module 792. Any image data obtained by the imaging module 792 may be transmitted directly to the CMA 705 and/or to the new image database 770. In another example, the CMA 705 may be operatively coupled to one or more additional databases 780 comprising non-morphological data of any of the cells (e.g., genomics, transcriptomics, or proteomics, etc.), e.g., to further annotate any of the datapoint, cluster, map, ontology, images, as disclosed herein. The CMA 705 may be operatively coupled to a user device 750 (e.g., a computer or a mobile device comprising a display) comprising a GUI 760 for the user to receive information from and/or to provide input (e.g., instructions to modify or assist any portion of the method disclosed herein). Any classification made by the CMA and/or the user may be provided as an input to the sorting module 794 of the cartridge 790. Based on the classification, the sorting module may determine, for example, (i) when to activate one or more sorting mechanisms at the sorting junction of the cartridge 790 to sort one or more cells of interest, (ii) which sub-channel of a plurality of sub channels to direct each single cell for sorting. In some examples, the sorted cells may be collected for further analysis, e.g., downstream molecular assessment and/or profiling, such as genomics, transcriptomics, proteomics, metabolomics, etc. Any of the methods or platforms disclosed herein may be used as a tool that permits a user to train one or more models (e.g., from the modeling library) for cell clustering and/or cell classification. For example, a user may provide initial image dataset of a sample to the platform, and the platform may process the initial set of image data. Based on the processing, the platform may determine a number of labels and/or an amount of data that the user needs to train the one or more models, based on the initial image dataset of the sample. In some examples, the platform may determine that the initial set of image data may be insufficient to provide an accurate cell classification or cell morphology map. For example, the platform may plot an initial cell morphology map and recommend to the user the number of labels and/or the amount of data needed to for enhanced processing, classification, and/or sorting, based on proximity (or separability), correlation, or commonality of the datapoints in the map (e.g., whether there is no distinguishable clusters within the map, whether the clusters within the map are too close to each other, etc.). In another example, the platform may allow the user to select different model (e.g., clustering model) or classifier, different combinations of models or classifiers, to re-analyze the initial set of image data.

Any of the methods or platforms disclosed herein may be used to determine quality or state of the image(s) of the cell, that of the cell, or that of a sample comprising the cell. The quality or state of the cell may be determined at a single cell level. In another example, the quality or state of the cell may be determined at an aggregate level (e.g., as a whole sample, or as a portion of the sample). The quality or state may be determined and reported based on, e.g., a number system (e.g., a number scale from about 1 to about 10, a percentage scale from about 1% to about 100%), a symbolic system, or a color system. For example, the quality or state may be indicative of a preparation or priming condition of the sample (e.g., whether the sample has a sufficient number of cells, whether the sample has too much artifacts, debris, etc.) or indicative of a viability of the sample (e.g., whether the sample has an amount of “dead” cells above a predetermined threshold).

Any of the methods or platforms disclosed herein may be used to sort cells in silico (e.g., prior to actual sorting of the cells using a microfluidic channel). The in silico sorting may be, e.g., to discriminate among and/or between, e.g., multiple different cell types (e.g., different types of cancer cells, different types of immune cells, etc.), cell states, cell qualities. The methods and platforms disclosed herein may utilize pre-determined morphometric features (e.g., provided in the platform) for the discrimination. In another example, newly abstracted morphometric features may be abstracted (e.g., generated) based on the input data for the discrimination. In some examples, new model(s) and/or classifier(s) may be trained or generated to process the image data. In some examples, the newly abstracted morphometric features may be used to discriminate among and/or between, e.g., multiple different cell types, cell states, cell qualities that are known. In another example, the newly abstracted morphometric features may be used to create new class (or classifications) to sort the cells (e.g., in silico or via the microfluidic system). The newly abstracted morphometric features as disclosed herein may enhance accuracy or sensitivity of cell sorting (e.g., in silico or via the microfluidic system).

Subsequent to the in silico sorting of the cells, the actual cell sorting of the cells (e.g., via the microfluidic system or cartridge) based on the in silico sorting may be performed within less than about 1 hours, less than about 30 minutes, less than about 10 minutes, less than about 5 minutes, less than about 1 minute, less than about 30 seconds, less than about 10 seconds, less than about 5 seconds, less than about 1 second, or less. In some examples, the in silico sorting and the actual sorting may occur in real-time.

In any of the methods or platforms disclosed herein, the model(s) and/or classifier(s) may be validated (e.g., for the ability to demonstrate accurate cell classification performance). Non-limiting examples of validation metrics that may be utilized may include, but are not limited to, threshold metrics (e.g., accuracy, F-measure, Kappa, Macro-Average Accuracy, Mean-Class-Weighted Accuracy, Optimized Precision, Adjusted Geometric Mean, Balanced Accuracy, etc.), the ranking methods and metrics (e.g., receiver operating characteristics (ROC) analysis or “ROC area under the curve (ROC AUC)”), and the probabilistic metrics (e.g., root-mean-squared error). For example, the model(s) or classifier(s) may be determined to be balanced or accurate when the ROC AUC is greater than 0.5, greater than about 0.55, greater than about 0.6, greater than about 0.65, greater than about 0.7, greater than about 0.75, greater than about 0.8, greater than about 0.85, greater than about 0.9, greater than about 0.91, greater than about 0.92, greater than about 0.93, greater than about 0.94, greater than about 0.95, greater than about 0.96, greater than about 0.97, greater than about 0.98, greater than about 0.99, or more.

In any of the methods or platforms disclosed herein, the image(s) of the cell(s) may be obtained when the cell(s) are prepared and diluted in a sample (e.g., a buffer sample). The cell(s) may be diluted, e.g., in comparison to real-life concentrations of the cell in the tissue (e.g., solid tissue, blood, serum, spinal fluid, urine, etc.) to a dilution concentration. The methods or platforms disclosed herein may be compatible with a sample (e.g., a biological sample or derivative thereof) that is diluted by a factor of about 500 to about 1,000,000. The methods or platforms disclosed herein may be compatible with a sample that is diluted by a factor of at least about 500. The methods or platforms disclosed herein may be compatible with a sample that is diluted by a factor of at most about 1,000,000. The methods or platforms disclosed herein may be compatible with a sample that is diluted by a factor of about 500 to about 1,000, about 500 to about 10,000, about 500 to about 100,000, about 500 to about 1,000,000, about 1,000 to about 10,000, about 1,000 to about 1,000 to about 100,000, about 1,000 to about 1,000,000, about 2,000 to about 10,000, about 2,000 to about 100,000, about 2,000 to about 1,000,000, about 5,000 to about 10,000, about 5,000 to about 100,000, about 5,000 to about 1,000,000, about 10,000 to about 100,000, about 10,000 to about 1,000,000, about 20,000 to about 100,000, about 20,000 to about 1,000,000, about 50,000 to about 100,000, about 50,000 to about 1,000,000 about 100,000 to about 1,000,000, about 200,000 to about 1,000,000, or about 500,000 to about 1,000,000. The methods or platforms disclosed herein may be compatible with a sample that is diluted by a factor of at least about 500, e.g., at least about 1,000, at least about 2,000, at least about 5,000, at least about 10,000, at least about 20,000, at least about 50,000, at least about 100,000, at least about 200,000, at least about 500,000, or at least about 1,000,000 or more.

In any of the methods or platforms disclosed herein, the classifier may generate a prediction probability (e.g., based on the morphological clustering and analysis) that an individual cell or a cluster of cells belongs to a cell class (e.g., within a predetermined cell class provided in the CMA as disclosed herein), e.g., via a reporting module. The reporting module may communicate with the user via a GUI as disclosed herein. In another example, the classifier may generate a prediction vector that an individual cell or a cluster of cells belongs to a plurality of cell classes (e.g., a plurality of all of predetermined cell classes from the CMA as disclosed herein). The vector may be ID (e.g., a single row of different cell classes), 2D (e.g., two dimensions, such as tissue origin vs. cell type), 3D, etc. In some examples, based on processing and analysis of image data obtained from a sample, the classifier may generate a report showing a composition of the sample, e.g., a distribution of one or more cell types, each cell type indicated with a relative proportion within the sample. Each cell of the sample may also be annotated with a most probable cell type and one or more less probably cell types.

Any one of the methods and platforms disclosed herein may be capable of processing image data of one or more cells to generate one or more morphometric maps of the one or more cells. Non-limiting examples of morphometric models may be utilized to analyze one or more images of single cells (or cell clusters) may include, e.g., simple morphometries (e.g., based on lengths, widths, masses, angles, ratios, areas, etc.), landmark-based geometric morphometries (e.g., spatial information, intersections, etc. of one or more components of a cell), procrustes-based geometric morphometries (e.g., by removing non-shape information that is altered by translation, scaling, and/or rotation from the image data), Euclidean distance matrix analysis, diffeomorphometry, and outline analysis. The morphometric map(s) may be multi-dimensional (e.g., 2D, 3D, etc.). The morphometric map(s) may be reported to the user via the GUI.

Any of the methods or platforms disclosed herein (e.g., the analysis module) may be used to process, analyze, classify, and/or compare two or more samples (e.g., at least about 2, for example, at least about 3, at least about 4, at least about 5, at least about 6, at least about 7, at least about 8, at least about 9, at least about 10, or more test samples). The two or more samples may each be analyzed to determine a morphological profile (e.g., a cell morphology map) of each sample, and to compare the morphological profiles of the samples.

Any of the platforms disclosed herein (e.g., cell analysis platform) may provide an inline end-to-end pipeline solution for continuous labeling and/or sorting of multiple different cell types and/or states based at least in part on (e.g., based solely on) morphological analysis of imaging data provided. A modeling library used by the platform may be scalable for large amount of data, extensible (e.g., one or more models or classifiers modified), and/or generalizable (e.g., more resistant to data perturbations-such as artifacts, debris, random objects in the background, image/video distortions-between samples). Any of the modeling library may be removed or updated with new model automatically by the machine learning algorithms or artificial intelligence, or by the user.

Any of the methods and platforms disclosed herein may adjust one or more parameters of the microfluidic system as disclosed herein. As cells are flowing through a flow channel, an imaging module (e.g., sensors, cameras) may capture image(s)/video(s) of the cells and generate new image data. The image data may be processed and analyzed (e.g., in real-time) by the methods and platforms of the present disclosure to train a model (e.g., machine learning model) to determine whether or not one or more parameters of the microfluidic system.

In some examples, the model(s) may determine that the cells are flowing too fast or too slow, and send an instruction to the microfluidic system to adjust (i) the velocity of the cells (e.g., using adjusting velocity of the fluid medium carrying the cells) and/or (ii) image recording rate of a camera that is capturing images/videos of cells flowing through the flow channel.

In some examples, the model(s) may determine that the cells are in-focus or out-of-focus in the images/videos, and send an instruction to the microfluidic system to (i) adjust a positioning of the cells within the cartridge (e.g., move the cell towards or away from the center of the flow channel via, for example, hydrodynamic focusing and/or inertial focusing) and/or (ii) adjust a focal length/plane of the camera that is capturing images/videos of cells flowing through the flow channel. Adjusting the focal length/plane may be performed for the same cell that has been analyzed (e.g., adjusting focal length/plane of a camera that is downstream) or a subsequent cell. Adjusting the focal length/plane may enhance clarity or reduce blurriness in the images. The focal length/plane may be adjusted based on a classified type or state of the cell. In some examples, adjusting the focal length/plane may allow enhanced focusing/clarity on all parts of the cell. In some examples, adjusting the focal length/plane may allow enhanced focusing/clarity on different portions (but not all parts) of the cell. Without wishing to be bound by any particular theory, out-of-focus images may be usable for any of the methods disclosed herein to extract morphological feature(s) of the cell that otherwise may not be abstracted from in-focus images, or vice versa. Thus, in some examples, instructing the imaging module to capture both in-focus and out-of-focus images of the cells may enhance accuracy of any of the analysis of cells disclosed herein.

In another example, the model(s) may send an instruction to the microfluidic system to modify the flow and adjust an angle of the cell relative to the camera, to adjust focus on different portions of the cell or a subsequent cell. Different portions as disclosed herein may comprise an upper portion, a mid portion, a lower portion, membrane, nucleus, mitochondria, etc. of the cell.

In order to image cells at the right focus (with respect to height or z dimension), what is conventionally done is to calculate the “focus measure” of an image using information theoretic methods like Fourier Transform or Laplace transform.

In some examples, bi-directional out-of-focus (OOF) images cells (e.g., one or more first images that are OOF in a first direction, and one or more second images that are OOF in as second direction that is different—such as opposite—from the first direction). For example, images that are OOF in two opposite directions may be called “bright OOF” image(s) and “dark OOF” image(s), which may be obtained by changing the z-focus bi-directionally. A classifier as disclosed herein may be trained with a image data comprising both bright OOF image(s) and dark OOF image(s). The trained classifiers may be used to run inferences (e.g., in real-time) on new image data of cells to classify each image as bright OOF image, dark OOF image, and optionally image that is not OOF (e.g., not OOF relative to the bright/dark OOF images). The classifier may also measure a percentage of bright OOF image, a percentage of dark OOF image, or a percentage of both bright and dark OOF images within the image data. For example, if any of the percentage of bright OOF image, the percentage of dark OOF image, or the percentage of both bright and dark OOF images is above a threshold value (e.g., a predetermined threshold value), then the classifier may determine that the imaging device (e.g., by the microfluidic system as disclosed herein) may not be imaging cells at the right focal length/plane. The classifier may instruct the user, via GUI of a user device, to adjust the imaging device's focal length/plane. In some examples, the classifier may determine, based on analysis of the image data comprising OOF images, direction and degree of adjustment of focal length/plane that may be required to adjust the imaging device, to yield a reduced amount of OOF imaging. In some examples, the classifier and the microfluidic device may be operatively coupled to a controller, such that the focal length/plane of the imaging device may be adjusted automatically upon determination of the classifier.

A threshold (e.g., a predetermined threshold) of a percentage of OOF images (e.g., bright OOF, dark OOF, or both) may be about 0.1% to about 20%. A threshold (e.g., a predetermined threshold) of a percentage of OOF images (e.g., bright OOF, dark OOF, or both) may be at least about 0.1%. A threshold (e.g., a predetermined threshold) of a percentage of OOF images (e.g., bright OOF, dark OOF, or both) may be at most about 20%. A threshold (e.g., a predetermined threshold) of a percentage of OOF images (e.g., bright OOF, dark OOF, or both) may be about 0.1% to about 1%, about 0.1% to about 10%, about 0.5% to about 1%, about 0.5% to about 10%, about 1% to about 10%, about 2% to about 10%, about 4% to about 10%, about 6% to about 10%, about 8% to about 10, about 10% to about 15%, about 10% to about 20%, or about 15% to about 20%. A threshold (e.g., a predetermined threshold) of a percentage of OOF images (e.g., bright OOF, dark OOF, or both) may be at least about 0.1%—e.g., at least about 0.5%, or at least about 1%, or at least about 2%, or at least about 4%, or at least about 6%, or at least about 8%, or at least about 10%, or at least about 15%, or at least about 20%, or higher.

In some examples, the model(s) may determine that images of different modalities are needed for any of the analysis disclosed herein. Images of varying modalities may comprise a bright field image, a dark field image, a fluorescent image (e.g., of cells stained with a dye), an in-focus image, an out-of-focus image, a greyscale image, a monochrome image, a multi-chrome image, etc.

Any of the models or classifiers disclosed herein may be trained on a set of image data that is annotated with one imaging modality. In another example, the models/classifiers may be trained on set of image data that is annotated with a plurality of different imaging modalities (e.g., about 2, about 3, about 4, about 5, or more different imaging modalities). Any of the models/classifiers disclosed herein may be trained on a set of image data that is annotated with a spatial coordinate indicative of a position or location within the flow channel. Any of the models/classifiers disclosed herein may be trained on a set of image data that is annotated with a timestamp, such that a set of images may be processed based on the time they are taken.

An image of the image data may be processed in various image processing methods, such as horizontal or vertical image flips, orthogonal rotation, gaussian noise, contrast variation, or noise introduction to mimic microscopic particles or pixel-level aberrations. One or more of the processing methods may be used to generate replicas of the image or analyze the image. In some examples, the image may be processed into a lower-resolution image or a lower-dimension image (e.g., by using one or more deconvolution algorithm).

In any of the methods disclosed herein, processing an image or video from image data may comprise identifying, accounting for, and/or excluding one or more artifacts from the image/video, either automatically or manually by a user. Upon identification, the artifact(s) may be fed into any of the models or classifiers, to train image processing or image analysis. The artifact(s) may be accounted for when classifying the type or state of one or more cells in the image/video. The artifact(s) may be excluded from any determination of the type or state of the cell(s) in the image/video. The artifact(s) may be removed in silico by any of the models/classifiers disclosed herein, and any new replica or modified variant of the image/video excluding the artifact(s) may be stored in a database as disclosed herein. The artifact(s) may be, for example, from debris (e.g., dead cells, dust, etc.), optical conditions during capturing the image/video of the cells (e.g., lighting variability, over-saturation, under-exposure, degradation of the light source, etc.), external factors (e.g., vibrations, misalignment of the microfluidic chip relative to the lighting or optical sensor/camera, power surges/fluctuations, etc.), and changes to the microfluidic system (e.g., deformation/shrinkage/expansion of the microfluidic channel or the microfluidic chip as a whole). The artifacts may be known. The artifacts may be unknown, and the models or classifiers disclosed herein may be configured to define one or more parameters of a new artifact, such that the new artifact may be identified, accounted for, and/or excluded in image processing and analysis.

In some examples, a plurality of artifacts disclosed herein may be identified, accounted for, and/or excluded during image/video processing or analysis. The plurality of artifacts may be weighted the same (e.g., determined to have the same degree of influence on the image/video processing or analysis) or may have different weights (e.g., determined to have different degrees of influence on the image/video processing or analysis). Weight assignments to the plurality of artifacts may be instructed manually by the user or determined automatically by the models/classifiers disclosed herein.

In some examples, one or more reference images or videos of the flow channel (e.g., with or without any cell) may be stored in a database and used as a frame of reference to help identify, account for, and/or exclude any artifact. The reference image(s)/video(s) may be obtained before use of the microfluidics system. The reference image(s)/video(s) may be obtained during the use of the microfluidics system. The reference image(s)/video(s) may be obtained periodically during the use of the microfluidics system, such as, each time the optical sensor/camera captures at least about 10—e.g., at least about 100, at least about 1,000, at least about 10,000, or at least about 100,000, or higher, images. The reference image(s)/video(s) may be obtained periodically during the use of the microfluidics system, such as, each time the microfluidics system passes at least about 10—e.g., at least about 100, at least about 1,000, at least about 10,000, or at least about 100,000, or higher, cells. The reference image(s)/video(s) may be obtained at landmark periods during the use of the microfluidics system, such as, when the optical sensor/camera captures at least about 10,—e.g., at least about 100, at least about 1,000, at least about 10,000, or at least about 100,000 images, or higher. The reference image(s)/video(s) may be obtained at landmark periods during the use of the microfluidics system, such as, when the microfluidics system passes at least about 10,—e.g., at least about 100, at least about 1,000, at least about 10,000, or at least about 100,000 images or higher.

The method and the platform as disclosed herein may be utilized to process (e.g., modify, analyze, classify) the image data at a rate of about 1,000 images/second to about 100,000,000 images/second. The rate of image data processing may be at least about 1,000 images/second. For example, the rate of image data processing may be at most about 100,000,000 images/second. For example, the he rate of image data processing may be about 1,000 images/second to about 10,000 images/second, about 1,000 images/second to about 100,000 images/second, about 1,000 images/second to about 1,000,000 images/second, about 1,000 images/second to about 10,000,000 images/second, about 1,000 images/second to about 100,000,000 images/second, about 10,000 images/second to about 100,000 images/second, about 10,000 images/second to about 1,000,000 images/second, about 10,000 images/second to about 10,000,000 images/second, about 10,000 images/second to about 100,000,000 images/second, about 100,000 images/second to about 1,000,000 images/second, about 100,000 images/second to about 10,000,000 images/second, about 100,000 images/second to about 100,000,000 images/second, about 1,000,000 images/second to about 10,000,000 images/second, about 1,000,000 images/second to about 100,000,000 images/second, or about 10,000,000 images/second to about 100,000,000 images/second or higher. In some examples, the rate of image data processing may be about 1,000 images/second, images/second,—e.g., about 10,000 images/second, about 100,000 images/second, about 1,000,000 images/second, about 10,000,000 images/second, or about 100,000,000 images/second or higher.

The method and the platform as disclosed herein may be utilized to process (e.g., modify, analyze, classify) the image data at a rate of about 1,000 cells/second to about 100,000,000 cells/second. The rate of image data processing may be at least about 1.000 cells/second. For example, the rate of image data processing may be at most about 100,000,000 cells/second. For example, the rate of image data processing may be about 1,000 cells/second to about 10,000 cells/second, about 1,000 cells/second to about 100,000 cells/second, about 1,000 cells/second to about 1,000,000 cells/second, about 1,000 cells/second to about 10,000,000 cells/second, about 1,000 cells/second to about 100,000,000 cells/second, about 10,000 cells/second to about 100,000 cells/second, about 10,000 cells/second to about 1,000,000 cells/second, about 10,000 cells/second to about 10,000,000 cells/second, about 10,000 cells/second to about 100,000,000 cells/second, about 100,000 cells/second to about 10,000,000 cells/second, about 100,000 cells/second to about 100,000,000 cells/second, about 1,000,000 cells/second to about 10,000,000 cells/second, about 1,000,000 cells/second to about 100,000,000 cells/second, or about 10,000,000 cells/second to about 100,000,000 cells/second or higher. In some examples, the rate of image data processing may be about 1,000 cells/second,—e.g., about 5,000 cells/second, about 10,000 cells/second, about 50,000 cells/second, about 100,000 cells/second, about 500,000 cells/second, about 1,000,000 cells/second, about 5,000,000 cells/second, about 10,000,000 cells/second, about 50,000,000 cells/second, or about 100,000,000 cells/second or higher.

The method and the platform as disclosed herein may be utilized to process (e.g., modify, analyze, classify) the image data at a rate of about 1,000 datapoints/second to about 100,000,000 datapoints/second. For example, the rate of image data processing may be at least about 1,000 datapoints/second. For example, the rate of image data processing may be at most about 100,000,000 datapoints/second. For example, the rate of image data processing may be about 1,000 datapoints/second to about 10,000 datapoints/second, about 1,000 datapoints/second to about 100,000 datapoints/second, about 1,000 datapoints/second to about 1,000,000 datapoints/second, about 1,000 datapoints/second to about 10,000,000 datapoints/second, about 1,000 datapoints/second to about 100,000,000 datapoints/second, about 10,000 datapoints/second to about 100,000 datapoints/second, about 10,000 datapoints/second to about 1,000,000 datapoints/second, about 10,000 datapoints/second to about 10,000,000 datapoints/second, about 10,000 datapoints/second to about 100,000,000 datapoints/second, about 100,000 datapoints/second to about 1,000,000 datapoints/second, about 100,000 datapoints/second to about 10,000,000 datapoints/second, about 100,000 datapoints/second to about 100,000,000 datapoints/second, about 1,000,000 datapoints/second to about 10,000,000 datapoints/second, about 1,000,000 datapoints/second to about 100,000,000 datapoints/second, or about 10,000,000 datapoints/second to about 100,000,000 datapoints/second or higher. In some examples, the rate of image data processing may be about 1,000 datapoints/second,—e.g., about 5,000 datapoints/second, about 10,000 datapoints/second, about 50,000 datapoints/second, about 100,000 datapoints/second, about 500,000 datapoints/second, about 1,000,000 datapoints/second, about 5,000,000 datapoints/second, about 10,000,000 datapoints/second, about 50,000,000 datapoints/second, or about 100,000,000 datapoints/second or higher.

Any of the methods or platforms disclosed herein may be operatively coupled to an online crowdsourcing platform. The online crowdsourcing platform may comprise any of the database disclosed herein. For example, the database may store a plurality of single cell images that are grouped into morphologically-distinct clusters corresponding to a plurality of cell classes (e.g., predetermined cell types or states). The online crowdsourcing platform may comprise one or more models or classifiers as disclosed herein (e.g., a modeling library comprising one or more machine learning models/classifiers as disclosed herein). The online crowdsourcing platform may comprise a web portal for a community of users to share contents, e.g., (1) upload, download, search, curate, annotate, or edit one or more existing images or new images into the database, (2) train or validate the one or more model(s)/classifier(s) using datasets from the database, and/or (3) upload new models into the modeling library. In some examples, the online crowdsourcing platform may allow users to buy, sell, share, or exchange the model(s)/classifier(s) with one another.

In some examples, the web portal may be configured to generate incentives for the users to update the database with new annotated cell images, model(s), and/or classifier(s). Incentives may be monetary. Incentives may be additional access to the global CMA, model(s), and/or classified s). In some examples, the web portal may be configured to generate incentives for the users to download, use, and review (e.g., rate or leave comments) any of the annotated cell images, model(s), and/or classifier(s) from, e.g., other users.

In some examples, a global cell morphology atlas (global CMA) may be generated using collecting (i) annotated cell images, (ii) cell morphology maps or ontologies, (iii), and/or (iv) classifiers from the users using the web portal. The global CMA may then be shared with the users via the web portal. All users may have access to the global CMA. In another example, specifically defined users may have access to specifically defined portions of the global CMA. For example, cancer centers may have access to “cancer cells” portion of the global CMA, e.g., using a subscription based service. In a similar fashion, global models or classifiers may be generated based on the annotated cell images, model(s), and/or classifiers that are collected from the users using the web portal.

### Microfluidic Systems and Methods Thereof

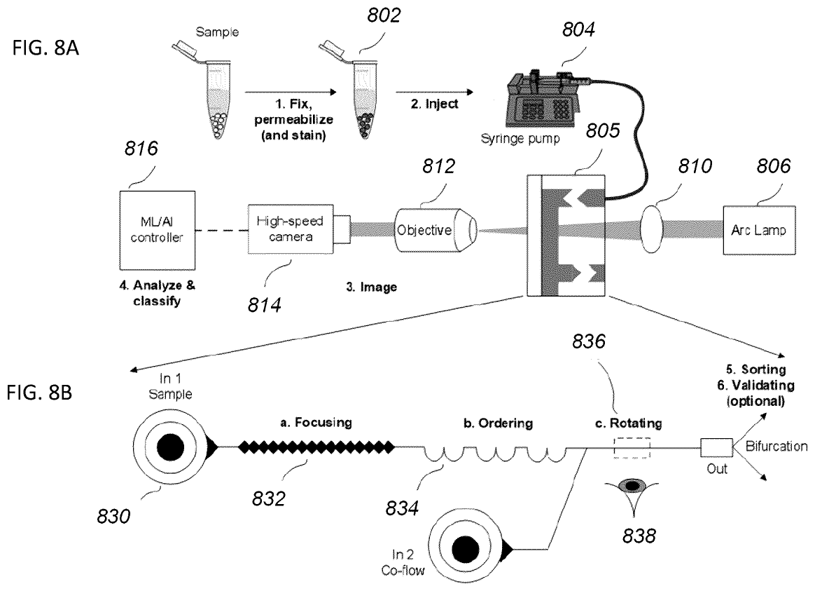

FIG. 8A shows a schematic illustration of the cell sorting system, as disclosed herein, with a cartridge design (e.g., a microfluidic design), with further details illustrated in FIG. 8B. The cell sorting system may be operatively coupled to a controller running the human foundation model 180 and data suite 190 in a manner such as described elsewhere herein. Such controller may be configured to perform any of the methods disclosed herein. Such controller may be operatively coupled to, or included within, any of the platforms disclosed herein. In operation, a sample 802 is prepared and injected by a pump 804 (e.g., a syringe pump) into a cartridge 805 (corresponding to cartridge 110 described with reference to FIG. 1 or cartridge 210 described with reference to FIG. 2) or flow-through device. In some examples, the cartridge 805 is a microfluidic device. Although FIG. 8A illustrates a classification and/or sorting system utilizing a syringe pump, any of a number of perfusion systems may be used such as (but not limited to) gravity feeds, peristalsis, or any of a number of pressure systems. In some examples, the sample is prepared by fixation and staining. In some examples, the sample comprises live cells. As may readily be appreciated, the specific manner in which the sample is prepared is largely dependent upon the requirements of a specific application.

Examples of the pump or other suitable flow unit may be, but are not limited to, a syringe pump, a vacuum pump, an actuator (e.g., linear, pneumatic, hydraulic, etc.), a compressor, or any other suitable device to exert pressure (positive, negative, alternating thereof, etc.) to a fluid that may or may not comprise one or more particles (e.g., one or more cells to be classified, sorted, and/or analyzed). The pump or other suitable flow unit may be configured to raise, compress, move, and/or transfer fluid into or away from the microfluidic channel. In some examples, the pump or other suitable flow unit may be configured to deliver positive pressure, alternating positive pressure and vacuum pressure, negative pressure, alternating negative pressure and vacuum pressure, and/or only vacuum pressure. The cartridge of the present disclosure may comprise (or otherwise be in operable communication with) at least about 1, e.g., at least about 2, at least about 3, at least about 4, at least about 5, at least about 6, at least about 7, at least about 8, at least about 9, at least about 10, or more, pumps or other flow units. The cartridge may comprise at most about 10,—e.g., at most about 9, at most about 8, at most about 7, at most about 6, at most about 5, at most about 4, at most about 3, at most about 2, or at most about 1 pumps or other suitable flow units.

Each pump or other suitable flow unit may be in fluid communication with at least about 1, e.g., at least about 2, at least about 3, at least about 4, at least about 5, at least about 6, at least about 7, at least about 8, at least about 9, at least about 10, or more sources of fluid. Each flow unit may be in fluid communication with at most about 10, e.g., at most about 9, at most about 8, at most about 7, at most about 6, at most about 5, at most about 4, at most about 3, at most about 2, or at most about 1 fluid. The fluid may contain the particles (e.g., cells). In another example, the fluid may be particle-free. The pump or other suitable flow unit may be configured to maintain, increase, and/or decrease a flow velocity of the fluid within the microfluidic channel of the flow unit. Thus, the pump or other suitable flow unit may be configured to maintain, increase, and/or decrease a flow velocity (e.g., downstream of the microfluidic channel) of the particles. The pump or other suitable flow unit may be configured to accelerate or decelerate a flow velocity of the fluid within the microfluidic channel of the flow unit, thereby accelerating or decelerating a flow velocity of the particles.

The fluid may be liquid or gas (e.g., air, argon, nitrogen, etc.). The liquid may be an aqueous solution (e.g., water, buffer, saline, etc.). In another example, the liquid may be oil. In some examples, only one or more aqueous solutions may be directed through the microfluidic channels. In another example, only one or more oils may be directed through the microfluidic channels. In another alternative, both aqueous solution(s) and oil(s) may be directed through the microfluidic channels. In some examples, (i) the aqueous solution may form droplets (e.g., emulsions containing the particles) that are suspended in the oil, or (ii) the oil may form droplets (e.g., emulsions containing the particles) that are suspended in the aqueous solution. As may readily be appreciated, any perfusion system, including but not limited to peristalsis systems and gravity feeds, appropriate to a given classification and/or sorting system may be utilized.

As noted above, the cartridge 805 may be implemented as a fluidic device that focuses cells from the sample into a single streamline that is imaged continuously. In the illustrated example, the cell line is illuminated by a light source 806 (e.g., a lamp, such as an arc lamp) and an optical system 810 that directs light onto an imaging region 838 of the cartridge 805. An objective lens system 812 magnifies the cells by directing light toward the sensor of a high-speed camera system 814.

In some examples, a 10×, 20×, 40×, 60×, 80×, 100×, or 200× objective is used to magnify the cells. In some examples, a 10×, objective is used to magnify the cells. In some examples, a 20× objective is used to magnify the cells. In some examples, a 40× objective is used to magnify the cells. In some examples, a 60× objective is used to magnify the cells. In some examples, a 80× objective is used to magnify the cells. In some examples, a 100× objective is used to magnify the cells. In some examples, a 200× objective is used to magnify the cells. In some examples, a 10× to a 200× objective is used to magnify the cells, for example a 10×-20×, a 10×-40×, a 10×-60×, a 10×-80×, or 10×-100× objective is used to magnify the cells. As may readily be appreciated by a person having ordinary skill in the art, the specific magnification utilized may vary greatly and is largely dependent upon the requirements of a given imaging system and cell types of interest.

In some examples, one or more imaging devices may be used to capture images of the cell. In some examples, the imaging device is a high-speed camera. In some examples, the imaging device is a high-speed camera with a micro-second exposure time. In some instances, the exposure time is about 1 millisecond. In some instances, the exposure time is between about 1 millisecond (ms) and about 0.75 millisecond. In some instances, the exposure time is between about 1 ms and about 0.50 ms. In some instances, the exposure time is between about 1 ms and about 0.25 ms. In some instances, the exposure time is between about 0.75 ms and about 0.50 ms. In some instances, the exposure time is between about 0.75 ms and about 0.25 ms. In some instances, the exposure time is between about 0.50 ms and about 0.25 ms. In some instances, the exposure time is between about 0.25 ms and about 0.1 ms. In some instances, the exposure time is between about 0.1 ms and about 0.01 ms. In some instances, the exposure time is between about 0.1 ms and about 0.001 ms. In some instances, the exposure time is between about 0.1 ms and about 1 microsecond (ps). In some examples, the exposure time is between about 1 ps and about 0.1 ps. In some examples, the exposure time is between about 1 ps and about 0.01 ps. In some examples, the exposure time is between about 0.1 ps and about 0.01 ps. In some examples, the exposure time is between about 1 ps and about 0.001 ps. In some examples, the exposure time is between about 0.1 ps and about 0.001 ps. In some examples, the exposure time is between about 0.01 ps and about 0.001 ps.

In some examples, the cartridge 805 may comprise at least about 1, e.g., at least about 2, at least about 3, at least about 4, at least about 5, at least about 6, at least about 7, at least about 8, at least about 9, at least about 10, or more, imaging devices (e.g., the high-speed camera system 814) on or adjacent to the imaging region 838. In some examples, the cartridge may include at most about 10, e.g., at most about 9, at most about 8, at most about 7, at most about 6, at most about 5, at most about 4, at most about 3, at most about 2, or at most about 1 imaging device on or adjacent to the imaging region 838. In some examples, the cartridge 805 may comprise a plurality of imaging devices. Each of the plurality of imaging devices may use light from a same light source. In another example, each of the plurality of imaging devices may use light from different light sources. The plurality of imaging devices may be configured in parallel and/or in series with respect to one another. The plurality of imaging devices may be configured on one or more sides (e.g., two adjacent sides or two opposite sides) of the cartridge 805. The plurality of imaging devices may be configured to view the imaging region 838 along a same axis or different axes with respect to (i) a length of the cartridge 805 (e.g., a length of a straight channel of the cartridge 805) or (ii) a direction of migration of one or more particles (e.g., one or more cells) in the cartridge 805.

One or more imaging devices of the present disclosure may be stationary while imaging one or more cells, e.g., at the imaging region 838. In another example, one or more imaging devices may move with respect to the flow channel (e.g., along the length of the flow channel, towards and/or away from the flow channel, tangentially about the circumference of the flow channel, etc.) while imaging the one or more cells. In some examples, the one or more imaging devices may be operatively coupled to one or more actuators, such as, for example, a stepper actuator, linear actuator, hydraulic actuator, pneumatic actuator, electric actuator, magnetic actuator, and mechanical actuator (e.g., rack and pinion, chains, etc.).

In some examples, the cartridge 805 may comprise at least about 1, e.g., at least about 2, about 3, about 4, about 5, about 6, about 7, about 8, about 9, about 10, or more, imaging regions (e.g., the imaging region 838). In some examples, the cartridge 805 may comprise at most about 10—e.g., at most about 9, about 8, about 7, about 6, about 5, about 4, about 3, about 2, or about 1 imaging region. In some examples, the cartridge 815 may comprise a plurality of imaging regions, and the plurality of imaging regions may be configured in parallel and/or in series with respect to each another. The plurality of imaging regions may or may not be in fluid communication with each other. In an example, a first imaging region and a second imaging region may be configured in parallel, such that a first fluid that passes through the first imaging region does not pass through a second imaging region. In another example, a first imaging region and a second imaging region may be configured in series, such that a first fluid that passes through the first imaging region also passes through the second imaging region.

The imaging device(s) (e.g., the high-speed camera) of the imaging system may comprise an electromagnetic radiation sensor (e.g., IR sensor, color sensor, etc.) that detects at least a portion of the electromagnetic radiation that is reflected by and/or transmitted from the cartridge or any content (e.g., the cell) in the cartridge. The imaging device may be in operative communication with one or more sources (e.g., at least about 1, e.g., about 2, about 3, about 4, about 5, or more) of the electromagnetic radiation. The electromagnetic radiation may comprise one or more wavelengths from the electromagnetic spectrum including, but not limited to x-rays (about 0.1 nanometers (nm) to about 10.0 nm; or about 10¹⁸ Hertz (Hz) to about 10¹⁶ Hz), ultraviolet (UV) rays (about 10.0 nm to about 380 nm; or about 8×10¹⁶ Hz to about 10¹⁵ Hz), visible light (about 380 nm to about 750 nm; or about 8×10¹⁴ Hz to about 4×10¹⁴ Hz), infrared (IR) light (about 750 nm to about 0.1 centimeters (cm); or about 4×10¹⁴ Hz to about 5×10¹¹ Hz), and microwaves (about 0.1 cm to about 100 cm; or about 108 Hz to about 5×10¹¹ Hz). In some examples, the source(s) of the electromagnetic radiation may be ambient light, and thus the cell sorting system may not have an additional source of the electromagnetic radiation.

The imaging device(s) may be configured to take a two-dimensional image (e.g., one or more pixels) of the cell and/or a three-dimensional image (e.g., one or more voxels) of the cell.

As may readily be appreciated, the exposure times may differ across different systems and may largely be dependent upon the requirements of a given application or the limitations of a given system such as but not limited to flow rates. Images are acquired and may be analyzed using an image analysis algorithm.

In some examples, the images are acquired and analyzed post-capture. In some examples, the images are acquired and analyzed in real-time continuously. Using object tracking software, single cells may be detected and tracked while in the field of view of the camera.

Background subtraction may then be performed. In a number of examples, the cartridge 806 causes the cells to rotate as they are imaged, and multiple images of each cell are provided to a computing system 816 for analysis. In some examples, the multiple images comprise images from a plurality of cell angles.

The flow rate and channel dimensions may be determined to obtain multiple images of the same cell from a plurality of different angles (i.e., a plurality of cell angles). A degree of rotation between an angle to the next angle may be uniform or non-uniform. In some examples, a full 360° view of the cell is captured. In some examples, 4 images are provided in which the cell rotates 90° between successive frames. In some examples, 8 images are provided in which the cell rotates 45° between successive frames. In some examples, 24 images are provided in which the cell rotates 15° between successive frames. In some examples, at least three or more images are provided in which the cell rotates at a first angle between a first frame and a second frame, and the cell rotates at a second angle between the second frame and a third frame, wherein the first and second angles are different. In some examples, less than the full 360° view of the cell may be captured, and a resulting plurality of images of the same cell may be sufficient to classify the cell (e.g., determine a specific type of the cell).

The cell may have a plurality of sides. The plurality of sides of the cell may be defined with respect to a direction of the transport (flow) of the cell through the channel. In some examples, the cell may comprise a stop side, a bottom side that is opposite the top side, a front side (e.g., the side towards the direction of the flow of the cell), a rear side opposite the front side, a left side, and/or a right side opposite the left side. In some examples, the image of the cell may comprise a plurality of images captured from the plurality of angles, wherein the plurality of images comprise: (1) an image captured from the top side of the cell, (2) an image captured from the bottom side of the cell, (3) an image captured from the front side of the cell, (4) an image captured from the rear side of the cell, (5) an image captured from the left side of the cell, and/or (6) an image captured from the right side of the cell.

In some examples, a two-dimensional “hologram” of a cell may be generated using superimposing the multiple images of the individual cell. The “hologram” may be analyzed to automatically classify characteristics of the cell based upon features including but not limited to the morphological features of the cell.

In some examples, at least about 1, e.g., at least about 2, at least about 3, at least about 4, at least about 5, at least about 6, at least about 7, at least about 8, at least about 9, or at least about 10, or more, images are captured for each cell. For example, in some examples, about 5 or more images are captured for each cell. For example, in some examples, from about 5 to about 10 images are captured for each cell. In some examples, 10 or more images are captured for each cell. In some examples, from about 10 to about 20 images are captured for each cell. In some examples, about 20 or more images are captured for each cell. In some examples, from about 20 to about 50 images are captured for each cell. In some examples, about 50 or more images are captured for each cell. In some examples, from about 50 to about 100 images are captured for each cell. In some examples, at least about 1, e.g., at least about 2, at least about 3, at least about 4, at least about 5, at least about 6, at least about 7, at least about 8, at least about 9, at least about 10, at least about 15, at least about 20, at least about 30, at least about 40, at least about 50, or more images may be captured for each cell at a plurality of different angles. In some examples, at most 50, e.g., at most about 40, at most about 30, at most about 20, at most about 15, at most about 10, at most about 9, at most about 8, at most about 7, at most about 6, at most about 5, at most about 4, at most about 3, or at most about 2 images, or about one image, may be captured for each cell at a plurality of different angles.

In some examples, the imaging device is moved so as to capture multiple images of the cell from a plurality of angles. In some examples, the images are captured at an angle between 0 and 90 degrees to the horizontal axis. In some examples, the images are captured at an angle between 90 and 180 degrees to the horizontal axis. In some examples, the images are captured at an angle between 180 and 270 degrees to the horizontal axis. In some examples, the images are captured at an angle between 270 and 360 degrees to the horizontal axis. In some examples, multiple imaging devices (for, e.g., multiple cameras) are used wherein each device captures an image of the cell from a specific cell angle. In some examples, at least about 2, for example, at least about 3, at least about 4, at least about 5, at least about 6, at least about 7, at least about 8, at least about 9, or at least about 10 cameras, or more, are used. In some examples, more than about 10 cameras are used, wherein each camera images the cell from a specific cell angle.

As may readily be appreciated, the number of images that are captured is dependent upon the requirements of a given application or the limitations of a given system. In several examples, the cartridge has different regions to focus, order, and/or rotate cells. Although the focusing regions, ordering regions, and cell rotating regions are discussed as affecting the sample in a specific sequence, a person having ordinary skill in the art would appreciate that the various regions may be arranged differently, where the focusing, ordering, and/or rotating of the cells in the sample may be performed in any order. Regions within a microfluidic device implemented in accordance with an example of the disclosure are illustrated in FIG. 8B. Cartridge 805 may include a filtration region 830 to prevent channel clogging by aggregates/debris or dust particles. Cells pass through a focusing region 832 that focuses the cells into a single streamline of cells that are then spaced by an ordering region 834. In some examples, the focusing region utilizes “inertial focusing” to form the single streamline of cells. In some examples, the focusing region utilizes “hydrodynamic focusing” to focus the cells into the single streamline of cells. Optionally, prior to imaging, rotation may be imparted upon the cells by a rotation region 836. The optionally spinning cells may then pass through an imaging region 838 in which the cells are illuminated for imaging prior to exiting the cartridge. These various regions are described and discussed in further detail below. In some examples, the rotation region 836 may precede the imaging region 838. In some examples, the rotation region 836 may be a part (e.g., a beginning portion, a middle portion, and/or an end portion with respect to a migration of a cell within the cartridge) of the imaging region 838. In some examples, the imaging region 838 may be a part of the rotation region 836.

In some examples, a single cell is imaged in a field of view of the imaging device, e.g., camera. In some examples, multiple cells are imaged in the same field of view of the imaging device. In some examples, at least about 1, for example, at least about 2, at least about 3, at least about 4, at least about 5, at least about 6, at least about 7, at least about 8, at least about 9, or at least about 10 cells, or more, are imaged in the same field of view of the imaging device. In some examples, up to about 100 cells are imaged in the same field of view of the imaging device. For example, in some examples, about 10 to about 100 cells are imaged in the field of view, for example, about 10 to 20 cells, about 10 to about 30 cells, about 10 to about 40 cells, about 10 to about 50 cells, about 10 to about 60 cells, about 10 to about 80 cells, about 10 to about 90 cells, about 20 to about 30 cells, about 20 to about 40 cells, about 20 to about 50 cells, about 20 to about 60 cells, about 20 to about 70 cells, about 20 to about 80 cells, about 20 to about 90 cells, about 30 to about 40 cells, about 40 to about 50 cells, about 40 to about 60 cells, about 40 to about 70 cells, about 40 to about 80 cells, about 40 to about 90 cells, about 50 to about 60 cells, about 50 to about 70 cells, about 50 to about 80 cells, about 50 to about 90 cells, about 60 to about 70 cells, about 60 to about 80 cells, about 60 to about 90 cells, about 70 to about 80 cells, about 70 to about 90 cells, or about 90 to about 100 cells, or more, are imaged in the same field of view of the imaging device.

In some examples, only a single cell may be allowed to be transported across a cross-section of the flow channel perpendicular to the axis of the flow channel, corresponding to imaging area 114 described with reference to FIGS. 1-2. In some examples, a plurality of cells (e.g., at least about 2, for example about 3, about 4, about 5, or more cells; or at most about 5, for example, about 4, about 3, about 2, or about 1 cell) may be allowed to be transported simultaneously across the cross-section of the flow channel perpendicular to the axis of the flow channel. In such example, the imaging device (or the processor operatively linked to the imaging device) may be configured to track each of the plurality of cells as they are transported along the flow channel.

The imaging system may include, among other things, a camera, an objective lens system and a light source. In a number of examples, cartridges similar to those described above may be fabricated using standard 2D microfluidic fabrication techniques, requiring minimal fabrication time and cost.

Although specific classification and/or sorting systems, cartridges, and microfluidic devices are described above with respect to FIGS. 8A-8B, classification and/or sorting systems may be implemented in any of a variety of ways appropriate to the requirements of specific applications in accordance with various examples of the disclosure. Specific elements of microfluidic devices that may be utilized in classification and/or sorting systems in accordance with some examples of the disclosure are discussed further below.

In some examples, examples, the microfluidic system may comprise a microfluidic chip (e.g., comprising one or more microfluidic channels for flowing cells) operatively coupled to an imaging device (e.g., one or more cameras). A microfluidic device may comprise the imaging device, and the chip may be inserted into the device, to align the imaging device to an imaging region of a channel of the chip. To align the chip to the precise location for the imaging, the chip may comprise one or more positioning identifiers (e.g., pattern(s), such as numbers, letters, symbols, or other drawings) that may be imaged to determine the positioning of the chip (and thus the imaging region of the channel of the chip) relative to the device as a whole or relative to the imaging device. For image-based alignment (e.g., auto-alignment) of the chip within the device, one or more images of the chip may be capture upon its coupling to the device, and the image(s) may be analyzed by any of the methods disclosed herein (e.g., using any model or classifier disclosed herein) to determine a degree or score of chip alignment. The positioning identifier(s) may be a “guide” to navigate the stage holding the chip within the device to move within the device towards a correct position relative to the imaging unit. In some examples, rule-based image processing may be used to navigate the stage to a precise range of location or a precise location relative to the image unit. In some examples, machine learning/artificial intelligence methods as disclosed herein may be modified or trained to identify the pattern on the chip and navigate the stage to the precise imaging location for the image unit, to increase resilience.

In some examples, machine learning/artificial intelligence methods as disclosed herein may be modified or trained to implement reinforcement learning based alignment and focusing. The alignment process for the chip to the instrument or the image unit may involve moving the stage holding the chip in, e.g., either X or Y axis and/or moving the imaging plane on the Z axis. In the training process, (i) the chip may start at a X, Y, and Z position (e.g., randomly selected), (ii) based on one or more image(s) of the chip and/or the stage holding the chip, a model may determine a movement vector for the stage and a movement for the imaging plane, (iii) depending on whether such movement vector may take the chip closer to the optimum X, Y, and Z position relative to the image unit, an error term may be determined as a loss for the model, and (iv) the magnitude of the error may be either constant or be proportional to how far the current X, Y, and Z position is from an optimal X, Y, and Z position (e.g., may be predetermined). Such trained model may be used to determine, for example, the movement vector and/or movement of the movement for the imaging plane, to enhance relative alignment between the chip and the image unit (e.g., one or more sensors). The alignment may occur subsequent to capturing of the image(s). In another example, the alignment may occur real-time while capturing images/videos of the positioning identifier(s) of the chip.

One or more flow channels of the cartridge of the present disclosure may have various shapes and sizes. For example, referring to FIGS. 8A-8B, at least a portion of the flow channel (e.g., the focusing region 832, the ordering region 834, the rotation region 836, the imaging region 838, connecting region therebetween, etc.) may have a cross-section that is circular, triangular, square, rectangular, pentagonal, hexagonal, or any partial shape or combination of shapes thereof.

In some examples, the system of the present disclosure comprises straight channels with rectangular or square cross-sections. In some examples, the system of the present disclosure comprises straight channels with round cross-sections. In some examples, the system comprises straight channels with half-ellipsoid cross-sections. In some examples, the system comprises spiral channels. In some examples, the system comprises round channels with rectangular cross-sections. In some examples, the system comprises round channels with rectangular channels with round cross-sections. In some examples, the system comprises round channels with half-ellipsoid cross-sections. In some examples, the system comprises channels that are expanding and contracting in width with rectangular cross-sections. In some examples, the system comprises channels that are expanding and contracting in width with round cross-sections. In some examples, the system comprises channels that are expanding and contracting in width with half-ellipsoid cross-sections.

The flow channel may comprise one or more walls that are formed to focus one or more cells into a streamline. The flow channel may comprise a focusing region comprising the wall(s) to focus the cell(s) into the streamline. Focusing regions on a microfluidic device may take a disorderly stream of cells and utilize a variety of forces (for, e.g., inertial lift forces (wall effect and shear gradient forces) or hydrodynamic forces) to focus the cells within the flow into a streamline of cells. In some examples, the cells are focused in a single streamline. In some examples, the cells are focused in multiple streamlines, for example at least about 2, for example at least about 3, at least about 4, at least about 5, at least about 6, at least about 7, at least about 8, at least about 9, or at least about 10, or more, streamlines.

The focusing region receives a flow of randomly arranged cells using an upstream section. The cells flow into a region of contracted and expanded sections in which the randomly arranged cells are focused into a single streamline of cells. The focusing may be driven by the action of inertial lift forces (wall effect and shear gradient forces) acting on cells.

In some examples, the focusing region is formed with curvilinear walls that form periodic patterns. In some examples, the patterns form a series of square expansions and contractions. In other examples, the patterns are sinusoidal. In further examples, the sinusoidal patterns are skewed to form an asymmetric pattern. The focusing region may be effective in focusing cells over a wide range of flow rates. In the illustrated example, an asymmetrical sinusoidal-like structure is used as opposed to square expansions and contractions. This helps prevent the formation of secondary vortices and secondary flows behind the particle flow stream. In this way, the illustrated structure allows for faster and more accurate focusing of cells to a single lateral equilibrium position. Spiral and curved channels may also be used in an inertia regime; however, these may complicate the integration with other modules. Finally, straight channels where channel width is greater than channel height may also be used for focusing cells onto single lateral position. However, in this case, since there will be more than one equilibrium position in the z-plane, imaging may become problematic, as the imaging focal plane is preferably fixed. As may readily be appreciated, any of a variety of structures that provide a cross section that expands and contracts along the length of the microfluidic channel or are capable of focusing the cells may be utilized as appropriate to the requirements of specific applications.

The cell sorting system may be configured to focus the cell at a width and/or a height within the flow channel along an axis of the flow channel. The cell may be focused to a center or off the center of the cross-section of the flow channel. The cell may be focused to a side (e.g., a wall) of the cross-section of the flow channel. A focused position of the cell within the cross-section of the channel may be uniform or non-uniform as the cell is transported through the channel.

While specific implementations of focusing regions within microfluidic channels are described above, any of a variety of channel configurations that focus cells into a single streamline may be utilized as appropriate to the requirements of a specific application in accordance with various examples of the disclosure.

Microfluidic channels may be designed to impose ordering upon a single streamline of cells formed by a focusing region in accordance with several examples of the disclosure. Microfluidic channels in accordance with some examples of the disclosure include an ordering region having pinching regions and curved channels. The ordering region orders the cells and distances single cells from each other to facilitate imaging. In some examples, ordering is achieved by forming the microfluidic channel to apply inertial lift forces and Dean drag forces on the cells.

Different geometries, orders, and/or combinations may be used. In some examples, pinching regions may be placed downstream from the focusing channels without the use of curved channels. Adding the curved channels may help with more rapid and controlled ordering, as well as increasing the likelihood that particles follow a single lateral position as they migrate downstream. As may readily be appreciated, the specific configuration of an ordering region is largely determined based upon the requirements of a given application.

Architecture of the microfluidic channels of the cartridge of the present disclosure may be controlled (e.g., modified, optimized, etc.) to modulate cell flow along the microfluidic channels. Examples of the cell flow may include (i) cell focusing (e.g., into a single streamline) and (ii) rotation of the one or more cells as the cell(s) are migrating (e.g., within the single streamline) down the length of the microfluidic channels. In some examples, microfluidic channels may be configured to impart rotation on ordered cells in accordance with a number of examples of the disclosure. One or more cell rotation regions (e.g., the cell rotation region 836) of microfluidic channels in accordance with some examples of the disclosure use co-flow of a particle-free buffer to induce cell rotation by using the co-flow to apply differential velocity gradients across the cells. In some examples, a cell rotation region may introduce co-flow of at least about 1, for example at least about 2, at least about 3, at least about 4, at least about 5, or more buffers (e.g., particle-free, or containing one or more particles, such as polymeric or magnetic particles) to impart rotation on one or more cells within the channel. In some examples, a cell rotation region may introduce co-flow of at most about 5, for example, at most about 4, at most about 3, at most about 2, or about 1 buffer to impart the rotation of one or more cells within the channel. In some examples, the plurality of buffers may be co-flown at a same position along the length of the cell rotation region, or sequentially at different positions along the length of the cell rotation region. In some examples, the plurality of buffers may be the same or different. In several examples, the cell rotation region of the microfluidic channel is fabricated using a two-layer fabrication process so that the axis of rotation is perpendicular to the axis of cell downstream migration and parallel to cell lateral migration.

Cells may be imaged in at least a portion of the cell rotating region, while the cells are tumbling and/or rotating as they migrate downstream. In another example, the cells may be imaged in an imaging region that is adjacent to or downstream of the cell rotating region. In some examples, the cells may be flowing in a single streamline within a flow channel, and the cells may be imaged as the cells are rotating within the single streamline. A rotational speed of the cells may be constant or varied along the length of the imaging region. This may allow for the imaging of a cell at different angles (e.g., from a plurality of images of the cell taken from a plurality of angles due to rotation of the cell), which may provide more accurate information concerning cellular features than may be captured in a single image or a sequence of images of a cell that is not rotating to any significant extent. This also allow a 3D reconstruction of the cell using available software since the angles of rotation across the images are known. In another example, every single image of the sequence of image many be analyzed individually to analyze (e.g., classify) the cell from each image. In some examples, results of the individual analysis of the sequence of images may be aggregated to determine a final decision (e.g., classification of the cell).

In some examples, a cell rotation region of a microfluidic channel incorporates an injected co-flow prior to an imaging region in accordance with an example of the disclosure. Co-flow may be introduced in the z plane (perpendicular to the imaging plane) to spin the cells. Since the imaging is done in the x-y plane, rotation of cells around an axis parallel to the y-axis provides additional information by rotating portions of the cell that may have been occluded in previous images into view in each subsequent image. Due to a change in channel dimensions, at point xo, a velocity gradient is applied across the cells, which may cause the cells to spin. The angular velocity of the cells depends on channel and cell dimensions and the ratio between Q1 (main channel flow rate) and Q2 (co-flow rate) and may be configured as appropriate to the requirements of a given application. In some examples, a cell rotation region incorporates an increase in one dimension of the microfluidic channel to initiate a change in the velocity gradient across a cell to impart rotation onto the cell. In some examples, a cell rotation region of a microfluidic channel incorporates an increase in the z-axis dimension of the cross section of the microfluidic channel prior to an imaging region in accordance with an example of the disclosure. The change in channel height may initiate a change in velocity gradient across the cell in the z axis of the microfluidic channel, which may cause the cells to rotate as with using co flow.

In some examples, the system and methods of the present disclosure focuses the cells in microfluidic channels. The term focusing as used herein broadly means controlling the trajectory of cell/cells movement and comprises controlling the position and/or speed at which the cells travel within the microfluidic channels. In some examples controlling the lateral position and/or the speed at which the particles travel inside the microfluidic channels, allows to accurately predict the time of arrival of the cell at a bifurcation. The cells may then be accurately sorted. The parameters critical to the focusing of cells within the microfluidic channels include, but are not limited to channel geometry, particle size, overall system throughput, sample concentration, imaging throughput, size of field of view, and method of sorting.

In some examples the focusing is achieved using inertial forces. In some examples, the system and methods of the present disclosure focus cells to a certain height from the bottom of the channel using inertial focusing. In these examples, the distance of the cells from the objective is equal and images of all the cells will be clear. As such, cellular details, such as nuclear shape, structure, and size appear clearly in the outputted images with minimal blur. In some examples, the system disclosed herein has an imaging focusing plane that is adjustable. In some examples, the focusing plane is adjusted by moving the objective or the stage. In some examples, the best focusing plane is found by recording videos at different planes and the plane wherein the imaged cells have the highest Fourier magnitude, thus, the highest level of detail and highest resolution, is the best plane.

In some examples, the system and methods of the present disclosure utilize a hydrodynamic-based z focusing system to obtain a consistent z height for the cells of interests that are to be imaged. In some examples, the design comprises hydrodynamic focusing using multiple inlets for main flow and side flow. In some examples, the hydrodynamic-based z focusing system is a triple-punch design. In some examples, the design comprises hydrodynamic focusing with three inlets, wherein the two side flows pinch cells at the center. For certain channel designs, dual z focus points may be created, wherein a double-punch design similar to the triple-punch design may be used to send objects to one of the two focus points to get consistent focused images. In some examples, the design comprises hydrodynamic focusing with 2 inlets, wherein only one side flow channel is used and cells are focused near channel wall. In some examples, the hydrodynamic focusing comprises side flows that do not contain any cells and a middle inlet that contains cells. The ratio of the flow rate on the side channel to the flow rate on the main channel determines the width of cell focusing region. In some examples, the design is a combination of the above. In all examples, the design is integrable with the bifurcation and sorting mechanisms disclosed herein. In some examples, the hydrodynamic-based z focusing system is used in conjunction with inertia-based z focusing.

In some examples, the cell is a live cell. In some examples, the cell is a fixed cell (e.g., in methanol or paraformaldehyde). In some examples, one or more cells may be coupled (e.g., attached covalently or non-covalently) to a substrate (e.g., a polymeric bead or a magnetic bead) while flowing through the cartridge. In some examples, the cell(s) may not be coupled to any substrate while flowing through the cartridge.

A variety of techniques may be utilized to classify images of cells captured by classification and/or sorting systems in accordance with various examples of the disclosure. In some examples, the image captures are saved for future analysis/classification either manually or by image analysis software. Any suitable image analysis software may be used for image analysis. In some examples, image analysis is performed using OpenCV. In some examples, analysis and classification is performed in real time.

In some examples, the system and methods of the present disclosure comprise collecting a plurality of images of objects in the flow. In some examples, the plurality of images comprises at least 20 images of cells. In some examples, the plurality of images comprises at least about 19, for example, about 18, about 17, about 16, about 15, about 14, about 13, about 12, about 11, about 10, about 9, about 8, about 7, about 6, about 5, about 4, about 3, or about 2 images of cells. In some examples, the plurality of images comprises images from multiple cell angles. In some examples, the plurality of images, comprising images from multiple cell angles, help derive extra features from the particle which would be hidden if the particle is imaged from a single point-of-view. In some examples, without wishing to be bound by any particular theory, the plurality of images, comprising images from multiple cell angles, help derive extra features from the particle which would be hidden if a plurality of images are combined into a multi-dimensional reconstruction (e.g., a two-dimensional hologram or a three-dimensional reconstruction).

In some examples, the systems and methods of present disclosure allow for a tracking ability, wherein the system and methods track a particle (e.g., cell) under the camera and maintain the knowledge of which frames belong to the same particle. In some examples, the particle is tracked until it has been classified and/or sorted. In some examples, the particle may be tracked by one or more morphological (e.g., shape, size, area, volume, texture, thickness, roundness, etc.) and/or optical (e.g., light emission, transmission, reflectance, absorbance, fluorescence, luminescence, etc.) characteristics of the particle. In some examples, each particle may be assigned a score (e.g., a characteristic score) based on the one or more morphological and/or optical characteristics, thereby to track and confirm the particle as the particle travels through the microfluidic channel.

In some examples, the systems and methods of the disclosure comprise imaging a single particle in a particular field of view of the camera. In some examples, the same instrument that performs imaging operations may also perform sorting operations. In some examples, the system and methods of the present disclosure image multiple particles (e.g., cells) in the same field of view of camera. Imaging multiple particles in the same field of view of the camera may provide additional advantages, for example it will increase the throughput of the system by batching the data collection and transmission of multiple particles. In some instances, at least about 2, for example at least about 3, at least about 4, at least about 5, at least about 6, at least about 7, at least about 8, at least about 9, at least about 10, at least about 20, at least about 30, at least about 40, at least about 50, at least about 60, at least about 70, at least about 80, at least about 90, at least about 100, or more particles are imaged in the same field of view of the camera. In some instances, about 100 to about 200 particles are imaged in the same field of view of the camera. In some instances, at most about 100, for example at most about 90, at most about 80, at most about 70, at most about 60, at most about 50, at most about 40, at most about 30, at most about 20, at most about 10, at most about 9, at most about 8, at most about 7, at most about 6, at most about 5, at most about 4, at most about 3, or at most about 2 particles are imaged in the same field of view of the camera. In some examples, the number of the particles (e.g., cells) that are imaged in the same field of view may not be changed throughout the operation of the cartridge. In another example, the number of the particles (e.g., cells) that are imaged in the same field of view may be changed in real-time throughout the operation of the cartridge, e.g., to increase speed of the classification and/or sorting process without negatively affecting quality or accuracy of the classification and/or soring process.

The imaging region maybe downstream of the focusing region and the ordering region. Thus, the imaging region may not be part of the focusing region and the ordering region. In an example, the focusing region may not comprise or be operatively coupled to any imaging device that is configured to capture one or more images to be used for particle analysis (e.g., cell classification).

In some examples, the systems and the methods of the present disclosure actively sorts a stream of particles. The term sort or sorting as used herein refers to physically separating particles, for, e.g., cells, with one or more desired characteristics. The desired characteristic(s) may comprise a morphometric feature of the cell(s) analyzed and/or obtained from the image(s) of the cell, or a combination of such morphometric features.

Examples of the morphometric feature(s) of the cell(s) may comprise a size, shape, volume, electromagnetic radiation absorbance and/or transmittance (e.g., fluorescence intensity, luminescence intensity, etc.), or viability (e.g., when live cells are used), or a morphometric feature selected from Table 1 or from Table 2.

In a manner such as described with reference to FIG. 2, the flow channel (corresponding to fluidic channel 213) may branch into a plurality of channels, and the cell sorting system may be configured to sort the cell by directing the cell to a selected channel of the plurality of channels based on the analyzed image of the cell. The analyzed image may be indicative of one or more features of the cell, wherein the feature(s) are used as parameters of cell sorting. In some examples, one or more channels of the plurality of channels may have a plurality of sub channels, and the plurality of sub-channels may be used to further sort the cells that have been sorted once.

Cell sorting may comprise isolating one or more target cells from a population of cells. The target cell(s) may be isolated into a separate reservoir that keeps the target cell(s) separate from the other cells of the population. Cell sorting accuracy may be defined as a proportion (e.g., a percentage) of the target cells in the population of cells that have been identified and sorted into the separate reservoir. In some examples, the cell sorting accuracy of the cartridge provided herein may be at least about 80%, for example at least about 90%, at least about 95%, at least about 98%, at least about 99%, or more (e.g., about 99.9% or more, or about 100%).

In some examples, cell sorting may be performed at a rate of at least about 1 cell/second, for example at least about 5 cells/second, at least about 10 cells/second, at least about 50 cells/second, at least about 100 cells/second, at least about 500 cells/second, at least about 1,000 cells/second, at least about 5,000 cells/second, at least about 10,000 cells/second, at least about 50,000 cells/second, or more. In some examples, cell sorting may be performed at a rate of at most about 50,000 cells/second, for example at most about 10,000 cells/second, at most about 5,000 cells/second, at most about 1,000 cells/second, at most about 500 cells/second, at most about 100 cells/second, at most about 50 cells/second, at most about 10 cells/second, at most about 5 cells/second, or at most about 1 cell/second, or less.

In some examples, the systems and methods disclosed herein use an active sorting mechanism. In various examples, the active sorting is independent from analysis and decision making platforms and methods. In various examples the sorting is performed by a sorter, which receives a signal from the decision making unit (e.g. a classifier), or any other external unit, and then sorts cells as they arrive at the bifurcation. The term bifurcation as used herein refers to the termination of the flow channel into two or more channels, such that cells with the one or more desired characteristics are sorted or directed towards one of the two or more channels and cell without the one or more desired characteristics are directed towards the remaining channels. In some examples, the flow channel terminates into at least about 2, for example at least about 3, at least about 4, at least about 5, at least about 6, at least about 7, at least about 8, at least about 9, at least about 10, or more channels. In some examples, the flow channel terminates into at most about 10, for example at most about 9, at most about 8, at most about 7, at most about 6, at most about 5, at most about 4, at most about 3, or at most about 2 channels. In some examples, the flow channel terminates in two channels and cells with one or more desired characteristics are directed towards one of the two channels (the positive channel), while cells without the one or more desired characteristics are directed towards the other channel (the negative channel). In some examples, the flow channel terminates in three channels and cells with a first desired characteristic are directed to one of the three channels, cells with a second desired characteristic are directed to another of the three channels, and cells without the first desired characteristic and the second desired characteristic are directed to the remaining of the three channels.

In some examples, the sorting is performed by a sorter. The sorter may function by predicting the exact time at which the particle will arrive at the bifurcation. To predict the time of particle arrival, the sorter may use any applicable method. In some examples, the sorter predicts the time of arrival of the particle by using (i) velocity of particles (e.g., downstream velocity of a particle along the length of the microfluidic channel) that are upstream of the bifurcation and (ii) the distance between velocity measurement/calculation location and the bifurcation. In some examples, the sorter predicts the time of arrival of the particles by using a constant delay time as an input.

In some examples, prior to the cell's arrival at the bifurcation, the sorter may measure the velocity of a particle (e.g., a cell) at least about 1, for example at least about 2, at least about 3, at least about 4, or at least about 5, or more times. In some examples, prior to the cell's arrival at the bifurcation, the sorter may measure the velocity of the particle at most about 5, for example at most about 4, at most about 3, at most about 2, or at most about 1 time. In some examples, the sorter may use at least about 1, for example at least about 2, at least about 3, at least about 4, or at least about 5, or more sensors. In some examples, the sorter may use at most about 5, for example at most about 4, at most about 3, at most about 2, or at most about 1 sensor. Example of the sensor(s) may be an imaging device (e.g., a camera such as a high-speed camera), one- or multi-point light (e.g., laser) detector, etc. Referring to FIGS. 8A-8B the sorter may use any one of the imaging devices (e.g., the high-speed camera system 814) disposed at or adjacent to the imaging region 838. In some examples, the same imaging device(s) may be used to capture one or more images of a cell as the cell is rotating and migrating within the channel, and the one or more images may be analyzed to (i) classify the cell and (ii) measure a rotational and/or lateral velocity of the cell within the channel and predict the cell's arrival time at the bifurcation. In some examples, the sorter may use one or more sensors that are different than the imaging devices of the imaging region 838. The sorter may measure the velocity of the particle (i) upstream of the imaging region 838, (ii) at the imaging region 838, and/or (iii) downstream of the imaging region 838.

The sorter may comprise or be operatively coupled to a processor, such as a computer processor. Such processor may be the processor 816 that is operatively coupled to the imaging device 814 or a different processor. The processor may be configured to calculate the velocity of a particle (rotational and/or downstream velocity of the particle) an predict the time of arrival of the particle at the bifurcation. The processor may be operatively coupled to one or more valves of the bifurcation. The processor may be configured to direct the valve(s) to open and close any channel in fluid communication with the bifurcation. The processor may be configured to predict and measure when operation of the valve(s) (e.g., opening or closing) is completed.

In some examples, the sorter may comprise a self-included unit (e.g., comprising the sensors, such as the imaging device(s)) which is capable of (i) predicting the time of arrival of the articles and/or (ii) detecting the particle as it arrives at the bifurcation. In order to sort the particles, the order at which the particles arrive at the bifurcation, as detected by the self-included unit, may be matched to the order of the received signal from the decision making unit (e.g. a classifier). In some examples, controlled particles are used to align and update the order as necessary. In some examples, the decision making unit may classify a first cell, a second cell, and a third cell, respectively, and the sorter may confirm that the first cell, the second cell, and the third cell are sorted, respectively in the same order. If the order is confirmed, the classification and sorting mechanisms (or deep learning algorithms) may remain the same. If the order is different between the classifying and the sorting, then the classification and/or sorting mechanisms (or deep learning algorithms) may be updated or optimized, either manually or automatically. In some examples, the controlled particles may be cells (e.g., live or dead cells). In some examples, the controlled particles may be special calibration beads (e.g., plastic beads, metallic beads, magnetic beads, etc.).

In some examples, the sorter (or an additional sensor disposed at or adjacent to the bifurcation) may be configured to validate arrival of the particles (e.g., the cells) at the bifurcation. In some examples, the sorter may be configured to measure an actual arrival time of the particles (e.g., the cells) at the bifurcation. The sorter may analyze (e.g., compare) the predicted arrival time, the actual arrival time, the velocity of the particles downstream of the channel prior to any adjustment of the velocity, and/or a velocity of the particles downstream of the channel subsequent to such adjustment of the velocity. Based on the analyzing, the sorter may modify any operation (e.g., cell focusing, cell rotation, controlling cell velocity, cell classification algorithms, valve actuation processes, etc.) of the cartridge. The validation by the sorter may be used for closed-loop and real-time update of any operation of the cartridge.

In some examples, to predict the time of arrival of one or more cells for sorting, the systems, methods, and platforms disclosed herein may dynamically adjust a delay time (e.g., a constant delay time) based on imaging of the cell(s) or based on tracking of the cell(s) with light (e.g., laser). By detecting changes (e.g., flow rates, velocity of aggregate of multiple cells, the lateral location of cells in the channel, etc.) the delay time (e.g., time at which the cells arrive at the bifurcation) may be predicted and adjusted in real-time (e.g., every few milliseconds). A feedback loop may be designed that may constantly read such changes and adjust the delay time accordingly. In another example, the delay time may be adjusted for each cell/particle. The delay time may be calculated separately for each individual cell, based on, e.g., its velocity, lateral position in the channel, and/or time of arrival at specific locations along the channel (e.g., using tracking based on lasers or other methods). The calculated delay time may then be applied to the individual cell/particle (e.g., if the cell is a positive cell or a target cell, the sorting may be performed according to its specific delay time or a predetermined delay time). In some examples, the sorters used in the systems and methods disclosed herein are self-learning cell sorting systems or intelligent cell sorting systems, as disclosed herein.

These sorting systems may continuously learn based on the outcome of sorting. For example, a sample of cells is sorted, the sorted cells are analyzed, and the results of this analysis are fed back to the classifier. In some examples, the cells that are sorted as “positive” (i.e., target cells or cells of interest) may be analyzed and validated. In some examples, the cells that are sorted as “negative” (i.e., non-target cells or cells not of interest) may be analyzed and validated. In some examples, both positive and negative cells may be validated. Such validation of sorted cells (e.g., based on secondary imaging and classification) may be used for closed-loop and real-time update of the primary cell classification algorithms.

In some examples, a flush mechanism may be used during sorting. The flush mechanism may ensure that the cell which has been determined to be sorted to a specific bucket or well will end up there (e.g., not be stuck in various parts of the channel or outlet). The flush mechanism may ensure that the channel and outlets stay clean and debris-free for maximum durability. The flush mechanism may inject additional solutions/reagents (e.g., cell lysis buffers, barcoded reagents, etc.) to the well or droplet that the cell is being sorted into. The flush mechanism may be supplied by a separate set of channels and/or valves which are responsible to flow a fluid at a predefined cadence in the direction of sorting.

In some examples, the methods and systems disclosed herein may use any sorting technique to sort particles. At least a portion of the collection reservoir may or may not be pre filled with a fluid, e.g., a buffer. In some examples, the sorting technique comprises closing a channel on one side of the bifurcation to collect the desired cell on the other side. In some examples, the closing of the channels may be carried out by employing any known technique. In some examples, the closing is carried out by application of a pressure. In some instances, the pressure is pneumatic actuation. In some examples, the pressure may be positive pressure or negative pressure. In some examples, positive pressure is used. In some examples, one side of the bifurcation is closed by applying pressure and deflecting the soft membrane between top and bottom layers. Other examples of systems and methods of particle (e.g., cell) imaging, analysis, and sorting are further described in International Application No. PCT/US2017/033676 and International Application No. PCT/US2019/046557, each of which is incorporated herein by reference in its entirety.

In various examples, the systems and methods of the present disclosure comprise one or more reservoirs designed to collect the particles after the particles have been sorted. In some examples, the number of cells to be sorted is about 1 cell to about 1,000,000 cells. In some examples, the number of cells to be sorted is at least about 1 cell. In some examples, the number of cells to be sorted is at most about 1,000,000 cells. In some examples, the number of cells to be sorted is about 1 cell to about 100 cells, about 1 cell to about 1,000 cells, about 1 cell to about 10,000 cells, about 1 cell to about 100,000 cells, about 1 cell to about 1,000,000 cells, about 100 cells to about 1,000 cells, about 100 cells to about 10,000 cells, about 100 cells to about 100,000 cells, about 100 cells to about 1,000,000 cells, about 1,000 cells to about 10,000 cells, about 1,000 cells to about 100,000 cells, about 1,000 cells to about 1,000,000 cells, about 10,000 cells to about 100,000 cells, about 10,000 cells to about 1,000,000 cells, of about 100,000 cells to about 1,000,000 cells. In some examples, the number of cells to be sorted is about 1 cell, for example about 100 cells, about 500 cells, about 1,000 cells, about 5,000 cells, about 10,000 cells, about 50,000 cells, about 100,000 cells, about 500,000 cells, or about 1,000,000 cells or more. In some examples, the number of cells to be sorted is about 100 to about 500 cells, about 200 to about 500 cells, about 300 to about 500 cells, about 350 to about 500 cells, about 400 to about 500 cells, or about 450 to about 500 cells. In some examples, the reservoirs may be milliliter scale reservoirs. In some examples, the one or more reservoirs are pre-filled with a buffer and the sorted cells are stored in the buffer. Using the buffer helps to increase the volume of the cells, which may then be easily handled, for example a pipetted. In some examples, the buffer is a phosphate buffer, for example phosphate-buffered saline (PBS).

In some examples, the system and methods of the present disclosure comprise a cell sorting technique wherein pockets of buffer solution containing no negative objects are sent to the positive output channel in order to push rare objects out of the collection reservoir. In some examples, additional buffer solution is sent to the positive output channel to flush out all positive objects at the end of a run, once the channel is flushed clean (e.g., using the flush mechanism as disclosed herein).

In some examples, the system and methods of the present disclosure comprise a cell retrieving technique, wherein sorted cells may be retrieved for downstream analysis (e.g., molecular analysis). Non-limiting examples of the cell retrieving technique may include: retrieval by centrifugation; direct retrieval by pipetting; direct lysis of cells in well; sorting in a detachable tube; feeding into a single cell dispenser to be deposited into 96 or 384 well plates; etc.

In some examples, the system and methods of the present disclosure comprise a combination of techniques, wherein a graphics processing unit (GPU) and a digital signal processor (DSP) are used to run artificial intelligence (AI) algorithms and apply classification results in real-time to the system. In some examples, the system and methods of the present disclosure comprise a hybrid method for real-time cell sorting.

In some examples, the system and methods of the present disclosure comprise a feedback loop (e.g., an automatic feedback loop). For example, the system and methods may be configured to (i) monitor the vital signals and (ii) finetune one or more parameters of the system and methods based on the signals being read. At the beginning or throughout the run (e.g., the use of the microfluidic channel for cell imaging, classification, and/or sorting), a processor (e.g., a ML/AI processor as disclosed herein) may specify target values for one or more selected parameters (e.g., flow rate, cell rate, etc.). In another example, other signals that reflect (e.g., automatically reflect) the quality of the run (e.g., the number of cells that are out of focus within the last 100 imaged cells) may be utilized in the feedback loop. The feedback loop may receive (e.g., in real-time) values of the parameters/signals disclosed herein and, based on the predetermined target values and/or one or more general mandates (e.g., the fewer the out-of-focus cells, the better), the feedback loop may facilitate adjustments (e.g., adjustments to pressure systems, illumination, stage, etc.). In some examples, the feedback loop may be designed to monitor and/or handle degenerate scenarios, in which the microfluidic system is not responsive or malfunctioning (e.g., outputting a value read that is out of range of acceptable reads).

In some examples, the system and methods of the present disclosure may adjust a cell classification threshold based on expected true positive rate for a sample type. The expected true positive rate may come from statistics gathered in one or more previous runs from the same or other patients with similar conditions. Such approach may help neutralize run-to-run variations (e.g., illumination, chip fabrication variation, etc.) that would impact imaging and hence any inference therefrom.

In some examples, the systems disclosed herein further comprise a validation unit that detects the presence of a particle without getting detailed information, such as imaging. In some instances, the validation unit may be used for one or more purposes. In some examples, the validation unit detects a particle approaching the bifurcation and enables precise sorting. In some examples, the validation unit detects a particle after the particle has been sorted to one of subchannels in fluid communication with the bifurcation. In some examples, the validation unit provides timing information with a plurality of laser spots, e.g., two laser spots. In some instances, the validation unit provides timing information by referencing the imaging time. In some instances, the validation unit provides precise time delay information and/or flow speed of particles.

In some examples, the particles (e.g., cells) analyzed by the systems and methods disclosed herein are comprised in a sample. The sample may be a biological sample obtained, directly or indirectly, from a subject (e.g., a human or any animal). It should be appreciated that an animal may be a variety of any applicable type, including, but not limited thereto, mammal or non-mammals. For example, the animal may be veterinarian animal, livestock animal or pet type animal, etc. As an example, the animal may be a laboratory animal specifically selected to have certain characteristics similar to a human (e.g., rat, dog, pig, monkey, or the like). It should be appreciated that the subject may be any applicable human patient, for example. In some examples, the biological sample comprises, or is derived from, a biopsy sample from a subject.

In some examples, the sample may be, or may be derived from, a liquid biological sample. In some examples, the liquid biological sample may be a blood sample (e.g., whole blood, plasma, or serum). A whole blood sample may be subjected to separation of cellular components (e.g., plasma, serum) and cellular components by use of a Ficoll reagent. In some examples, the liquid biological sample may be a urine sample. In some examples, the liquid biological sample may be a perilymph sample. In some examples, the liquid biological sample may be a fecal sample. In some examples, the liquid biological sample may be saliva. In some examples, the liquid biological sample may be semen. In some examples, the liquid biological sample may be amniotic fluid. In some examples, the liquid biological sample may be cerebrospinal fluid. In some examples, the liquid biological sample may be bile. In some examples, the liquid biological sample may be sweat. In some examples, the liquid biological sample may be tears. In some examples, the liquid biological sample may be sputum. In some examples, the liquid biological sample may be synovial fluid. In some examples, the liquid biological sample may be vomit.

In some examples, samples may be collected over a period of time and the samples may be compared to each other or with a standard sample using the systems and methods disclosed herein. In some examples the standard sample is a comparable sample obtained from a different subject, for example a different subject that is known to be healthy or a different subject that is known to be unhealthy. Samples may be collected over regular time intervals, or may be collected intermittently over irregular time intervals.

### Cell Feature Extraction: Output Data and Interpretation

FIG. 9 illustrates an example training architecture 900 for aspects herein disclosed systems, such as the human foundation model previously illustrated in FIG. 1. Architecture 900 may be completely symmetric, completely asymmetric, partially symmetric, or partially asymmetric, or a combination of the foregoing, and include a multi-layered (e.g., 18 layers deep) convolutional neural network. It is understood that after training, the system (e.g., system 100) may be trained to predict morphometric characteristics of cellular images, including but not limited to cell class, cell type, cell state, other morphometric features such as blobs, related probabilities, and related accuracy identifiers.

In some examples, architecture 900 may be deployed on instruments (e.g., REM=I instrument of Table 4) and may be used to generate embeddings in a cloud-based computer system. Architecture 900 may be used in a high-throughput setting so that images 912. In some examples, images 912 are captured by a camera (e.g., an ultra high-speed bright-field camera) as cell suspensions flow through a channel in the microfluidics chip. Architecture 900 may include an augmentation module 940 configured to crop collected ultra-high-speed bright-field images 912 of cells as they pass through an imaging zone (e.g., an imaging zone of a microfluidic chip such as those captured images of FIGS. 8A-8B). Augmentation module 940 may implement one or more augmentation methods to generate batches 942a, b of altered replicas of the images 912. Augmentation techniques of module 940 includes, but is not limited to, horizontal and vertical flips of images, orthogonal rotation, translation, gaussian noise, contrast variation, and the like.

Batches 942a, b may be used to train a deep learning (DL) encoder 950. Specifically, batches 942a, b of altered replicas of the images 912 may be introduced along with images 912 into DL encoder 950 to generate augmented embeddings 952a, b. Encoder 950 may be trained using a self-supervised learning (SSL) method that learns image features without labels and relies at least on preserving information of its embeddings, including embeddings 952a, 952b, as well as concatenated deep learning predictive embeddings 964 (discussed more particularly below). DL encoder 950 may be a ResNet based encoder trained using a plurality of unlabeled cell images from different types of samples to detect differences in cell morphology without labeled training data. In some examples, encoder 950 learns image features without labels and with orthogonal morphometric features to improve model performance and interpretability.

Encoder 950 may include a plurality of convolution layers that use examples, such as edge detectors to detect a plurality of edge components of images 912 and batches 942a, b of altered replicas of the images 912. Encoder 950 may also use shape detectors to detect shape components of images 912 and batches 942a, b of altered replicas of the images 912 (e.g., a particular type of cell ridge). Augmented embeddings 952a, b from deep learning encoder 950 may be used to determine deep learning interpretations of captured images 912 performed in real-time (e.g., approximately <150 ms latency). To generate embeddings 952a, b, encoder 950 may encode features of batches 942a, b into multi-dimensional feature vectors. In some examples, encoder 950 may extract a 64-dimensional feature vector for each altered image of batches 942a, b and images 912.

Encoder 950 may be trained with a loss function that utilizes maximum likelihood-based invariance between augmented images (such as mean squared error or categorical cross entropy), as well as variance, covariance, and morphometric decorrelation terms. In some examples, the variance and covariance terms used herein may include estimates of variance and covariance between feature dimensions by calculating the invariance and covariance directly for batches of images (e.g., of hundreds to thousands of images). In some examples, encoder 950 may be iteratively optimized until the DL model converges and calculate statistical quality (e.g., covariance) using the loss function. In some examples, encoder 950 may include a backbone, such as a ResNet-50 backbone, trained with the herein described invariance, variance, covariance, and morphometric decorrelation terms. The loss function uses an invariance term that learns invariance to vector transformations and is regularized with a variance term that prevents norm collapse. In some examples, the invariance term is determined using the mean square distance between embedding vectors (e.g., vectors of embeddings 952a, 952b). The loss function also uses a covariance term that prevents informational collapse by decorrelating the different dimensions of the vectors of embeddings 952a, 952b. The variance loss constrains the variance term of the vectors of embeddings 952a. 952b along each dimension independently. To determine similarity loss between vectors of embeddings 952a, 952b, a distance between vector pairs of embeddings 952a, b of the augmented images of batches 942a, b of the same cell is minimized (e.g., Euclidean distance) and variance of each embedding 952a, b over a training batch is maintained above a threshold. In some examples, the threshold is a hyperparameter determined by a value that gives us the best or most-optimized performance on downstream tasks. In some examples, variance is optimized to be around approximately 1. In some examples, variance may be optimized to be any value on a range of 0 or strictly (0, infinity] (strictly greater than 0).

With respect to the covariance term, different dimensions of embeddings 952a, 952b are used to make non-diagonal values of a cross-correlation matrix to zero indicating that the values are orthogonal. The covariances between embedding variables 952a, b over a respective batch between every pair of centered embedding variable is attracted to zero (e.g., see each of embeddings 952a, 952b) so that embeddings 952a, b are decorrelated from each other.

Architecture 900 may also include a computer vision encoder 960 that may be self-supervised and may include human-constructed algorithms, which in some cases may be referred to as the previously-described “rule-based morphometrics.” See Table 1. Encoder 960 may process captured images 912 as input and extract morphometric cell features into a plurality of morphometric vectors 962 (e.g., dimensional morphometric features encoded into 95-dimensional feature vectors representing the cell morphology). The multi-dimension vectors 962 may include cell position, cell shape, pixel intensity, pixel count, cell size, texture, focus, or combinations thereof. In one nonlimiting example, encoder 960 may extract 99 dimensional embedding vectors representing cell morphology from high resolution images 912. In one nonlimiting example, previously described 64-dimensional embeddings 952a, b, and 51-dimensional morphometric features 962 may be encoded into 95-dimensional feature vectors representing the cell morphology.

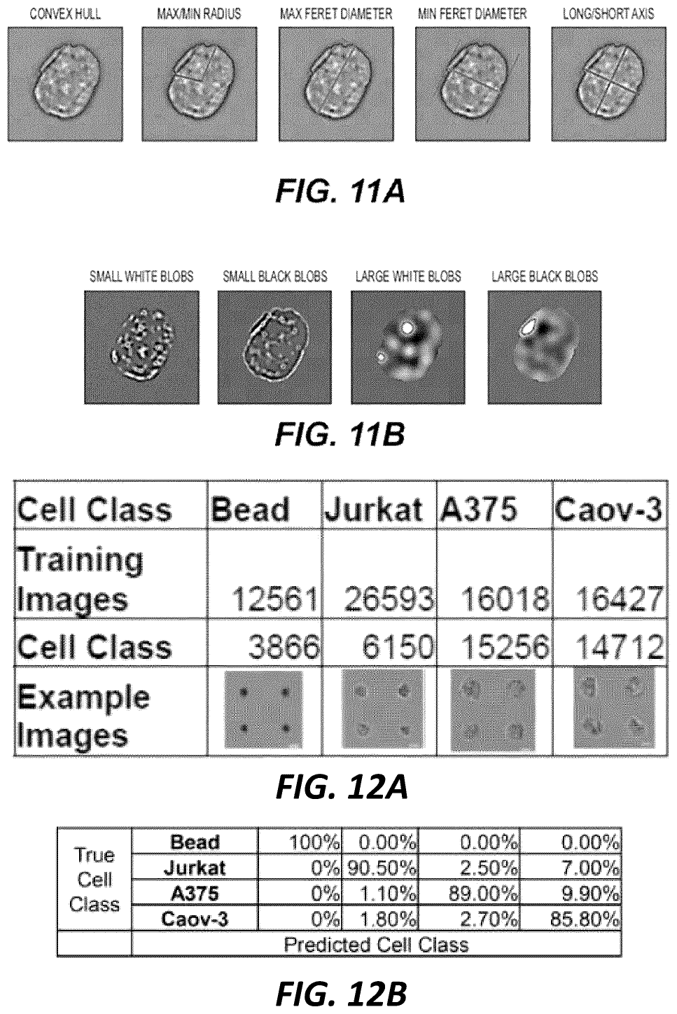

Example depictions of certain contemplated morphometric cell features are shown in FIGS. 11A to 11B, where FIG. 11A illustrates representative images showing features that include cell shape and size (e.g., convex hull, max/min radius, max ferret diameter, min ferret diameter, long/short axis, etc.). FIG. 11B shows representative images showing features that include pixel intensity and texture (e.g., small white “blobs”, small black “blobs”, large white “blobs”, large white “blobs”, etc.). The illustrated morphometrics that describe size and intensities of “blobs” relate to cellular structures like granules, vesicles, and the like. In some examples, “blobs” may be understood as connected set(s) of pixels that are either substantially or entirely dark or substantially or entirely bright. In some examples, “blobs” may be understood region(s) in a respective image that differs in properties (e.g., brightness, color, etc.) relative to surrounding region(s).

In some examples, outputs of encoders 950, 960 may be analyzed together and concatenate as decorrelated concatenated morphometric predictive embeddings 964. Embeddings 964 may be generated adopting a probabilistic approach and/or using deep learning features of encoder 950 (e.g., using conditional batch normalization) concatenated with computer vision morphometric feature embeddings 962 from encoder 960 into different dimensions. Embeddings 964 may be predictive multidimensional feature vectors that include predictive features related to individual cells, clusters of cells, morphometric features, and related probabilities.

Embeddings 964 may be generated using morphometric decorrelation with encoder 950 minimizing the covariance term between vector pairs of embeddings 962 and rule-based morphometric dimensions over a training batch. Embeddings 964 may include encoded novel cell morphology information retained to be orthogonal to the rule-based morphometrics of encoder 960, including “blob” features such as those shown in FIGS. 11A to 11B. In so doing, architecture 900 effectively uses encoder 950 to splits its embedding layer into multiple sections to separate morphometric de-correlated embeddings 962 and morphometrics predictive embeddings 964 to predict or otherwise approximate morphometric features, such as “blobs”. In so splitting, architecture 900 is able to optimize conflict objectives in different parameter space and thus avoids adding latency to the system. In some examples, a correlation coefficient of predicted and/or approximated morphometric features, such as “blob” features, may be greater than approximately 0.9, though other ranges are contemplated as needed or required, including approximately 0.85 to 0.95.

In FIG. 10, architecture 900 is continued from FIG. 9 and shows that in one example a multi-layer projector 970 may be included downstream of encoder 950. In this example, projector 970 is provided to project high dimension to low dimension (e.g., 2D), such as reducing the dimensionality of the embeddings 952a, 952b. In this respect, embeddings 952a, 952b are capable of being visualized. Projector 970 is configured to reduce the dimensionality to projected embeddings 972a, 972b and map representations of embeddings 952a, 952b. In this example, the previously discussed criterion, including the loss functions with invariance, variance, covariance, and morphometric decorrelation terms may be applied on projected embeddings 972a, 972b.

FIG. 12A illustrates cell classes, numbers of images used as training dataset to train a classifier using features extracted using the human foundation model, numbers of images processed by the human foundation model as test dataset, and corresponding representative cell images, in accordance with some examples of the present disclosure. A scale bar of 10 μm is shown on the representative cell images.

FIG. 12B illustrates a confusion matrix between predicted cell classes classified using features extracted using the human foundation model and actual cell classes, in accordance with some examples of the present disclosure. As illustrated, the human foundation model predicts cell classes with a high accuracy. The accuracy for predicting the Jurkat cell line, A375 cell line, and Caov-3 cell line is 90.5%, 89%, and 95.8%, respectively. The accuracy for predicting polystyrene beads is 100%.

### Classifying and Sorting

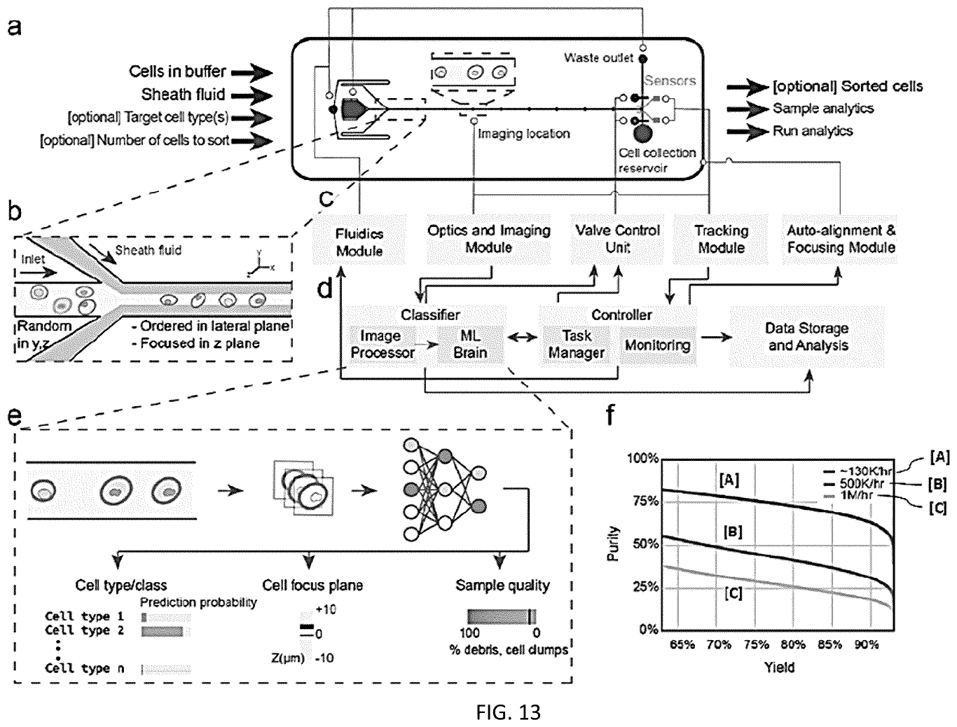

Views (a) to (f) of FIG. 13 schematically illustrate an example system for classifying and sorting one or more cells. The platform as disclosed herein may allow for the input and flow of cells in suspension with confinement along a single lateral trajectory to obtain a narrow band of focus across the z-axis (views (a) to (f) of FIG. 13). View (a) of FIG. 13 shows the microfluidic chip and the inputs and output of the sorter platform according to one example of the present disclosure. Cells in suspension and sheath fluid are inputted, along with run parameters entered by the user: target cell type(s) and a cap on the number of cells to sort, if sorting is of interest. Upon run completion, the system generates reports of the sample composition (number and types of all of the processed cells) and the parameters of the run, including: length of run, number of analyzed cells, quality of imaging, quality of the sample. If sorting option is selected, it outputs isolated cells in a reservoir on the chip as well as a report of the number of sorted cells, purity of the collected cells and yield of the sort. Referring to view (b) of FIG. 13, a combination of hydrodynamic focusing and inertial focusing is used to focus the cells on a single z plane and a single lateral trajectory. Referring to views (c) and (d) of FIG. 13, the diagram shows the interplay between different components of the software (view (c) of FIG. 13) and hardware pieces (view (d) of FIG. 13). The classifier is blown up in view (e) of FIG. 13, depicting the process of image collection, and automated real-time assessment of single cells in flow. After the images are taken, individual cell images are cropped using an automated object detection module, the cropped images are then run through a deep neural networks model trained on the relevant cells (e.g., DL encoder 950). For each image, the model may generate deep learning embeddings (e.g., embeddings 952a. 952b), deep learning predictive embeddings 964, as well as generate a prediction vector over the available cell classes and an inference will be made according to a selection rule (e.g., argmax). The model may also infer the z focusing plane of the image. The percentage of debris and cell clumps may also be predicted by the neural network model as a proxy for “sample quality”. View (f) of FIG. 13 shows the performance of sorting. In this figure, the tradeoff between purity and yield is shown in three different modes, for profiling as sorting of 130,000 [A], 500,000 [B] or 1,000,000 [C] cells within one hour.

Using a combination of hydrodynamic and inertial focusing, the platform may collect ultra high-speed bright-field images of cells as they pass through the imaging zone of the microfluidic chip (views (a) and (b) of FIG. 13). In order to capture the single cell images for processing, an automated object detection module may be incorporated to crop each image centered around the cell, before feeding the cropped images into a deep convolutional neural network (CNN) based on Inception architecture, which is trained on images of relevant cell types. In addition to classifying cells into categories of interest, the CNN may be trained to assess the focus of each image (in Z plane) and identify debris and cell clusters, thus providing information to assess sample quality (view (c) of FIG. 13). A feedback loop may be engineered so that the CNN inferred cell type may be used in real time to regulate pneumatic valves for sorting a cell into either the positive reservoir (cell collection reservoir) for a targeted category of interest or a waste outlet (FIG. 13A). Sorted cells in the reservoir may then be retrieved for downstream processing and molecular analysis. In some examples, the feedback loop may be engineered so that the generated deep learning embeddings (e.g., embeddings 952a, 952b, 964, etc.) may be used in real time to regulate pneumatic valves for sorting a cell into either a cell collection reservoir or a waste outlet (FIG. 13A).

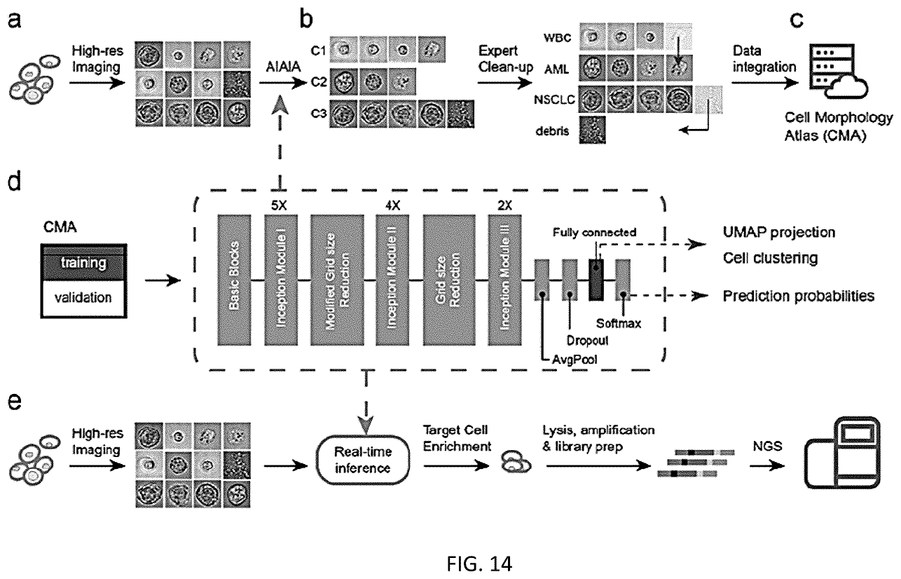

FIG. 14 schematically illustrate operations that may be performed in an example method. View (a) of FIG. 14 shows high resolution images of single cells in flow are stored. Referring to view (b) of FIG. 14, AIAIA (AI Assisted Image Annotation) is used to cluster individual cell images into morphologically similar groups of cells. In some examples, AIAIA is used to cluster individual cell images into groups of cells using deep learning embeddings (e.g., embeddings 952a, 952b, 964). In some examples, a user uses the labeling tool to adjust and batch-label the cell clusters. In the example shown, one AML cell may be mis-clustered into a group of WBC cells and an image showing a cell clump (debris) may be mis-clustered in a NSCLC cell group. These errors are corrected by the “Expert clean-up” operation of view (b). Referring to view (c) of FIG. 14, the annotated cells are then integrated into a Cell Morphology Atlas (CMA). Referring to view (d) of FIG. 14, the CMA is used to generate both training and validation sets of the next generation of the models. Referring to view (e) of FIG. 14, during a sorting experiment, the pre-trained model shown in view (d) of FIG. 14 is used to infer the cell type (class) in real-time. The enriched cells are retrieved from the device. The retrieved cells are further processed for molecular profiling. The platform may be run in multiple different modes. In the training/validation mode, the collected images of a sample may be fed to the AIAIA, configured to use unsupervised learning to group cells into sub-clusters. In some examples, the sub-clusters be morphologically distinct sub-clusters. In some examples, the cells may be grouped using deep learning embeddings (e.g., embeddings 952a, 952b, 964). Using AIAIA, a user may clean up the sub-clusters by removing cells that are incorrectly clustered and annotates each cluster based on a predefined annotation schema. The annotated cell images are then integrated into the Cell Morphology Atlas (CMA), a growing database of expert-annotated images of single cells. The CMA is broken down into training and validation sets and is used to train and evaluate CNN models aimed at identifying cell types, cell states, morphometric features, and/or the like. Under the analysis mode (view (d) of FIG. 14), the collected images are fed into models that had been previously trained using the CMA, and a report is generated demonstrating the composition of the sample of interest. A UMAP visualization is used to depict the morphometric map of all the single cells within the sample. A set of prediction probabilities is also generated showing the classifier prediction of each individual cell within the sample belonging to every predefined cell class within the CMA. In the sorting mode (view (c) of FIG. 14), the collected images are passed to the CNN in real-time and a decision is made on the fly to assign each single cell to one of the predefined classes within the CMA. In some examples, the collected images are passed to the CNN in real-time and the decision is made on the fly to assign each single cell using deep learning embeddings (e.g., embeddings 952a, 952b, 964) within the CMA. The target cells are then sorted in real-time and are outputted for downstream molecular assessment.

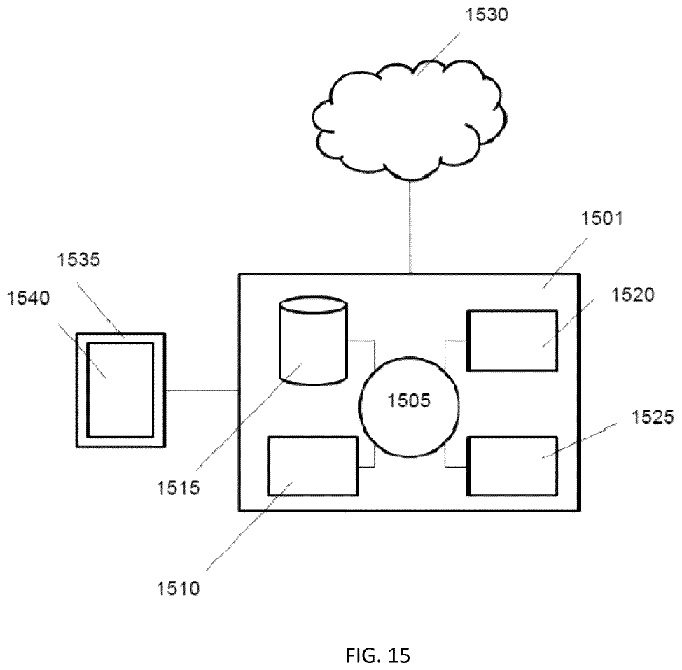

FIG. 15 shows a computer system that is programmed or otherwise configured to implement methods provided herein. For example, the present disclosure provides computer systems that are programmed to implement methods of the disclosure. FIG. 15 shows a computer system 1501 that is programmed or otherwise configured to capture and/or analyze one or more images of the cell. The computer system 1501 may regulate various examples of components of the cell sorting system of the present disclosure, such as, for example, the pump, the valve, and the imaging device. The computer system 1501 may be an electronic device of a user or a computer system that is remotely located with respect to the electronic device. The electronic device may be a mobile electronic device.

The computer system 1501 includes a central processing unit (CPU, also “processor” and “computer processor” herein) 1505, which may be a single core or multi core processor, or a plurality of processors for parallel processing. The computer system 1501 also includes memory or memory location 1510 (e.g., random-access memory, read-only memory, flash memory), electronic storage unit 1515 (e.g., hard disk), communication interface 1520 (e.g., network adapter) for communicating with one or more other systems, and peripheral devices 1525, such as cache, other memory, data storage and/or electronic display adapters. The memory 1510, storage unit 1515, interface 1520 and peripheral devices 1525 are in communication with the CPU 1505 through a communication bus (solid lines), such as a motherboard. The storage unit 1515 may be a data storage unit (or data repository) for storing data. The computer system 1501 may be operatively coupled to a computer network (“network”) 1530 with the aid of the communication interface 1520. The network 1530 may be the Internet, an internet and/or extranet, or an intranet and/or extranet that is in communication with the Internet. The network 1530 in some cases is a telecommunication and/or data network. The network 1530 may include one or more computer servers, which may enable distributed computing, such as cloud computing. The network 1530, in some cases with the aid of the computer system 1501, may implement a peer-to-peer network, which may enable devices coupled to the computer system 1501 to behave as a client or a server.

The CPU 1505 may execute a sequence of machine-readable instructions, which may be embodied in a program or software. The instructions may be stored in a memory location, such as the memory 1510. The instructions may be directed to the CPU 1505, which may subsequently program or otherwise configure the CPU 1505 to implement methods of the present disclosure. Examples of operations performed by the CPU 1505 may include fetch, decode, execute, and writeback.

The CPU 1505 may be part of a circuit, such as an integrated circuit. One or more other components of the system 1501 may be included in the circuit. In some cases, the circuit is an application specific integrated circuit (ASIC).

The storage unit 1515 may store files, such as drivers, libraries and saved programs. The storage unit 1515 may store user data, e.g., user preferences and user programs. The computer system 1501 in some cases may include one or more additional data storage units that are external to the computer system 1501, such as located on a remote server that is in communication with the computer system 1501 through an intranet or the Internet.

The computer system 1501 may communicate with one or more remote computer systems through the network 1530. For instance, the computer system 1501 may communicate with a remote computer system of a user. Examples of remote computer systems include personal computers (e.g., portable PC), slate or tablets, telephones, Smart phones (e.g., Apple® iPhone, Android-enabled device, Blackberry®), or personal digital assistants. The user may access the computer system 1501 using the network 1530.

Methods as described herein may be implemented by way of machine (e.g., computer processor) executable code stored on an electronic storage location of the computer system 1501, such as, for example, on the memory 1510 or electronic storage unit 1515. The machine executable or machine readable code may be provided in the form of software. During use, the code may be executed by the processor 1505. In some cases, the code may be retrieved from the storage unit 1515 and stored on the memory 1510 for ready access by the processor 1505. In some situations, the electronic storage unit 1515 may be precluded, and machine-executable instructions are stored on memory 1510.

The code may be pre-compiled and configured for use with a machine having a processor adapted to execute the code, or may be compiled during runtime. The code may be supplied in a programming language that may be selected to enable the code to execute in a pre-compiled or as-compiled fashion.

Examples of the systems and methods provided herein, such as the computer system 1501, may be embodied in programming. Various examples of the technology may be thought of as “products” or “articles of manufacture” typically in the form of machine (or processor) executable code and/or associated data that is carried on or embodied in a type of machine readable medium. Machine-executable code may be stored on an electronic storage unit, such as memory (e.g., read-only memory, random-access memory, flash memory) or a hard disk.

“Storage” type media may include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 1501 may include or be in communication with an electronic display 1535 that comprises a user interface (UI) 1540 for providing, for example, the one or more images of the cell that is transported through the channel of the cell sorting system. In some cases, the computer system 1501 may be configured to provide a live feedback of the images. Examples of Uis include, without limitation, a graphical user interface (GUI) and web-based user interface.

Methods and systems of the present disclosure may be implemented by way of one or more algorithms. An algorithm may be implemented by way of software upon execution by the central processing unit 1505. The algorithm may include, for example, the human foundation model.

It will be appreciated that the features and operations described herein may be used in any suitable combination with one another. An example method includes extracting, using a Deep Learning (DL) model (e.g., DL encoder 950), a set of machine learning (ML)-based features from a cell image (e.g., images 912 and/or augmented images thereof such as in batches 942a, 942b). As one example, any suitable component(s) may include a processor and a non-computer readable medium storing the machine learning model and related encoder, and instructions for causing the processor to perform operations. For example, the processor may be included with a cloud-based computing environment and/or within a microfluidics platform (e.g., platform 20, 310). Nonlimiting examples of machine learning encoders (such as deep learning encoders, for example, convolutional neural networks) are provided elsewhere herein. In some examples, cells in the one or more cell images are unstained. The one or more cell images are brightfield cell images in some examples, though it will be appreciated that other types of cell images readily may be provided to the machine learning encoder and computer vision encoder. The example method also may include generating, using the DL model and the set of ML-based features, a plurality of DL embeddings orthogonal to each other. The example method also may include extracting, using a computer vision model, a set of cell morphometric features from the cell image, the cell morphometric features being orthogonal to the plurality of DL embeddings. Some nonlimiting examples of cell morphometric features include cell position, cell shape, pixel intensity, texture, focus, or any combination thereof. These and other nonlimiting examples of cell morphological features are described in Table 2. The example method also may include generating a plurality of morphometric predictive embeddings using the set of ML-based features and the set of cell morphometric features. The morphometric predictive embeddings may be used in processing images of cells with different genetic edits than one another (e.g., CRISPR perturbations) in a manner such as described elsewhere herein.

Another example method includes extracting, using a Deep Learning (DL) model (e.g., DL encoder 950) and image data of a plurality of cells, a vector for a cell of the plurality of cells, the vector comprising a set of machine-learning (ML)-based features and a set of cell morphometric features extracted using a computer vision mode. As one example, any suitable component(s) may include a processor and a non-computer readable medium storing the machine learning model and related encoder, and instructions for causing the processor to perform operations. For example, the processor may be included with a cloud-based computing environment and/or within a microfluidics platform (e.g., system 100 or platform 310). Nonlimiting examples of machine learning encoders (such as deep learning encoders, for example, convolutional neural networks) are provided elsewhere herein. In some examples, cells in the one or more cell images are unstained. The one or more cell images are brightfield cell images in some examples, though it will be appreciated that other types of cell images readily may be provided to the machine learning encoder and computer vision encoder. The method also may include generating, using the DL model and using the set of ML-based features, a plurality of DL embeddings orthogonal to each other. The embeddings may be used in processing images of cells with different genetic edits than one another (e.g., CRISPR perturbations) in a manner such as described elsewhere herein.

### NON-LIMITING WORKING EXAMPLES

The following examples are intended to be purely illustrative, and not limiting of the present subject matter.

### Example 1—System for Cell Morphology Analysis

Table 3 in this Example lists parameters and specifications of an example system such as described with reference to FIG. 1 (e.g., The Deepcell® REM-I system by Deepcell, Inc., Menlo Park) for cell morphology analysis as well as image analysis generally, cell sorting, and other operations described herein. Table 4 lists the example components of one example system. “*” in Table 3 denotes the specification is dependent on sample characteristics and/or sorting configurations.

TABLE 3

| Category | Parameter | Specification |
|---|---|---|
| Facilities | Instrument dimensions | H: 29.5 in/75 cm |
| | | W: 35.5 in/90 cm |
| | | D: 29.5 in/75 cm |
| | Included ancillary | Computer tower |
| | equipment | Monitor |
| | | Keyboard |
| | | Mouse |
| | Electrical | 3 × 100-240 V surge- |
| | | protected outlets |
| | Network connection | 1 Gbps ethernet with >150 |
| | | Mbps upload bandwidth |
| | Clean dry air connection | 0.55-0.72 Mpa/80-105 psi |
| | Temperature operation | 59-86° F./15-30° C. |
| | ranges | |
| | Operating relative | 15-70, non-condensing |
| | humidity (%) | |
| Instrument | Input cell size (pm) | 6-25 |
| | Output cell viability (% of | >95* |
| | input viability) | |
| | Output collection | 6x positive outlet wells |
| | | 1x negative outlet tube |
| | | 1x waste bottle |
| Instrument | Positive outlet capacity | Up to 3,000 |
| | (cells/well) | |
| | Recommended run time | Live cells: Up to 180 |
| | (min) | |
| | | Fixed cells: Up to 600 |
| | Image resolution | 0.16 |
| | (pm/pixel) | |
| | Image size (pixels) | 256 × 256 |
| | Imaging throughput | Up to 1,000 |
| | (events/s) | |
| | Sorting throughput | Up to 30 |
| | (cells/s) | |

TABLE 4
Example Components of the REM-I system.

| Name | Description |
|---|---|
| REM-I Instrument | Microfluidic instrument |
| REM-I Imaging Kit | Reagents and consumables for imaging workflow |
| REM-I Sorting Kit | Reagents and consumables for imaging plus sorting |
| | workflow |
| Human Foundation | Artificial intelligence (AI) model for high- |
| Model (HFM) | dimensional single-cell morphology analysis and |
| | computer-vision model |
| Data Suite | Data suite for visualizing, analyzing, and storing |
| | data |

### Example 2—Processing Cells with Different Genetic Edits

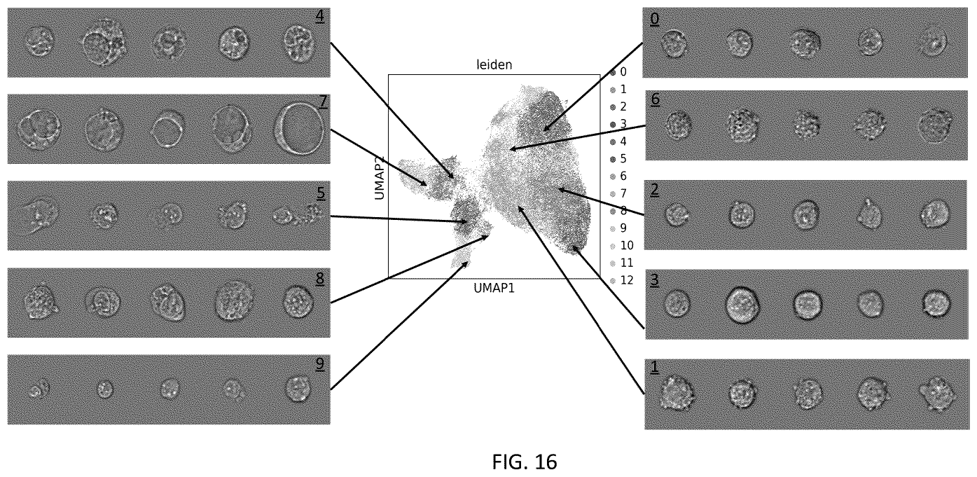

The REM-I platform was used to obtain images of cells with different genetic edits than one another. More specifically, a library of 11 single gene KOs was generated in three cell lines: HEK293 (Human Embryonic Kidney cells), Jurkat (T Lymphocyte), and K562 (Myelogenous Leukemia). Within each of these three cell lines, a negative control cell line was generated by exposing the respective HEK293, Jurkat, or K562 cells to Cas9 without a guide RNA for use in generating a genetic edit. The human foundation model 180, described with reference to FIG. 1, using 115 deep learning and morphometric features, was trained to extract features from the images. Individual genes for knockout using CRISPR-Cas9 were selected based on previously reported effects on cell morphology, such as vesicle formation, cell size, and nuclear size. The data suite 190, described with reference to FIG. 1, was trained using the multi-dimensional feature vectors from the cell lines and used to generate reduced-dimensionality plots which will be described with reference to FIGS. 16, 17, 18, 19, 20, and 21.

TABLE 5
Cells of FIGS. 1, 16, and 17

| Cell designation (Leiden | | Gene knockout (most |
|---|---|---|
| cluster) | Cell line | prevalent) |
| 0 | HEK293 | None (control) |
| 1 | HEK293 | Various (BUB1, FBX05, |
| | | STK11, WDR45, |
| | | YAP1) |
| 2 | HEK293 | Various (FBX05, INO80, |
| | | STK11, YAP1) |
| 3 | HEK293 | Various (STK11, TSC1, |
| | | WDR45) |
| 4 | HEK293 | VAC14 |
| 5 | HEK293 | COG4 |
| 6 | HEK293 | Various (STK11, YAP1, |
| | | WDR45) |
| 7 | HEK293 | FIG4 |
| 8 | HEK293 | COG4 |
| 9 | HEK293 | YAP1 |
| 10 | HEK293 | None (Control) |
| 11 | HEK293 | Various (INO80, STK11) |
| 12 | HEK293 | Various (BUB1, STK11, |
| | | WDR45) |

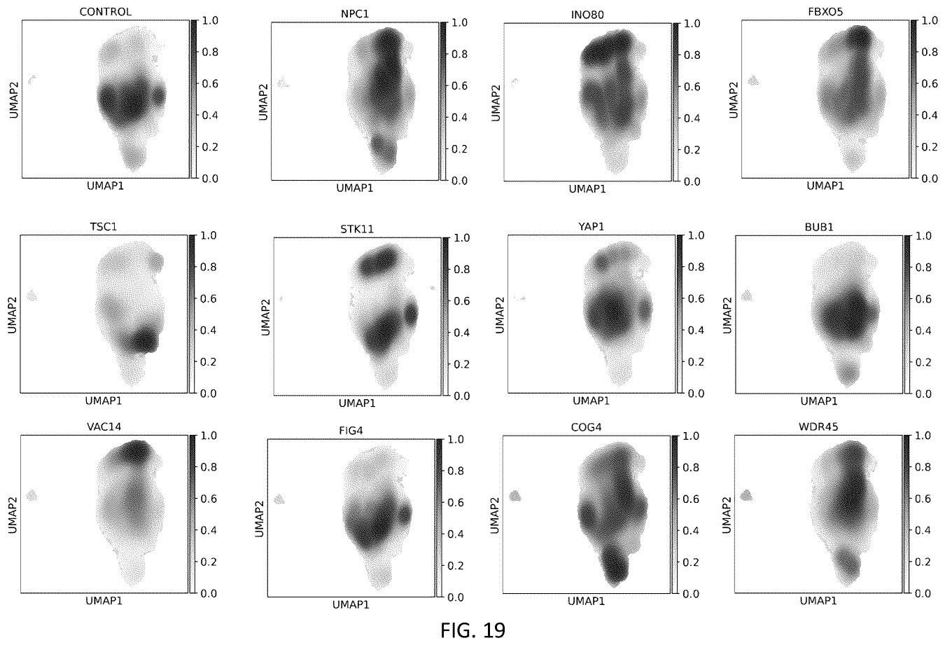

TABLE 6
Cells of FIGS. 18 and 19

| Cell designation (Leiden | | |
|---|---|---|
| cluster) | Cell line | Gene knockout |
| 0 | K562 | None (control) |
| 1 | K562 | Various (COG4, FBX05, |
| | | INO80, NCP1, STK11, |
| | | VAC14, WDR45) |
| 2 | K562 | Various (None- Control, |
| | | COG4, FBX05, FIG4, |
| | | STK11, YAP1) |
| 3 | K562 | Various (COG4, FBX05, |
| | | IN080, NPC1, WDR45) |
| 4 | K562 | Various (BUB1, COG4, |
| | | NPC1, WDR45) |
| 5 | K562 | Various (None- Control, |
| | | COG4, INO80, YAP1) |
| 6 | K562 | Various (None- Control, |
| | | BUB1, COG4, FIG4, |
| | | STK11, YAP1) |
| 7 | K562 | Various (COG4, INO80, |
| | | STK11) |
| 8 | K562 | TSC1 |
| 9 | K562 | Various (COG4, INO80, |
| | | NPC1) |
| 10 | K562 | Various (COG4, WDR45) |

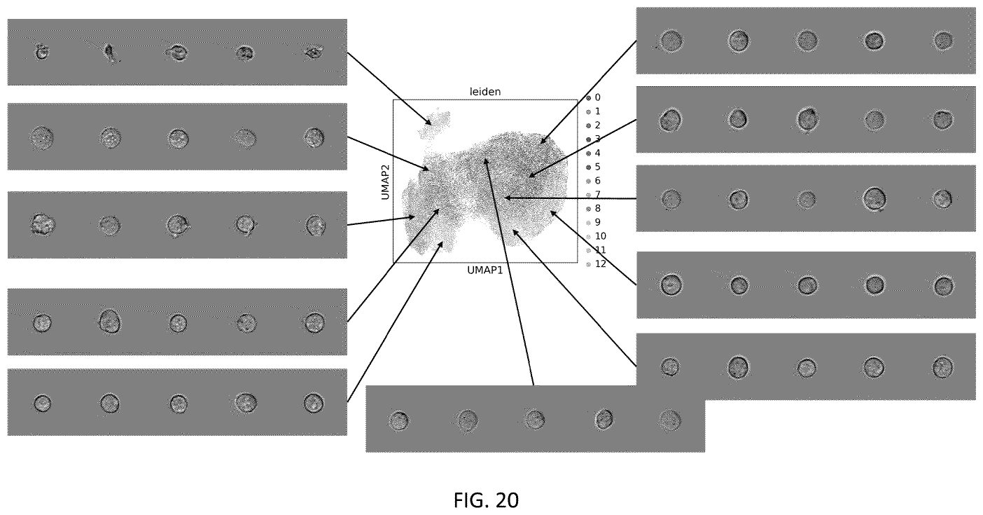

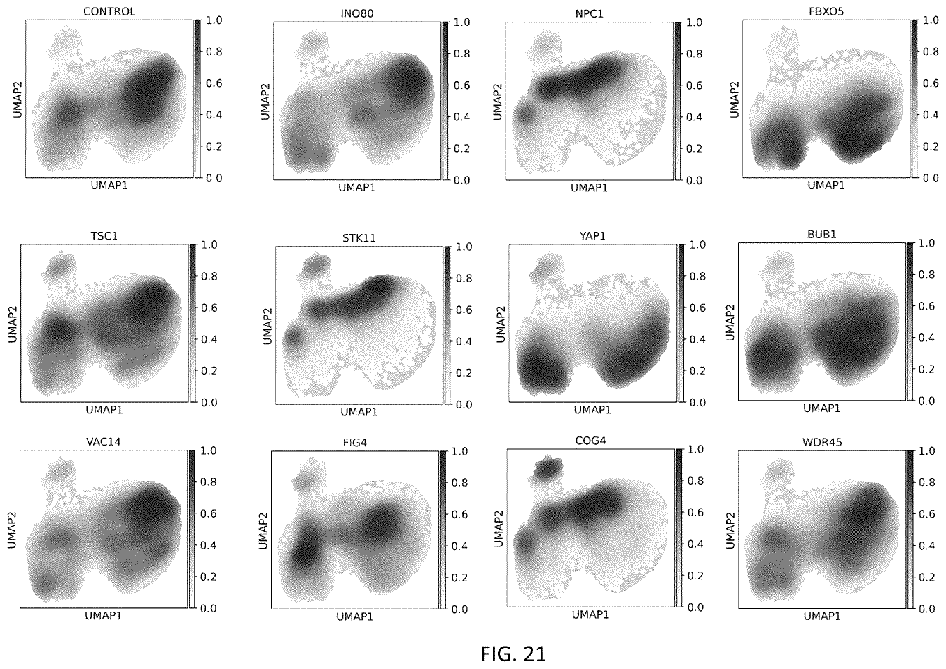

TABLE 7
Cells of FIGS. 20 and 21

| Cell designation (Leiden | | Gene knockout (most |
|---|---|---|
| cluster) | Cell line | prevalent) |
| 0 | Jurkat | Various (COG4, NPC1, |
| | | STK11) |
| 1 | Jurkat | Various (BUB1, FBX05, |
| | | INO80, TSC1, VAC14, |
| | | WDR45, YAP1) |
| 2 | Jurkat | Various (BUB1, FIG4, |
| | | TSC1, VAC14, |
| | | WDR45) |
| 3 | Jurkat | Various (None- Control, |
| | | FIG4, INO80, TSC1, |
| | | VAC14, WDR45) |
| 4 | Jurkat | Various (BUB1, FBXO5, |
| | | FIG4, INO80, TSC1, |
| | | VAC14, WDR45) |
| 5 | Jurkat | Various (None- Control, |
| | | INO80, TSC1, VAC14, |
| | | WDR45) |
| 6 | Jurkat | Various (BUB1, YAP1) |
| 7 | Jurkat | Various (BUB1, FBXO5, |
| | | YAP1) |
| 8 | Jurkat | Various (COG4, NPC1, |
| | | STK11) |
| 9 | Jurkat | COG4 |
| 10 | Jurkat | Various (BUB1, FBXO5, |
| | | INO80, YAP1) |
| 11 | Jurkat | Various (COG4, NPC1, |
| | | STK11) |
| 12 | Jurkat | Various (COG4, NPC1, |
| | | STK11) |

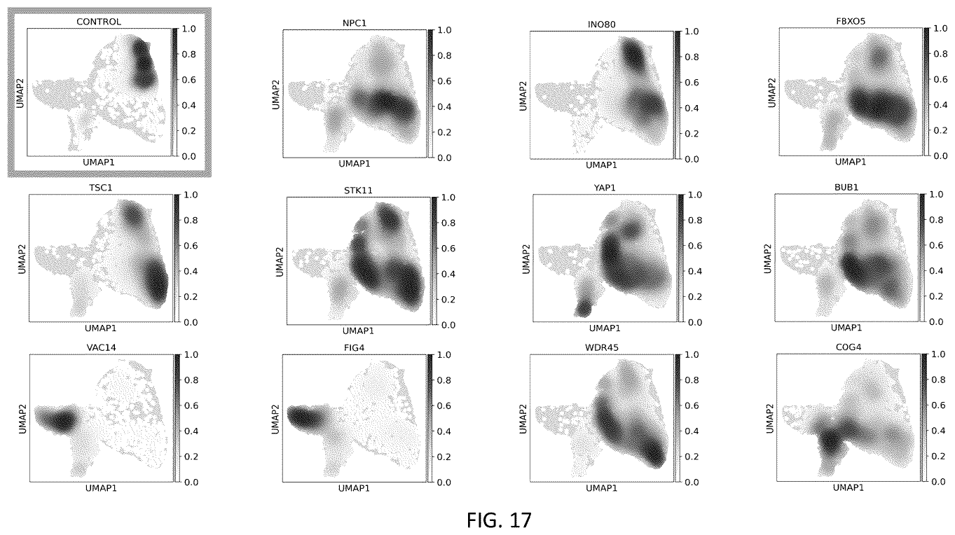

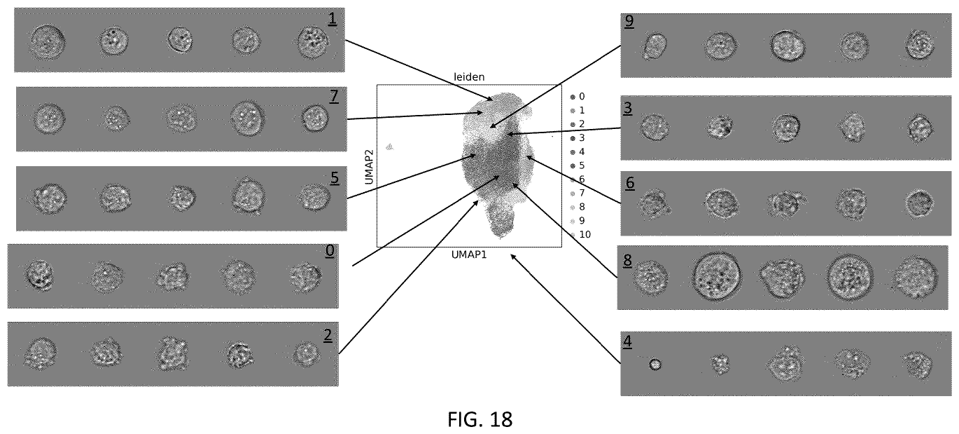

Single gene KOs were found to result in significant morphological changes relative to control cells. For example, FIG. 16 illustrates example reduced-dimensionality plots of features extracted from images of cells with different genetic edits than one another, and example images of cells having certain features. As illustrated in FIG. 16, the data suite generated clusters 0, 1, 2, . . . 12 corresponding to phenotypes resulting from different ones of the genetic edits to HEK293 cells described in Table 5. The data suite 190 was used to display the images of cells within each of the clusters. From the images shown in FIG. 16, it may be understood that the different genetic edits generated different cellular phenotypes that the data suite 160 identified and clustered. FIG. 17 illustrates example reduced-dimensionality plots of features extracted from images of cells with different genetic edits than one another, namely the same cells as described with reference to FIG. 16 and Table 5. From FIGS. 16 and 17 and Table 5, it may be understood that there are overlaps between the cells with genetic edits contained within each Leiden cluster. For example, Leiden cluster 3 contains STK11, TSC1, WDR45 KO cells. Similarly, it may be understood that there are overlaps between cellular phenotypes and Leiden clusters. For example, in FIG. 16 the control, is in majority located in Leiden cluster 0 and in Leiden cluster 10. From the density plot in FIG. 17, it may be understood that cell size increases were identified in Leiden clusters 6, 1, 2, 3 and 8. It also may be understood that KO cells with increased nuclei size are also in the cell size cluster, showing that increased nuclei size also may be correlated with an increase in cell size. It also may be observed that the WDR45 KO cells and in a smaller measure the COG4 KO cells also experienced cell size increases. On the other hand, cells with gene KOs affecting vesicle formation are located in Leiden cluster 7, 4, 5, and 9, which may be understood from the density plots of FIG. 17 and Leiden clusters in FIG. 16. From these results, it may be understood that the present systems and methods may be used to associate clusters (e.g., Leiden clusters) with a range of morphotype or morphological changes (brought by KO). It was a surprising result to observe that genes not known to be involved in cell size regulation (e.g. WDR45 and COG4) were able to induce significant changes in cell size. This result indicates that morphology can provide powerful phenotype and gene function information that otherwise would be difficult to glean with conventional methods.

In some cases, genetic edits may induce pronounced, subtle, or no morphological changes in cells. This information in of itself is useful for developing cell type specific atlases of the link between morphology and gene function. One main utility of such atlases, for instance, would be to infer gene function for uncharacterized genes; in other words, gene KOs with known function may be used as references on a projection, while a gene with unknown function is projected as well, and a user may see how the cells are grouped together with the reference cells.

FIG. 18 illustrates example reduced-dimensionality plots of features extracted from images of cells with different genetic edits than one another, and example images of cells having certain features. As illustrated in FIG. 18, the data suite generated clusters 0, 1, 2, . . . 12 corresponding to phenotypes resulting from different ones of the genetic edits to K562 cells described in Table 6. The data suite 190 was used to display the images of cells within each of the clusters. From the images shown in FIG. 18, it may be understood that the different genetic edits generated different cellular phenotypes that the data suite 160 identified and clustered. FIG. 19 illustrates example reduced-dimensionality plots of features extracted from images of cells with different genetic edits than one another, namely the same cells as described with reference to FIG. 18 and Table 6. Control is located in cluster 0, and a lesser extent in cluster 2. The interesting part in K562 is the subtle change in morphology (compared to HEK). For example, the FIG. 4 and VAC14 KO cells of HEK display a relatively clear morphotype, while the K562 KO cells (see, e.g., images for clusters 6 and 2 in FIG. 18) display more subtle morphological changes. For example, morphological changes to VAC14 are relatively subtle in K562 compared to HEK, although a clear morphological shift may be observed in cluster 1. From these data, it may be understood that the present systems and methods may be used to detect subtle (as well as less subtle) changes in morphology, thus extending the power of detecting cell morphology induced by gene KO and mutations.

FIG. 20 illustrates example reduced-dimensionality plots of features extracted from images of cells with different genetic edits than one another, and example images of cells having certain features. As illustrated in FIG. 20, the data suite generated clusters 0, 1, 2, . . . 12 corresponding to phenotypes resulting from different ones of the genetic edits to Jurkat cells described in Table 7. The data suite 190 was used to display the images of cells within each of the clusters. From the images shown in FIG. 20, it may be understood that the different genetic edits generated different cellular phenotypes that the data suite 160 identified and clustered. FIG. 21 illustrates example reduced-dimensionality plots of features extracted from images of cells with different genetic edits than one another, namely the same cells as described with reference to FIG. 20 and Table 7. From these data, it may be understood that the present systems and methods may be used to detect subtle (as well as less subtle) changes in morphology, thus extending the power of detecting cell morphology induced by gene KO and mutations.

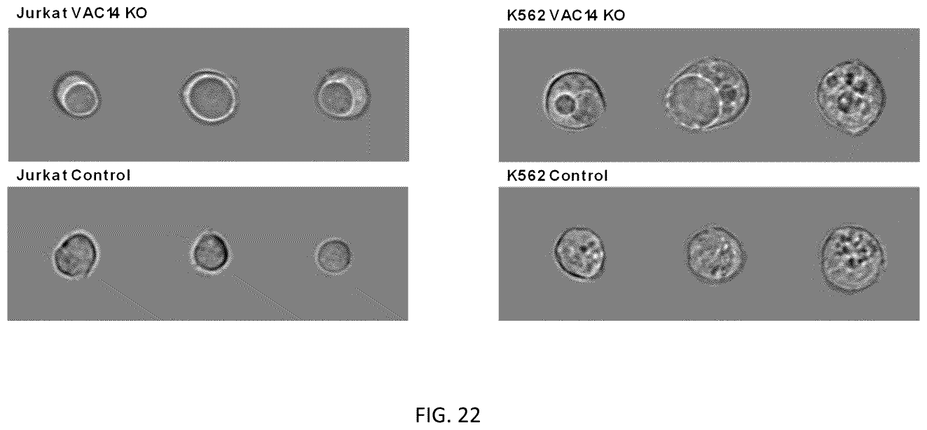

From these data, it may be understood that the REM-I platform captured high-dimensional functional resolution of single gene KOs, including distinct responses from cell line to cell line, indicating the same gene perturbation translates to different phenotypic effects in accordance to cell context. Consistent phenotypic patterns in KOs from similar gene expression pathways were found within the same cell lines, suggesting that morphology as a functional readout is robust and orthogonal. Several rare morphological phenotypes (e.g., about 1-10% of the population) were also observed, which may give further insight into the relative importance of a single gene in each cell context. For example, FIG. 22 illustrates example images of cells with different genetic edits than one another. More specifically, FIG. 22 illustrates images of the Jurkat VAC14 KO, the Jurkat control, the K562 VAC14KO, and the K562 control. The majority of the Jurkat VAC14 KO cells were observed to be morphologically similar to the Jurkat control cells. However, a subpopulation of about 4-6% of the Jurkat VAC14 KO cells—of which the subpopulation of the Jurkat VAC14 KO cells shown in FIG. 22 are representative—were morphologically distinct from the Jurkat control cells, and indeed resembled the HEK293 VAC14 KO cells and the K562 VAC14 KO cells. Additionally, the majority of the K562 VAC14 KO cells were observed to be morphologically similar to the K562 control cells. However, a subpopulation of about 1-2% of the K562 VAC14 KO cells—of which the subpopulation of the K562 VAC14 KO cells shown in FIG. 22 are representative—were morphologically distinct from the K562 control cells, and indeed resembled the HEK293 VAC14 KO cells.

From these results, it may be understood that deep learning-driven morphology analysis may be used to analyze a wide range of phenotypes induced by genetic perturbations, such as CRISPR KOs.

### ADDITIONAL COMMENTS

While certain examples of the present subject matter have been shown and described herein, it will be obvious to those skilled in the art that such examples are purely illustrative. It is not intended that the invention be limited by the specific examples provided within the specification. While nonlimiting examples of the invention has been described with reference to the aforementioned specification, the descriptions and illustrations of the examples herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the examples described herein may be employed in practicing the invention. It is therefore contemplated that the claims shall also cover any such alternatives, modifications, variations, or equivalents.

Additional aspects and advantages of the present disclosure will become readily apparent to those skilled in this art from the following detailed description, wherein only illustrative examples of the present disclosure are shown and described. As will be realized, the present disclosure is capable of other and different examples, and its several details are capable of modifications in various obvious respects, all without departing from the disclosure. Accordingly, the drawings and description are to be regarded as illustrative in nature, and not as restrictive.

1.-32. (canceled)

33. A method of processing, the method comprising:
using a machine learning encoder to extract respective sets of machine learning (ML)-based features from respective images of viable, unstained cells, wherein cells of a first subset of the cells have a first genetic edit, and wherein cells of a second subset of the cells lack the first genetic edit;
using a computer vision encoder to extract respective sets of cell morphometric features from the respective images;
using the respective sets of ML-based features and the respective sets of cell morphometric features to generate respective multi-dimensional feature vectors that represent respective cell phenotypes; and
using the respective multi-dimensional feature vectors to correlate, to the first genetic edit, a phenotypic difference between the cells of the first subset and the cells of the second subset.

34. The method of claim 33, wherein the first genetic edit comprises a first gene knockout.

35. The method of claim 34, wherein the first gene knockout is generated using Clustered Regularly Interspaced Short Palindromic Repeats (CRISPR) and a CRISPR-associated (Cas) protein.

36. The method of claim 33, wherein the cells comprise a plurality of additional subsets having different genetic edits than one another.

37. The method of claim 36, wherein the genetic edits are genome-wide.

38. The method of claim 36, wherein the cells comprise at least 100 additional subsets having different genetic edits than one another.

39. The method of claim 33, wherein the cells of the second subset of the cells have a second genetic edit that is different from the first genetic edit.

40. The method of claim 33, wherein the cells of the second subset of the cells comprise a control.

41. The method of claim 33, further comprising:
inputting the cells of the first subset of the cells to an inlet of a fluidic channel;
flowing the cells of the first subset of the cells from the inlet through the fluidic channel; and
generating the respective images of the cells of the first subset of the cells within the fluidic channel.

42. The method of claim 41, wherein the cells of the first subset of cells are pooled with the second subset of cells, such that inputting the cells of the first subset of the cells to the inlet of the fluidic channel further comprises inputting the cells of the second subset of the cells to the inlet of the fluidic channel.

43. The method of claim 41, wherein the cells of the second subset of the cells are input to the inlet of the fluidic channel separately from the cells of the first subset of the cells.

44. The method of claim 41, further comprising collecting the cells of the first subset of the cells at an outlet of the fluidic channel.

45. The method of claim 44, wherein the outlet comprises a first and second reservoirs, the method further comprising physically sorting the cells into the first reservoir or into the second reservoir using the phenotypic difference between the cells of the first subset and the cells of the second subset.

46. The method of claim 45, further comprising performing a molecular characterization of the cells sorted into the first reservoir.

47. The method of claim 46, wherein the molecular characterization is selected from the group consisting of: single-cell RNA sequencing (scRNA-seq), single cell gene expression, single cell Assay for Transposase Accessible Chromatin (ATAC), combined single cell ATAC and gene expression, combined single cell gene expression and cell surface markers or intracellular proteins, single cell examination, bulk gene expression, bulk ATAC, bulk cell examination, or immunofluorescence.

48. The method of claim 33, wherein the correlating comprises informatically linking the first genetic edit to a feature in the multi-dimensional feature vectors that is present in the cells of the first subset and is not present in the cells of the second subset.

49. The method of claim 33, wherein the machine learning encoder uses a convolutional neural network or a vision transformer.

50. The method of claim 33, wherein the ML-based features are orthogonal to one another, to the cell morphometric features, or both.

51. The method of claim 33, wherein the cell morphometric features are selected from the group consisting of position features, cell shape features, pixel intensity features, texture features, and focus features.

52. The method of claim 33, further comprising reducing dimensionalities of the multi-dimensional feature vectors to generate lower-dimensional vectors, wherein the lower-dimensional vectors are used to correlate, to the first genetic edit, the phenotypic difference between the cells of the first subset and the cells of the second subset.

53. The method of claim 52, wherein the correlating comprises informatically linking the first genetic edit to a feature cluster in a space defined by the lower-dimensional vectors that is present in the cells of the first subset and is not present in the cells of the second subset.
